(19) United States
(12) Patent Application Publication
(10) Pub. No.: US 2016/0097134 A1
(43) Pub. Date: Apr. 7, 2016

(54) Conversion of Pharmaceuticals and Chemicals into different Compounds by Polarity Reversal Electrolysis

(71) Applicant: Abdul R.M. Azad, Northborough, MA (US)

(72) Inventor: Abdul R.M. Azad, Northborough, MA (US)

(21) Appl. No.: 14/966,751

(22) Filed: Dec. 11, 2015

Related U.S. Application Data
(63) Continuation-in-part of application No. 14/964,247, filed on Dec. 9, 2015.
Application No. 14/964,247 is a continuation of application No. PCT/US14/41531, filed on Jun. 9, 2014.
(60) Provisional application No. 61/833,095, filed on Jun. 10, 2013.
(60) Provisional application No. 62/090,871, filed on Dec. 11, 2014.

(51) Int. Cl. C25B 3/10; A61K 45/06; C25B 9/04; A61K 31/231; C25B 11/12; C25B 11/04

(52) U.S. Cl. CPC C25B 3/105; C25B 11/12; C25B 11/0426; C25B 9/04; A61K 31/231; A61K 45/06

(57) ABSTRACT

A polarity-reversal electrolysis process. The process uses a reactor that has at least one pair of spaced electrodes, and a controlled polarity-reversing power supply that is constructed and arranged to provide polarity-reversed power to the electrodes. An electrically-conductive liquid reaction medium that includes precursor reactants is provided to the reactor. The electrodes are at least partially immersed in the reaction medium. The power supply is operated such that the polarity of the electrodes of the pair of electrodes reverses at a frequency rate, so as to produce reactive intermediates and products.

### CROSS-REFERENCE TO RELATED APPLICATIONS

This application is a continuation in part of and claims priority to application Ser. No. 14/964,247 filed on Dec. 9, 2015, which is a continuation of and claimed priority of PCT/US14/41531 filed on Jun. 9, 2014, which itself claimed priority of Provisional Patent Application Ser. No. 61/833,095, filed on Jun. 10, 2013. This application also claims priority of Provisional Patent Application Ser. No. 62/090,871, filed on Dec. 11, 2014. The disclosures of all of these prior applications are incorporated by reference herein.

### FIELD

This invention relates to the conversion of chemicals by polarity reversal electrolysis.

### BACKGROUND

Pharmaceuticals and specialty chemicals require specific physical, chemical, functional properties that are inherent in the chemical structure. While these chemical entities can be prepared using known chemical methods, many cannot be easily prepared or cannot be prepared economically. There is therefore a need for new and modified processes for producing pharmaceuticals and chemicals economically on an industrial scale.

Electrolysis is known in the art as a method for performing chemical reactions on a laboratory scale and selected processes have reached industrial scales. Electrolysis of carboxylic and fatty acids and decarboxylation have been reported by Kolbe to form alkanes, called the Kolbe dimer. Laboratory experiments where a normal Kolbe reaction is prevented by drastically changed reaction conditions have been reported in the prior art, beginning with Moest et. al. (German patent 138442, issued 1903) who created alcohols, aldehydes and ketones from fatty acids using electrolysis.

Kronenthal et al focused on aliphatic ethers, and on methoxy-undecane in particular (U.S. Pat. No. 2,760,926, issued in 1956), but achieved yields of 40% or less while consuming large amounts of electricity (by at least a factor ten judging from the voltage applied (90+Volts).

More recently, however, in US20060773279P and WO2007027669 the original Kolbe-reaction was quoted as a means, among numerous other techniques, to create useful hydrocarbons utilizing fatty acids of renewable origin. However due to the nature of the Kolbe-reaction, the chain length would almost double in the process, creating a mix of C30-C34 hydrocarbons that would need extensive conventional refining to yield useable, liquid transportation fuels. This may be contrasted with a one-step specialized Hofer-Moest process, where an alkene is produced, however at low current densities and at low productivity rates. However, even under these conditions considerable Kolbe dimer is formed. Furthermore, alkenes with terminal unsaturation are readily subject to oxidation, and decreases the oxidation stability. Additional recent publications are PCT/US2008/010707, PCT Pub No. WO2009/035689, U.S. Pat. No. 8,444,846 B2, issued May 21, 2013 and JOSHI; CHANDRASHEKHAR H.; Homer; Michael Glenn; United States Patent Application, 20120197050, A1, Publication, Aug. 2, 2012.

There have been references related to the use of alternating current for the electrolysis of aqueous solutions using sine waves compared to direct current. For example U.S. Pat. No. 2,385,410 issued Sep. 25, 1945 to John Albert Gardner, describes a method of producing organic disulphides which consists in treating an aqueous solution of an alkali metal salt or alkaline earth metal salt of a mercapto thiazole or a dithiocarbamic acid by electrolysis with alternating current whereby the hydroxide of the alkali or alkaline earth metal is liberated and the disulphide is formed by the union of the residues from two molecules. However, generally, those skilled in the art are aware that in direct current electrolysis, electrons flow in the same direction all the time, whereas in alternating current, the electrons flow one way (typically 1/60 of a sec in 60 Hz sine wave alternating current) and then they flow the other way. To get any electrolysis that is not immediately undone, direct current is required.

It would be possible to manufacture said pharmaceuticals and chemicals by means of a regular, crossed Kolbe-electrolysis, e.g. using oleic acid and acetic acid as feedstock. This procedure would yield a C18-hydrocarbon and would maintain the configuration of the double bond of the fatty acid. However, it is believed that such a technique would be far less economical due to the consumption of acetic acid, the costly use of platinum anodes, and the low-value byproducts. (i.e. ethane and a doubly unsaturated C34 hydrocarbon in this case) generally unavoidable in a crossed or hetero Kolbe reaction.

It has been reported that many companies are cooperating with producers of animal fat and/or vegetable oils to create hydrocarbons from triglycerides, making straight C16/C18 alkanes and propane (from the glycerol contained in fats/oils). However, this process uses a catalyst and totally hydrogenates feedstock at high pressures and temperatures. It consumes large amounts of hydrogen, requires catalysts and destroys all special configurations of the fatty acid originated double bonds. The process described in this invention can preserve such double bonds and can utilize the hydrogen generated by the electrolysis. The need for an external source of hydrogen is avoided.

### SUMMARY

The invention relates to a process for the conversion and production of decarboxylated derivatives from carboxylic acids by replacing the carboxylic group with hydrogen, an alkyl group, an alkene group, an alkoxy group, an aryloxy group, aryl group or hydroxyl group. The invention also has utility in the conversion of organic cations, radicals and anions such as carboxylic acids, fatty acids, alcohols, phenols, drugs, pharmaceuticals, controlled release agents, specialty chemicals, and surfactants useful as pharmaceuticals and chemicals. The invention relates to novel compositions that can be obtained by the novel process. The inventions also relates to an apparatus for carrying out the process and producing the novel compositions. More specifically, the invention relates to a process for producing modified pharmaceuticals and chemicals and coupled products to be used as a chemicals, pharmaceuticals, from organic anions and carboxylic acids. It also concerns production of an ether, an alkane, an alkene, an alkyl-aryl hydrocarbon, an alcohol and ester compounds as derivatives, and the use of the derivatives as pharmaceuticals, chemicals, surfactants and with modified physical and chemical properties. The invention is particularly concerned with the field of pharmaceuticals with modified solubility, immunogenicity, toxicity, stability and effectiveness

More specifically, the invention relates to a process for producing modified pharmaceuticals and chemicals and coupled products to be used as a chemicals, pharmaceuticals, from organic anions and carboxylic acids. It also concerns production of an ether, an alkane, an alkene, an alkyl-aryl hydrocarbon, an alcohol and ester compounds as derivatives, and the use of the derivatives as pharmaceuticals, chemicals, surfactants and with modified physical, chemical and pharmacological properties. The invention is particularly concerned with the field of pharmaceuticals with modified solubility, immunogenicity, toxicity, stability and pharmacological effectiveness.

The invention provides a process for decarboxylation of a carboxylic acid and anions such as an aliphatic, cyclic, heterocyclic or aromatic carboxylic acids to produce the corresponding decarboxylated and anion-free derivatives, such as hydrocarbons comprising alkanes, alkenes, alkyl-aryl hydrocarbons, hydrocarbon ethers or hydrocarbon esters in organic solvents, and the use of the derivatives as pharmaceuticals and chemicals. In addition, specific other reagents can be coupled to the pharmaceutical or chemical to further modify the radical, radical anion or radical cation produced in the process. More generally, the invention can be used to produce organic radical cations, neutral radicals, cations and anions as reactive intermediates for further reaction with added solvents, functional groups and other additives. More specifically, the invention is directed for producing pharmaceutical intermediates, pharmaceuticals and chemicals that cannot be economically made using other methods. In addition, the invention also discloses decarboxylated and deionized compositions that can be used by the chemical, pharmaceutical and fuel industry and apparatus for carrying out the invention.

The invention also includes compositions that can be produced by the inventive process by decarboxylation including hydrocarbon compositions, alkanes, alkenes, alkyl-aryl hydrocarbons, hydrocarbon ethers, hydrocarbon alcohols and hydrocarbon esters. Product compositions can be selected by selecting the initial reagents, solvents and additives.

The invention also discloses an apparatus for carrying out the inventive process, in a batch process, semi-continuous process and a continuous process.

In particular the invention provides a process for producing pharmaceutical compositions and chemicals which comprises the step of performing polarity reversing electrolysis on a solvent solution of an anion such as a carboxylic acid or salt thereof or carboxylic acid ester or other derivative or precursor thereof to decarboxylate said carboxylic acid or derivative thereof, and produce a decarboxylated derivative product.

In particular, the initial objective of the invention provides a process for producing a decarboxylated derivative, such as a pharmaceutical or chemical, which comprises the step of performing polarity reversing electrolysis with an anode and a cathode on a solvent of an anion, a carboxylic acid or salt thereof or carboxylic acid ester or other derivative or precursor thereof to form a reactive radical intermediate or to decarboxylate said carboxylic acid or derivative, and produce the corresponding decarboxylated product or adduct with the radical intermediate. In addition, the process conditions can be adjusted to produce alkyl-aryl hydrocarbons, alkenes, ethers, alcohols and esters in one step. Another inventive step is the composition of the solvent and carboxylic acid concentration such that the products of the electrolysis phase separates from the initial homogeneous reaction mixture and greatly simplifies the separation and purification of the products. This avoids or reduces substantially the need for separation using multiple separation steps. In addition, the reaction medium containing solvents and salts can be reused to decarboxylate additional carboxylic acids and modify ionic compounds without the need for additional reagents and solvents. Furthermore, the polarity reversal process overcomes the mass transfer limitations of direct current electrolysis and reduces electrode fouling by the products. Catalysts such as platinum, palladium nickel can be coated or impregnated onto the electrodes to further enhance the electrolysis.

By a precursor of a carboxylic acid (or of a salt or other derivative thereof) or ionic compound is referred to a compound that will produce such a material under appropriate reaction conditions, in particular under the conditions under which electrolysis is to be carried out. An example of a suitable precursor is an ester that hydrolyses in situ. Other examples are carboxylic acid derivatives that allow for electrical conductivity and electrolysis, such as carboxylic acid salts, carboxylic acids, phenols and tertiary amines, both free and immobilized on solid supports that produce carboxylate anions and anions. In addition, the tertiary amines can produce anions from the alcohol solvents to produce ethers during decarboxylation.

The invention also provides a product composition or compositions produced directly or indirectly by this process that can be used by the chemical, the pharmaceutical, agricultural and the specialty chemical industry.

The decarboxylated product compositions that is produced may be used to prepare other chemicals, pharmaceutical intermediates, fuels or fuel additives.

The process of the invention may further comprise the steps of purifying and separating the products from the reactive intermediates generated and decarboxylated product compositions from the reaction solvent. (Additionally, the process of the invention may further comprise the step of adding the decarboxylated product alkane to a fuel to produce renewable fuel or as fuel additives to the fuel.)

A further objective is to overcome the great limitation of mass transfer and ion-transport that limits the efficiency and productivity of direct current (DC) electrolysis. In DC electrolysis unidirectional ion transport is needed to complete the circuit. In AC electrolysis, this mass transfer limitation is avoided. Furthermore, the desired product is produced only at the anode or at the cathode, and undesired product is not used or wasted. Another objective of the invention is to improve the efficiency and productivity of electrolysis that is not possible with DC electrolysis, due to the fouling of the electrodes and decreased current density of the electrolysis. The above limitations can be overcome by disclosed invention as demonstrated in the examples. Furthermore, the use of different polarity reversing functions such as square wave function overcomes the limitations of sine wave AC electrolysis.

### BRIEF DESCRIPTION OF THE FIGURES

FIG. 1 is a description of the apparatus and process flow diagram illustrating a manufacturing process of the invention that may be used in a batch as well as continuous process.

FIG. 2 is a description of the apparatus and process flow diagram illustrating a manufacturing process of the invention that may be used in a batch as well as continuous process where the reaction mixture is subjected to sonication or mechanical stirring to improve mass transfer.

FIG. 3 is a description of the apparatus and process flow diagram illustrating a manufacturing process of the invention using multiple electrodes that may be used in a batch as well as continuous process to increase productivity.

FIG. 4 is a description of the apparatus and process flow diagram illustrating a manufacturing process of the invention using electrodes that may be used in a flow through continuous process.

FIGS. 5-8 are data.

FIG. 9 is a description of the apparatus with nanotube electrodes and catalysts with process flow diagram illustrating a manufacturing process of the invention using electrodes that may be used in a flow through continuous process.

### DETAILED DESCRIPTION

The decarboxylation of the anion or carboxylic acid group of the carboxylic or fatty acid by reverse polarity electrolysis generates a reactive intermediate such as a decarboxylated radical intermediate at both the anode and cathode during the anodic cycle of each electrode, and produces a hydrogen radical at the cathode during the cathodic cycle of each electrode. In addition, carbocations (carbonium ions) can be produced at each electrode during the anodic cycle depending on the applied voltage and the ionization potential of the molecule. Without speculating on the mechanism of the invention, in order to understand the invention, it is possible, under some electrolysis reaction conditions, for the Kolbe reaction to occur, whereby the radical dimerises by reaction with another alkyl radical of the same type to produce a Kolbe dimer. If the frequency of the polarity reversal is low, there is sufficient time for the radical to react with another radical, dimerise and for the formation of the normal Kolbe dimer product. However, when the frequency of the polarity reversal is high, when the anode changes polarity, and the alkyl radical in the vicinity of the anode is now in the proximity of the polarity changed cathode. The cathode reduces hydrogen ions to hydrogen radicals that react with the alkyl radicals in the vicinity produced in the prior cycle to produce the alkane. This allows for the use of the hydrogen that is normally produced in the prior art Kolbe reaction to react directly with the alkyl radical and produce the alkane. This avoids the need to use a low molecular weight acid such as acetic, propionic or formic acid to produce a Kolbe low molecular weight dimer.

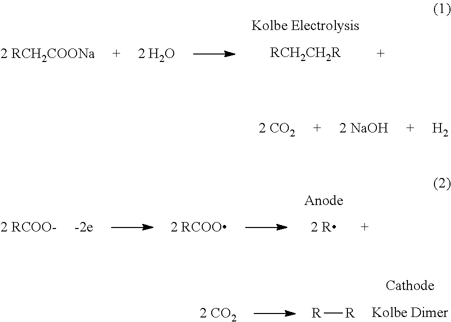

Represents Free Radical

It is known in the art that to form the normal Kolbe dimer with two alkyl radicals, high current densities and high carboxylate concentrations are needed presumably to produce sufficiently high concentrations of radicals available on the electrode surface or in the vicinity for reaction. It is also known that at low current densities and higher electrode potentials, the alkyl radicals abstract hydrogen from the neighboring carbon atom and form an alkene, the Hofer-Moest reaction.

2RCH₂—CH₂—COO— −2e→2RCH₂—CH₂.−2e→2RCH₂—CH₂+ (3)

### Carbocation

2RCH₂—CH₂+→2RCH═CH₂+H—H Hofer-Moest (4)

2RCH₂—CH₂+H—H→2RCH₂CH₃Hofer-Moest (5)

2RCH₂—CH₂+2CH₃O→2RCH₂CH₂—OCH₃ Hofer-Moest (6)

The conditions used for electrolysis in the Hofer-Moest process can be selected to generate a low concentration of free radicals, which minimizes the occurrence of free radical dimerization and thereby reduces the Kolbe reaction, but is not prevented. However, low current densities result in low reaction and production rates, and the hydrogen is released and lost with its energy. Furthermore, hydrogen is still generated and released at the cathode and requires schemes to capture and utilize the hydrogen.

### Electrolysis

2RCH₂CH₂COONa+2H₂O→2RCH═CH₂+2CO₂+2NaOH+H₂ (7)

### The Anode Cycle

2RCH₂CH₂COO— −2e→2RCH₂CH₂.+2CO₂ (8)

### The Cathode Cycle

2H+ +2e→2H.

2H₂O+2e→2OH⁻+H₂

2Na+ +2e→2Na+2H₂O→2OH⁻+H₂ (9)

### The Reaction on and in the Vicinity of the Anode and Cathode

2RCH₂CH₂.+2H.→2RCH₂CH₂—H→2RCH₂CH₃ (10)

### Non-Kolbe Alkane

Overall Reaction with Polarity Reversal Electrolysis.

2RCH₂CH₂COOH+2NaOH→2RCH₂CH₂COONa+2H₂O (11)

2RCH₂CH₂COONa+2H₂O→2RCH₂CH₃+2CO₂+2NaOH (12)

### Anode-Cathode

2RCH₂CH₂COO— −2e→2RCH₂CH₂.+2CO₂ (13)

2RCH₂CH₂.−2e→2RCH₂CH₂+→2RCH═CH₂+H—H (14)

### Carbocation on Further Oxidation at Anode

2RCH₂CH₂+ +2CH₃O—→2RCH₂CH₂OCH₃ (15)

2RCH₂CH₂.+2e→2RCH₂CH₂— (16)

### Carbanion on Further Reduction at Cathode at Cathode Cycle

2RCH₂CH₂—+2H+→2RCH₂CH₂—H (17)

In general, besides the carboxylate anion and hydrogen ion, metal ion or amine cation, any anion or cation that that can interact with the anode and cathode can be used. Therefore, it is preferred that the anions and cations present in the electrolyte solution are restricted only to those that are desired to prevent the formation of unwanted side products.

The invention can be described generally as given below.

### Anode/Anode Cycle: A− is the Anion

A− −e→A.−e→A+ Carbocation

A.+A.→AA Kolbe Dimer (18)

A.+B.→AB (19)

A+ +Nu−→ANu (20)

### Nu− is a Nucleophile

### Cathode/Cathode Cycle: B+ is the Cation

B+ +e→B.+e→B− Carbanion (21)

B.+A.→AB (22)

B− +E+→BE (23)

### E+ is an Electrophile

Besides the radical reactions, A. and B., the carbocation and carbanion can then react with any nucleophile (Nu−) or electrophile (E+) present in its vicinity to produce the corresponding products.

In the process of the invention, the free radicals such as the alkyl free radicals generated by decarboxylation of the fatty acid react with a nearby hydrogen radical produced during the cathodic cycle to produce an alkane. If a reactive solvent molecule is present, such as an alcohol, the alkoxy free radical or an anion can react with the alkyl radical to produce an ether. The alkyl radical may eliminate a hydrogen atom to form an alkene and an alkane. In principle, the alkyl radicals could also be further oxidized (i.e. loose another electron) and become carbocations, which may undergo structural changes before either reacting with the hydrogen radical or hydrogen ion to form an alkane, with the solvent to form an ether or eliminating a hydrogen atom to form an alkene before the polarity reversal. A mixture of ethers, a mixture of alkanes and alkenes and esters can sometimes be obtained from the process of the invention, and the formation of the Kolbe dimer is minimized. The number of carbon atoms in the alkanes and alkenes is one less than the number of carbon atoms in the carboxylic acid (the carboxyl group of the fatty acid splits off as CO₂).

A number of factors may influence the nature and concentration of the radicals, cations and anions that are produced during the polarity reversing electrolysis step. These factors include the size and shape of the electrodes, the material from which the electrodes are made, the surface characteristics of the electrodes, the distance separating the electrodes in solution, the electrolyte and solvents that are used, the concentration of the reactants such as carboxylic acid, the properties of the carboxylic acid salt, type of current, direct or with polarity reversal, the function and shape of the applied voltage and the polarity switch, the rise and fall times of the polarity reversal frequency, the symmetry of the polarity reversal function, the electrode potential voltage and the current density. In addition, the formation of organic radical cations, neutral radicals, cations and anions is specific to each molecule, and dependent on the ionization energy and the bond dissociation energy among other factors. Thus, the electrolysis step may be performed in a number of different ways in order to obtain the desired product or products and to produce the alkane, the alkene, ether or ester as described in the invention and to minimize the Kolbe dimer. I The conditions also influence the amounts of alkane, ether and alkene that are produced. If an alkyl ether is not desired, a non-alcoholic solvent can be used. If an alkene is not desired, current density, voltage, frequency of the polarity switch, polarity switch function, and voltage function can be changed to obtain predominantly the desired products.

The general reactions given in equations (18) to (23) is further illustrated in Table I for the different molecules that may form reactive intermediates for further reaction to form products. For example, acetic acid, CH₃COOH acetic acid radical CH₃COO. acetate anion CH₃COO⁻ from Table I can undergo decarboxylation similar to the experimental examples given for oleic acid.

TABLE I
Organic Radical Cations, Neutral Radicals, Cations, and Anions
Reactive Intermediates that may be generated and undergo reactions under
polarity reversal Electrolysis Conditions that may undergo further reaction.

Hydrocarbons
C1
methane, CH₄ methylene singlet, CH₂ methylene triplet, CH₂** methyl radical,
CH₃• methyl cation, CH₃⁺ methyl anion, CH₃⁻
C2
ethane, CH₃CH₃ ethane radical cation, CH₃CH₃⁺• ethyl radical, CH₃CH₂• ethyl cation,
CH₃CH₂⁺ ethyl anion, CH₃CH₂⁻
ethylene, CH₂═CH₂ ethylene radical cation, CH₂═CH₂⁺• vinyl radical, CH₂═CH• vinyl
cation, CH₂═CH⁺
acetylene, HCCH deH-acetylene radical, HCC• acetylene anion, HCC⁻
C3
propane, CH₃CH₂CH₃ propane radical cation, CH₃CH₂CH₃⁺• propyl radical,
CH₃CH₂CH₂• propyl cation, CH₃CH₂CH₂⁺
cyclopropane, CH₂(CH₂)CH₂ cyclopropane radical, CH₂(CH₂)CH₂•
isopropyl cation, (CH₃)₂CH⁺ isopropyl radical, (CH₃)₂CH•
propene, CH₂═CHCH₃ propene radical cation, CH₂═CHCH₃⁺• 1-deH-1-propene radical,
CH₃CH═CH• 2-deH-propene cation, CH₂═C(+)CH₃ 1-deH-1-propene cation,
CH₃CH═CH⁺
allyl cation, CH₂•CHCH₂⁺ allyl radical, CH₂•CHCH₂• allyl anion, CH₂═CHCH₂⁻
C4
butane, CH₃CH₂CH₂CH₃ butane radical cation, CH₃CH₂CH₂CH₃⁺• 1-deH-butane radical,
CH₃CH₂CH₂CH₂•
2-deH-butane radical, CH₃CH₂CH(•)CH₃ 1-deH-butane cation, CH₃CH₂CH₂CH₂⁺ 2-deH-
butane cation, CH₃CH₂CH(+)CH₃
2-methylpropane, (CH₃)₃CH 2-methylpropane radical cation, (CH₃)₃CH⁺•
isobutyl cation, (CH₃)₂CHCH₂⁺ isobutyl radical, (CH₃)₂CHCH₂•
2-methylpropene, (CH₃)₂C═CH₂ 2-methylpropene radical cation, (CH₃)₂C═CH₂⁺• 2-deH-1-
methylcyclopropane cation, CH₂(CH+)CHCH₃ 3-deH-butene cation,
CH₂═CHC(+)HCH₃ 2-deH-methylpropene cation, CH₂═CH(CH₃)CH₂⁺ 1-deH-1-
methylcyclopropane cation, CH₂(CH₂)C(+)CH₃
t-butyl radical, (CH₃)₃C• t-butyl cation, (CH₃)₃C⁺
C5
2-methylbutane, (CH₃)₂CHCH₂CH₃ 2-methylbutane radical cation,
(CH₃)₂CHCH₂CH₃⁺• isopentyl radical, (CH₃)₂C(•)CH₂CH₃
isopentyl cation, (CH₃)₂C(+)CH₂CH₃ 1-pentene, H₂C═CHCH₂CH₂CH₃ 1-pentene radical
cation, H₂C═CHCH₂CH₂CH₃⁺•
C6
2-methylpentane radical cation, CH₃CH(CH₃)CH₂CH₂CH₃⁺•
2,2-dimethylbutane, (CH₃)₃CCH₂CH₃ 2,2-dimethylbutane radical cation, (CH₃)₃CCH₂CH₃⁺•
Aromatics
Benzene, C₆H₆, Benzene Radical Cation, C₆H₆⁺• Benzene Radical Anion, C₆H₆⁻•,
Toluene, C₇H₈, Toluene Radical Cation, C₇H₈⁺• Toluene Radical Anion, C₇H₈⁻•,
Phenyl Radical, C₆H₅• Tolyl Radical, C₆H₇⁻•,
Oxygen: Alcohols, Ethers, Aldehydes, Ketones, and Acids
C1
methanol, CH₃OH methanol radical cation, CH₃OH⁺• methoxy radical,
CH₃O• methylperoxyl radical, CH₃OO•
methoxy cation, CH₃O⁺ methanol onium cation, CH₃OH₂⁺ methoxy anion, CH₃O⁻ 1-deH-
methanol radical, CH₂•O
formaldehyde, CH₂O formyloxonium cation, CH₂OH⁺ deH-formaldehyde cation, HCO⁺
deH-formaldehyde anion, HCO⁻ deH-formaldehyde radical, HCO•
formic acid, HCOOH 1-deH-formic acid radical, HOOC• formate anion, HCOO⁻
C2
ethanol, CH₃CH₂OH O-deH-ethanol radical, CH₃CH₂O• ethoxy anion, CH₃CH₂O⁻
methyl acylium cation, CH₃CO⁺
ethyleneoxide, C₂H₄O deH-ethyleneoxide cation, C₂H₃O⁺
dimethylether radical, CH₃OCH₂• dimethylether cation, CH₃OCH₂⁺
vinyl alcohol, CH₂═CHOH vinyl alcohol radical cation, CH₂═CHOH⁺• vinyloxy radical,
CH₂═CHO• vinyloxy anion, CH₂═CHO⁻
1-deH-acetaldehyde cation, CH₃CO⁺ 1-deH-acetaldehyde radical, CH₃CO•
2-deH-acetaldehyde cation, O═CHCH₂⁺ 2-deH-acetaldehyde radical, O═CHCH₂•
acetic acid, CH₃COOH acetic acid radical, CH₃COO• acetate anion, CH₃COO⁻
deH-acetic acid radical, HOOCCH₂• deH-acetic acid cation, HOOCCH₂⁺ O-deH-acetic
acid cation, CH₃COO⁺
C3
propanol, CH₃CH₂CH₂OH propanol radical cation, CH₃CH₂CH₂OH⁺• 1-deH-propanol
radical, CH₃CH₂CH•OH
1-deH-propanol cation, CH₃CH₂CH(+)OH 2-deH-propanol radical, CH₃CH•CH₂OH 2-
deH-propanol cation, CH₃CH(+)CH₂OH
3-deH-propanol cation, CH₂(+)CH₂CH₂OH isopropanol, (CH₃)₂CHOH isopropanol radical
cation, (CH₃)₂CHOH⁺•
methylethylether, CH₃OCH₂CH₃ methylethylether radical cation, CH₃OCH₂CH₃⁺•
deH-methylethylether radical, CH₃CH₂OCH₂• deH-methylethylether cation,
CH₃CH₂OCH₂⁺
propanal radical cation, CH₃CH₂CO⁺• 1-deH-propanal cation, CH₃CH₂CO⁺ 1-deH-
propanal radical, CH₃CH₂CO•
acetone, CH₃C═OCH₃ deH-propanone cation, CH₃C═OCH₂⁺ deH-propanone radical,
CH₃C═OCH₂•
propenol radical cation, H₂C═COHCH₃⁺• allyl alcohol radical, HOCH═CHCH₂•
methylacetate, CH₃COOCH₃ methylacetate radical cation, CH₃COOCH₃⁺•
methyl-deH-acetate radical, CH₃OOCCH₂• methyl-deH-acetate cation, CH₃OOCCH₂⁺
propanoic acid, CH₃CH₂COOH propanoic acid radical, CH₃CH₂COO•
2-deH-propanoic acid radical, CH₃CH•COOH 2-deH-propanoic acid cation,
CH₃CH(+)COOH
C4-C5 Alcohols, Ethers
C4
2-methylpropanol, (CH₃)₂CHCH₂OH 2-methylpropanol radical cation,
(CH₃)₂CHCH₂OH⁺•
2-deH-2-methylpropanol radical, (CH₃)₂C•CH₂OH 2-methylpropoxy radical,
(CH₃)₂CHCH₂O•
t-butanol, (CH₃)₃COH t-butanol radical cation, (CH₃)₃COH⁺•
t-butyloxy cation, (CH₃)₃CO⁺ 2-deH-isopropylmethylether cation, (CH₃)₂C(+)OCH₃ t-
butyloxy radical, (CH₃)₃CO•
diethylether, CH₃CH₂OCH₂CH₃ diethylether radical cation, CH₃CH₂OCH₂CH₃⁺•
ethylvinylether, CH₃CH₂OCH═CH₂ ethylvinylether radical cation, CH₃CH₂OCH═CH₂⁺•
C5
methyl-t-butylether, (CH₃)₃COCH₃ methyl-t-butylether radical cation, (CH₃)₃COCH₃⁺•
C4-C5 Aldehydes, Ketones, Carboxylic Acids
C4
butyraldehyde, CH₃CH₂CH₂CHO butyraldehyde radical cation, CH₃CH₂CH₂CHO⁺•
1-deH-butyraldehyde radical, CH₃CH₂CH₂CO• 1-deH-butyraldehyde cation,
CH₃CH₂CH₂CO⁺
2-butanone, CH₃CH₂COCH₃ 2-butanone radical cation, CH₃CH₂COCH₃⁺•
methylpropionate, CH₃CH₂COOCH₃ methylpropionate radical cation, CH₃CH₂COOCH₃⁺•
C5
2-pentanone, CH₃CH₂CH₂COCH₃ 2-pentanone radical cation, CH₃CH₂CH₂COCH₃⁺•
pentenol radical cation, CH₃COH═CH₂CH₂CH₂⁺•
Nitrogen
methylamine, CH₃NH₂ N-deH-methylamine radical, CH₃NH• methaniminonium ion,
CH₂NH₂⁺ methylamide anion, CH₃NH⁻ methylammonium radical, CH₃NH₃•
2-aminobutane, CH₃CH(NH₂)CH₂CH₃ 2-aminopropyl radical, CH₃CH(NH₂)CH₂• imine,
CH₂═NH
N-deH-ethanimine radical, CH₃CH═N• acetonitrile, CH₃CN 1-deH-acetonitrile radical,
NCCH₂• 1-deH-acetonitrile anion, NCCH₂⁻
propylimmonium cation, CH₃CH₂CH═NH₂⁺ 2-aminopropyl cation, CH₃CH(NH₂)CH₂⁺
dimethylamine, CH₃NHCH₃ dimethylamine radical cation, CH₃NHCH₃⁺• dimethylamine
radical, CH₃N•CH₃
N-methylmethaniminonium cation, CH₃NH═CH₂⁺
formamide, HCONH₂ formamidate anion, HCONH⁻ N-methylacetamide,
CH₃C═ONHCH₃
N-methylacetamide radical cation, CH₃C═ONHCH₃⁺• N-methylacetamide radical,
CH₃C═ON•CH₃
N-methylacetamide cation, CH₃C═ON(+)CH₃
N N-dimethylacetamide, CH₃C═ON(CH₃)₂ N,N-dimethylacetamide radical cation,
CH₃C═ON(CH₃)₂⁺•
Halogens
difluorocarbene singlet, CF₂ methylfluoride cation, CH₂F⁺ methylchloride cation,
CH₂Cl⁺ methylchloride anion, CH₂Cl⁻
ethylfluoride, CH₃CH₂F 1,1-difluoroethane, CH₃CHF₂ ethylfluoride radical cation,
CH₃CH₂F⁺•
fluoroethylene, CH₂CHF 1-deH-1-fluoroethylene anion, CH₂CF⁻
chloroethane, CH₃CH₂Cl 1-deH-1-chloroethane radical, CH₃CH(•)Cl chloroethylene,
CH₂CHCl
bromooethane, CH₃CH₂Br 1-deH-1-bromoethane radical, CH₃CH(•)Br
anti-dichloroethane radical cation, anti-C₂H₄Cl₂⁺•
allylchloride radical, ClCH═CHCH₂• 1-chloropropane radical, ClCH₂CH•CH₃
chloroacetic acid, ClCH₂COOH chloroacetate anion, ClCH₂COO⁻
Allylic
allyl cation, CH₂═CHCH₂⁺ allyl radical, CH₂═CHCH₂• allyl⁻ allylalcohol
radical allylchloride radical 1-chloropropane radical
Small Radicals and Ions
OH radical HOO radical HCO₃ radical CO₃⁻ radical
Stable Neutral Molecules
Hydrides: H₂O HCN HNCO HOCN HNCS HSCN HF HCl
Footnote:
The above list of organic reactive intermediates (Table I) arc representative of the cations, radicals and anions that can be formed by the disclosure and that can react.
Table data is referenced from literature.

In this embodiment, the radical, the carbocation can act as an electrophile and subsequently involved in the electrophilic substitution reaction. In such a reaction, the electrophile substitutes one of the substituents on an aromatic group, for example, hydrogen, instead of the hydrogen generated by the electro-reversal, as shown below as a non-limiting example with benzene.

RCOO— −e→+R.+CO₂ (24)

R.−e→R+ (25)

R₁⁺+C₆H₆→C₆H₅R₁+H⁺ (26)

R₁.+C₆H₆→C₆H₅R₁+H. (27)

The H⁺ or the H. may then be consumed, further reacted, etc., in the reactor in the vicinity of the electrodes or in the bulk solution. In the embodiment shown above, benzene is shown as the aromatic solvent or additive, instead of water or n-hexane given in the examples. Those skilled in the art will appreciate that other aromatics such as toluene or non-aromatic organic solvents or additives may also be used, that will react with the radical or the carbocation.

### Pharmaceuticals and Modified Pharmaceuticals

There is a need for new drug candidates and drug discovery for targeted therapies and personalized medicine. The disclosed invention expands the availability of potential drug candidates for targeted and personalized medicines as well. Currently, for practical and economic reasons, most labs sequence only tumor DNA to identify clinically actionable mutations. This practice may lead to inappropriate use of genomic data to guide cancer therapies and adversely impact patients and lead to greater downstream healthcare costs, and an overall improvement in targeted therapies is needed. Analyses of cancer genomes have revealed mechanisms underlying tumorigenesis and new avenues for therapeutic intervention. After discovering new durable targets in disease, new drugs are needed for effective therapies by matching patients to clinical trials to provide investigational therapies that has the greatest chance of success.

The carboxylic acid functional group can be an important component of a pharmacophore, however, the presence of this moiety can also be responsible for significant drawbacks, including metabolic instability, toxicity, as well as limited passive diffusion across biological membranes. To avoid some of these shortcomings while retaining the desired attributes of the carboxylic moiety, medicinal chemists often investigate the use of carboxylic acid (bio)isosteres. The same type of strategy can also be effective for a variety of purposes, for example to increase the selectivity of a biologically active compound, to increase its solubility, or to create new chemical entities useful as new pharmaceuticals. Generally, screening of a panel of new chemical entity candidates is required. In this context the discovery and development of novel pharmaceuticals using the invention could complement the current pharmaceuticals. In this context, the discovery and development of novel carboxylic acid surrogates that could complement the existing palette of isosteres remains an important area of research. Some non-limiting examples of the invention are given below.

5-oxo-1,2,4-oxadiazole systems containing —COOH groups can be modified to replace the —COOH specifically with a —OH, OR, OCH₂CH₂—O, NHR, CH2, R or SR group to impart desired properties.

The carboxylic acid functional group adds to the hydrophilicity of the drug as well as to its polarity and this may impede the bioavailability. Most carboxylic acids have a pKa value of about 3.5 to 4.5 and thus these compounds are ionized (deprotonated) under physiological conditions. These properties of carboxylic acids have been recognized for many years and a number of practical solutions have been identified with the most common being that of ester modification. The present invention gives an alternate route of carboxylic acid drug modification without the limitation of the hydrolysis of the ester under physiological conditions. The same invention can be used for the modification of drugs containing alcohol groups and phenolic gro ups.

(Drug Structure)-COOH+HOCH₂CH₂OCH₂CH₂OH→
(Drug Structure).+—OCH₂CH₂OCH₂CH₂OH+CO₂
(Drug Structure)-OCH₂CH₂OCH₂CH₂OH
D-OCH₂CH₂OCH₂CH₂OH
D-Additive, selected from Table 1, as organic radical cation, neutral radical, cation, anion or other desired radical cation, neutral radical, cation or anion.
D is the Therapeutic Drug moiety, and OCH₂CH₂OCH₂CH₂OH is the Additive.
In the case of a Protein, Peptide or other bio-pharmaceutical, D represents the Protein.

Non-limiting specific examples of drugs that can be modified are angiotensin-converting enzyme inhibitors, given below. Captopril, Lisonopril, Enalaprilat and Trandrapril are selected as specific examples.

Captopril: D-SH+HOOC—CH₂—CH₂—OH→D-S—CH₂—CH₂—OH (New Drug)
Lisinopril: D-NH₂+HOOC—CH₂—CH₂—OH→D-NH—CH₂—CH₂—OH (New Drug)
Enalaprilat: D-C₆H₅+HOOC—CH₂—CH₂—OH→D-C₆H₄—CH₂—CH₂—OH (New Drug)
Trandapril: D-COOH+HOOC—CH₂—CH₂—OH→D-CH₂—CH₂—OH (New Drug)

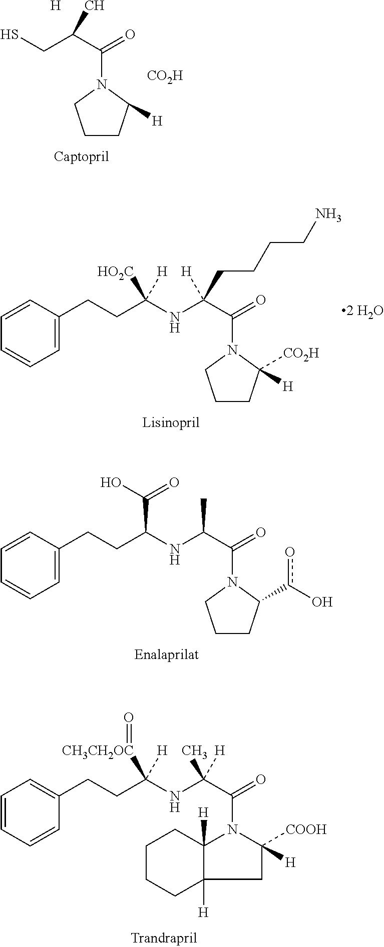

Another non-limiting specific examples of drugs that can be modified are antifungals, such as Polyene, Imidazoles, Triazoles, Thiazoles, Allylamines, Echinocandins and other antifungals. Antifungals work buy exploiting differences between mamamlian and fungal cells to kill the fingal organism with fewer adverse effects to the host. Fungal and human cells are similar at the biological level. This makes it more difficult to discoverdrugs that target fungi without affecting human cells. As a consequence, many antifungal drugs cause side-effects. Some of these side-effects can be life-threatening if the drugs are not used properly. The disclosed invention allows for the rapid modification of potential new drug candiates for optimum selection, or for the modification of existing drugs to form derivatives and to reduce the side effects.

Given below are specific examples of antifungals containing —OH and —RNH groups that can be modified using the invention.

(Anti-fungal)-OH+HOOC—CH₂—CH₂—OH→(Anti-fungal)-O—CH₂—CH₂—OH (New Drug)
(Anti-fungal)-RNH+HOOC—CH₂—CH₂—OH→(Anti-fungal)-RN—CH₂—CH₂—OH (New Drug)

### Bio-Pharmaceuticals and Modified Bio-Pharmaceuticals

Interferon and other peptides and proteins can be modified using the technology similar to the pharmaceuticals. The bio-pharmaceutical can be dissolved in a polar aprotic solvents such as dimethyl sulfoxide, dimethyl formamide and N-methyl pyrrolidone and subject to polarity reversal electrolysis. Additives may be selected from Table 1, as organic radical cation, neutral radical, cation, anion or other desired radical cation, neutral radical, cation or anion. A non limiting example and disclosure is given below as a representatiobe example.

A pharmaceutical or desired biopharmaceutical is dissolved in a polar aprotic solvent such as dimethyl sulfoxide, dimethyl formamide or N-methyl pyrrolidone. The desired additive group such as ethylene glycol, methanol or other desired additive is dissolved in the aprotic solvent and treated with an alkali metal such as sodium or potassium to form the alkoxide. The two solutions are them mixed and subject to polarity reversal electrolysis, similar to that described in the examples 1 to 8. In the case of pharmaceuticals and bio-pharmaceuticals, the oleic acid is replaced by the pharmaceutical or bio-pharmaceutical to be modified, and the solvent is selected from a polar aprotic solvent such as dimethyl sulfoxide, dimethyl formamide or N-methyl pyrrolidone. The additive is selected from the desired additives described on Tablel or any other desired additive. In the case of an alcohol, such an methanol, ethanol, ethylene glycol, polyethylene glycol, the sodium alkoxide can be used or prepared in-situ. If a mercapto group is desired, sodium methanethiolate or sodium ethanethiolate may be used.

### Anode/Anode Cycle: A− is the Anion

A.+B.→AB

(Drug Structure)-COOH+NaOCH₂CH₂OCH₂CH₂ONa→
(Drug Structure)-COON+—OCH₂CH₂OCH₂CH₂ONa→
(Drug Structure).+.OCH₂CH₂OCH₂CH₂ONa+CO₂→

### Neutral Radical

(Drug Structure)-OCH₂CH₂OCH₂CH₂ONa+CO₂→

A− −e→A.−e→A+ Carbocation

A+ +Nu−→ANu

(Drug Structure).+—OCH₂CH₂OCH₂CH₂ONa+CO₂→
(Drug Structure)+ +—OCH₂CH₂OCH₂CH₂ONa+CO₂→

### Organic Radical Cation Anion

### Cathode/Cathode Cycle: B+ is the Cation

B+ +e→B.+e→B− Carbanion (21)

B.+A.→AB (22)

B− +E+→BE (23)

### E+ is an Electrophile

(Drug Structure)-COOH+HOCH₂CH₂OCH₂CH₂OH→

### (Drug Structure).+H.+CO₂→

### (Drug Structure)-H+CO₂→

### (Drug Structure)-+H+ +CO₂→

### (Drug Structure)-H

(Drug Structure)-OCH₂CH₂OCH₂CH₂ONa+CO₂→
(Drug Structure)-OCH₂CH₂OCH₂CH₂ONa+CO₂→
(Drug Structure)-OCH₂CH₂OCH₂CH₂ONa
D-OCH₂CH₂OCH₂CH₂OH
(Drug Structure)-COOH+NaSCH₂CH₃→
(Drug Structure).+—SCH₂CH₃+CO₂
(Drug Structure)+ +—SCH₂CH₃+CO₂
(Drug Structure)-SCH₂CH₃
D-SCH₂CH₃

While in the above examples, OH and SH groups are used as anionic groups, any anionic group such as phenol, phosphate sulfate, sulfonate that can undergo reaction at the anode or cathode can be used in the invention.

A scheme for linking and modifying a biologically active compound or other chemical entity can be linked using the above invention by using a desired functional group on the active compound. Depending on the reactive functional group (Z1 or Z2) of the biologically active compound, a corresponding functional or additive group (Y1 or Y2) can be selected from Table I to provide linkages to modify to modify the biologically active compound or to link to a polymer for controlled release of the biologically active compound.

TABLE IB
A scheme for linking and modifying a biologically active compound

| Functional Group on the | Additive Group On | Resulting Linkage |
|---|---|---|
| Biologically Active | the link | to Biologically Active |
| Compound (Z₁ or Z₂) | (Y₁ or Y₂) | Compound |
| D-COOH => D• | HO—R₁=>*•O—R₁ | D-O—R₁ Ether |
| D-COOH => D• | CH2—(O)—CH2 | D-CH2—CH2—OH Alkyl-Alcohol |
| | Ethylene Oxide | |
| D-COOH => D• | CHR₁—(O)—CHR₂ | D-CHR₁—CH—(OH)R₂ Alkyl-Alcohol |
| | Epoxide | |
| D-COOH => D• | HS—R₁=>•S—R₁ | D-S—R₁ Thioether |
| D-COOH => D• | H₂N—CH₃=> •HN—CH₃ | D-NHCH₃ Amine |
| D-COOH => D• | HN—R₁R₂=> •N—R₁R₂ | D-N—R₁R₂ Amine |
| D-COOH => D• | CH₃CONHCH₃=> CH₃CON•CH₃ | D-N(CH₃)—COCH₃ |
| | N-methyl Acetamide Radical | Amide |
| D-COOH => D• | HO—R₁=> •O—R₁ | D-O—R₁ Ether |
| D-COOH => D• | HO—=> •OH | D-OH Alcohol |
| D-COOH => D• | ClCH₂COO— =>DOCOCH₂Cl | Ester |
| D-COOH => D• =>D+ | +ClCH₂COO— =>DOCOCH₂Cl | Ester |
| D-COOH => D• =>D+ | —OCH₃ =>DOCH₃ | Ether |
| D-COOH => D• + | CH₃C═ON(CH₃)₂⁺• | —OCH₃ =>DOCH₃ Ether |
| D-COOH => D• | P—CH₂COO— =>DOCOCH₂P | Ester |

P—Polymer for Polymer - Drug Conjugates

| | | |
|---|---|---|
| D-COOH => D• | +P—CH₂O• =>DOCH₂P | Ether |
| D-OH => DO• | +P—CH₂CO• =>DOCOCH₂P | Ester |
| D-OH => DO• | +P—CH₂• =>DOCH₂P | Ether |
| | (P—CH₂COOH => P—CH₂•) | |
| D-COOH => D• | +•OCH₂CH₂OH=> DO—CH₂CH₂OH | Ether |

Note:
D- represents the drug structure of a biologically active compound containing the functional group —COOH or —OH that can undergo further modification. Biologically active compound is a pharmaceutical drug that is a small molecule or a biological or a new molecular entity still under active discovery or investigation.

In the Table IB above, the biologically active compound was represented by Z₁ or Z₂ containing functional groups —COOH and —OH attached to the rest of the structural unit of the drug, D, other functional groups attached to the drug D, such as sulfhydral, —SH, primary amine, NH₂, secondary amine, —NHR₁, tertiary amine, —NR₁R₂, Ether, —O, halogens, F, Cl, Br, I, sulfates and sulfonates, —S═O, alkyl groups, —R, aromatic groups, Ar, heterocyclic groups, ester groups, —COOR, aldehyde groups, —CHO, and ketone, —COR₁R and other groups can be modified using the invention to modify current and to produce novel drugs to obtain desirable properties. The invention is applicable to the drug classes given below, whether a chemical entity or a biological.

### Drug Classes

A drug may be classified by the chemical type of the active ingredient or by the way it is used to treat a particular condition. Each drug can be classified into one or more drug classes.

TABLE IC
Drug Classes by indication that can be modified using Organic Radical
Cations, Neutral Radicals, Cations, and Anions using the current
invention.
(Reactive Intermediates that may be generated and undergo reactions
under polarity reversal Electrolysis Conditions that undergo further
reaction.)

```
allergenics
anti-infectives, amebicides, aminoglycosides, anthelmintics,
antifungals, azole antifungals, echinocandins, miscellaneous
antifungals, polyenes
    antimalarial agents, antimalarial combinations, antimalarial
  quinolines, miscellaneous antimalarials
    antituberculosis agents, aminosalicylates, antituberculosis
  combinations,diarylquinolines, hydrazide derivatives, miscellaneous
  antituberculosis agents, nicotinic acid derivatives, rifamycin
  derivatives, streptomyces derivatives
    antiviral agents
    adamantane antivirals
    antiviral boosters
    antiviral combinations
    antiviral interferons
    chemokine receptor antagonist
    integrase strand transfer inhibitor
    miscellaneous antivirals
    neuraminidase inhibitors
    NNRTIs
    NS5A inhibitors
    nucleoside reverse transcriptase inhibitors (NRTIs)
    protease inhibitors
    purine nucleosides
    carbapenems
    cephalosporins
    first generation cephalosporins
    fourth generation cephalosporins
    next generation cephalosporins
    second generation cephalosporins
    third generation cephalosporins
    glycopeptide antibiotics
    glycylcyclines
    leprostatics
    lincomycin derivatives
    macrolide derivatives
    ketolides
    macrolides
    miscellaneous antibiotics
    oxazolidinone antibiotics
    penicillins
    aminopenicillins
    antipseudomonal penicillins
    beta-lactamase inhibitors
    natural penicillins
    penicillinase resistant penicillins
    quinolones
    sulfonamides
    tetracyclines
    urinary anti-infectives
    antineoplastics
    alkylating agents
    anti-CTLA-4 monoclonal antibodies
    antibiotics/antineoplastics
    antimetabolites
    antineoplastic detoxifying agents
    antineoplastic interferons
    BCR-ABL tyrosine kinase inhibitors
    CD20 monoclonal antibodies
    CD30 monoclonal antibodies
    CD33 monoclonal antibodies
    CD52 monoclonal antibodies
    EGFR inhibitors
    hedgehog pathway inhibitors
    HER2 inhibitors
    histone deacetylase inhibitors
    hormones/antineoplastics
    miscellaneous antineoplastics
    mitotic inhibitors
    mTOR inhibitors
    multikinase inhibitors
    proteasome inhibitors
    VEGF/VEGFR inhibitors
    biologicals
    antitoxins and antivenins
    hematopoietic stem cell mobilizer
    in vivo diagnostic biologicals
    recombinant human erythropoietins
    cardiovascular agents
    agents for hypertensive emergencies
    agents for pulmonary hypertension
    aldosterone receptor antagonists
    angiotensin converting enzyme inhibitors
    angiotensin receptor blockers
    angiotensin receptor blockers and neprilysin inhibitors
    antiadrenergic agents, centrally acting
    antiadrenergic agents, peripherally acting
    antianginal agents
    antiarrhythmic agents
    group I antiarrhythmics
    group II antiarrhythmics
    group III antiarrhythmics
    group IV antiarrhythmics
    group V antiarrhythmics
    anticholinergic chronotropic agents
    antihypertensive combinations
    ACE inhibitors with calcium channel blocking agents
    ACE inhibitors with thiazides
    angiotensin II inhibitors with calcium channel blockers
    angiotensin II inhibitors with thiazides
    antiadrenergic agents (central) with thiazides
    antiadrenergic agents (peripheral) with thiazides
    beta blockers with thiazides
    miscellaneous antihypertensive combinations
    potassium sparing diuretics with thiazides
    beta-adrenergic blocking agents
    cardioselective beta blockers
    non-cardioselective beta blockers
    calcium channel blocking agents
    catecholamines
    diuretics
    carbonic anhydrase inhibitors
    loop diuretics
    miscellaneous diuretics
    potassium-sparing diuretics
    thiazide diuretics
    inotropic agents
    miscellaneous cardiovascular agents
    peripheral vasodilators
    renin inhibitors
    sclerosing agents
    vasodilators
    vasopressin antagonists
    vasopressors
    central nervous system agents
    analgesics
    analgesic combinations
    antimigraine agents
    cox-2 inhibitors
    miscellaneous analgesics
    narcotic analgesic combinations
    narcotic analgesics
    nonsteroidal anti-inflammatory agents
    salicylates
    anorexiants
    anticonvulsants
    AMPA receptor antagonists
    barbiturate anticonvulsants
    benzodiazepine anticonvulsants
    carbamate anticonvulsants
    carbonic anhydrase inhibitor anticonvulsants
    dibenzazepine anticonvulsants
    fatty acid derivative anticonvulsants
    gamma-aminobutyric acid analogs
    gamma-aminobutyric acid reuptake inhibitors
    hydantoin anticonvulsants
    miscellaneous anticonvulsants
    neuronal potassium channel openers
    oxazolidinedione anticonvulsants
    pyrrolidine anticonvulsants
    succinimide anticonvulsants
    triazine anticonvulsants
    antiemetic/antivertigo agents
    5HT3 receptor antagonists
    anticholinergic antiemetics
    miscellaneous antiemetics
    NK1 receptor antagonists
    phenothiazine antiemetics
    antiparkinson agents
    anticholinergic antiparkinson agents
    dopaminergic antiparkinsonism agents
    anxiolytics, sedatives, and hypnotics
    barbiturates
    benzodiazepines
    miscellaneous anxiolytics, sedatives and hypnotics
    cholinergic agonists
    cholinesterase inhibitors
    CNS stimulants
    drugs used in alcohol dependence
    general anesthetics
    miscellaneous central nervous system agents
    muscle relaxants
    neuromuscular blocking agents
    skeletal muscle relaxant combinations
    skeletal muscle relaxants
    coagulation modifiers
    anticoagulant reversal agents
    anticoagulants
    coumarins and indandiones
    factor Xa inhibitors
    heparins
    thrombin inhibitors
    antiplatelet agents
    glycoprotein platelet inhibitors
    platelet aggregation inhibitors
    protease-activated receptor-1 antagonists
    heparin antagonists
    miscellaneous coagulation modifiers
    platelet-stimulating agents
    thrombolytics
    gastrointestinal agents
    5-aminosalicylates
    antacids
    antidiarrheals
    digestive enzymes
    functional bowel disorder agents
    anticholinergics/antispasmodics
    chloride channel activators
    guanylate cyclase-C agonists
    peripheral opioid receptor antagonists
    serotoninergic neuroenteric modulators
    gallstone solubilizing agents
    GI stimulants
    H. pylori eradication agents
    H2 antagonists
    laxatives
    miscellaneous GI agents
    proton pump inhibitors
    genitourinary tract agents
    impotence agents
    miscellaneous genitourinary tract agents
    tocolytic agents
    urinary antispasmodics
    urinary pH modifiers
    uterotonic agents
    hormones
    5-alpha-reductase inhibitors
    adrenal cortical steroids
    corticotropin
    glucocorticoids
    mineralocorticoids
    adrenal corticosteroid inhibitors
    antiandrogens
    antidiuretic hormones
    antigonadotropic agents
    antithyroid agents
    aromatase inhibitors
    calcitonin
    estrogen receptor antagonists
    gonadotropin-releasing hormone antagonists
    growth hormone receptor blockers
    growth hormones
    insulin-like growth factor
    parathyroid hormone and analogs
    progesterone receptor modulators
    prolactin inhibitors
    selective estrogen receptor modulators
    sex hormones
    androgens and anabolic steroids
    contraceptives
    estrogens
    gonadotropin releasing hormones
    gonadotropins
    progestins
    sex hormone combinations
    somatostatin and somatostatin analogs
    synthetic ovulation stimulants
    thyroid drugs
    immunologic agents
    immune globulins
    immunostimulants
    bacterial vaccines
    colony stimulating factors
    interferons
    interleukins
    other immunostimulants
    therapeutic vaccines
    vaccine combinations
    viral vaccines
    immunosuppressive agents
    calcineurin inhibitors
    interleukin inhibitors
    other immunosuppressants
    selective immunosuppressants
    TNF alfa inhibitors
    metabolic agents
    antidiabetic agents
    alpha-glucosidase inhibitors
    amylin analogs
    antidiabetic combinations
    dipeptidyl peptidase 4 inhibitors
    incretin mimetics
    insulin
    meglitinides
    non-sulfonylureas
    SGLT-2 inhibitors
    sulfonylureas
    thiazolidinediones
    antigout agents
    antihyperlipidemic agents
    antihyperlipidemic combinations
    bile acid sequestrants
    cholesterol absorption inhibitors
    fibric acid derivatives
    miscellaneous antihyperlipidemic agents
    PCSK9 inhibitors
    statins
    antihyperuricemic agents
    bone resorption inhibitors
    bisphosphonates
    miscellaneous bone resorption inhibitors
    CFTR combinations
    CFTR potentiators
    glucose elevating agents
    lysosomal enzymes
    miscellaneous metabolic agents
    peripherally acting antiobesity agents
    urea cycle disorder agents
    miscellaneous agents
    antidotes
    antipsoriatics
    antirheumatics
    chelating agents
    cholinergic muscle stimulants
    illicit (street) drugs
    local injectable anesthetics
    miscellaneous uncategorized agents
    phosphate binders
    psoralens
    smoking cessation agents
    viscosupplementation agents
    plasma expanders
    psychotherapeutic agents
    antidepressants
    miscellaneous antidepressants
    monoamine oxidase inhibitors
    phenylpiperazine antidepressants
    selective serotonin reuptake inhibitors
    serotonin-norepinephrine reuptake inhibitors
    tetracyclic antidepressants
    tricyclic antidepressants
    antipsychotics
    atypical antipsychotics
    miscellaneous antipsychotic agents
    phenothiazine antipsychotics
    psychotherapeutic combinations
    thioxanthenes
    radiologic agents
    radiocontrast agents
    iodinated contrast media
    ionic iodinated contrast media
    lymphatic staining agents
    magnetic resonance imaging contrast media
    miscellaneous diagnostic dyes
    non-iodinated contrast media
    non-ionic iodinated contrast media
    ultrasound contrast media
    radiologic adjuncts
    cardiac stressing agents
    radiologic conjugating agents
    radiopharmaceuticals
    diagnostic radiopharmaceuticals
    therapeutic radiopharmaceuticals
    respiratory agents
    antiasthmatic combinations
    antihistamines
    antitussives
    bronchodilators
    adrenergic bronchodilators
    anticholinergic bronchodilators
    bronchodilator combinations
    methylxanthines
    decongestants
    expectorants
    leukotriene modifiers
    lung surfactants
    miscellaneous respiratory agents
    respiratory inhalant products
    inhaled anti-infectives
    inhaled corticosteroids
    mast cell stabilizers
    mucolytics
    selective phosphodiesterase-4 inhibitors
    upper respiratory combinations
    topical agents
    anorectal preparations
    antiseptic and germicides
    dermatological agents
    miscellaneous topical agents
    topical acne agents
    topical anesthetics
    topical anti-infectives
    topical anti-rosacea agents
    topical antibiotics
    topical antifungals
    topical antihistamines
    topical antineoplastics
    topical antipsoriatics
    topical antivirals
    topical astringents
    topical debriding agents
    topical depigmenting agents
    topical emollients
    topical keratolytics
    topical non-steroidal anti-inflammatories
    topical photochemotherapeutics
    topical rubefacient
    topical steroids
    topical steroids with anti-infectives
    mouth and throat products
    nasal preparations
    nasal antihistamines and decongestants
    nasal lubricants and irrigations
    nasal steroids
    ophthalmic preparations
    anti-angiogenic ophthalmic agents
    miscellaneous ophthalmic agents
    mydriatics
    ophthalmic anesthetics
    ophthalmic anti-infectives
    ophthalmic anti-inflammatory agents
    ophthalmic antihistamines and decongestants
    ophthalmic diagnostic agents
    ophthalmic glaucoma agents
    ophthalmic lubricants and irrigations
    ophthalmic steroids
    ophthalmic steroids with anti-infectives
    ophthalmic surgical agents
    otic preparations
    cerumenolytics
    miscellaneous otic agents
    otic anesthetics
    otic anti-infectives
    otic steroids
    otic steroids with anti-infectives
    sterile irrigating solutions
    vaginal preparations
    miscellaneous vaginal agents
    vaginal anti-infectives
Footnote: The above list of drug classes by the chemical type of the active ingredient or by the way it is used to treat a particular condition by indication can be modified by the reactive intermediates (Table I, and IB) are representative of the cations, radicals and anions that can be formed by the disclosure and that can react to produced products. Table data is referenced from literature.
```

Modified drugs from the above drug classes, Table IB by the invention can be used to treat disease indications given in Table IC below.

### Drugs for Disease and Condition

TABLE ID
Reactive Intermediates that may be generated and undergo reactions under
polarity reversal Electrolysis Conditions that undergo further reaction
to produce drugs for a particular disease or condition using one or more
drug classes.
Reactive Intermediates that may be generated and undergo reactions under
polarity reversal Electrolysis Conditions that undergo further reaction.

Most Common Diseases Conditions
Acne
ADHD
AIDS/HIV
Allergies
Alzheimer's
Angina
Anxiety
Arthritis
Asthma
Bipolar Disorder
Bronchitis
Cancer
Cholesterol
Colds & Flu
Constipation
COPD
Depression
Diabetes (Type 1)
Diabetes (Type 2)
Diarrhea
Eczema
Erectile Dysfunction
Fibromyalgia
Gastrointestinal
GERD (Heartburn)
Gout
Hair Loss
Hayfever
Heart Disease
Hepatitis A
Hepatitis B
Herpes
Hypertension
Hypothyroidism
Incontinence
IBD (Bowel)
Insomnia
Menopause
Mental Health
Migraine
Osteoarthritis
Osteoporosis
Pain
Psoriasis
Rheumatoid Arthritis
Schizophrenia
Seizures
Sexual Health
Stroke
Swine Flu
UTI
Weight Loss
Footnote: The above list of drug classes by the chemical type of the active ingredient or by the way it is used to treat a particular disease condition by indication can be modified by the reactive intermediates (Table I) are representative of the cations, radicals and anions that can be formed by the disclosure and that can react. Table 1D data is referenced from literature.

### Insecticides

An insecticide is a substance used to kill insects. Insecticides are used in agriculture, medicine, industry, and by consumers. Nearly all the insecticides have the potential to significantly alter ecosystems; many are toxic to humans; some concentrate along the food chain. Major classes of insecticides are organochlorides, organophosphates and carbamates, pyrethroids, neonicotinoids, ryanoids. The disclosed invention allows for the rapid modification of potential new insecticide candiates for optimum selection, or for the modification of existing insecticides to form derivatives and to reduce the side effects and retain most of the effectiveness. A non-limiting example would be the modification of Di-1,1-chlorophenyl-2,2,2-trichloroethane (DDT) that is used extensively worldwide as an insecticide for agriculture, despite its toxicity. DDT can be modified using the current invention to form derivatives that are less toxic, while retaining most of its effectiveness, by the proper selection of reagents. The invention is equally applicable to the other insecticides.

Given below are specific examples of insecticides containing —CCl₃, and —NH groups from DDT and imidacloprid, C₉H₁₀ClN₅O₂ that can be modified using the invention.

(Insecticide)-C—Cl₃+HOOC—CH₂—CH₂—OH→(Insecticide)-C—Cl₂—CH₂—CH₂—OH (New)
(Insecticide)-NH+HOOC—CH₂—CH₂—OH→(Insecticide)-N—CH₂—CH₂—OH (New)
In the above examples, the additive group or reactant was shown as HOOC—CH₂—CH₂—OH for simplicity and clarity, and non-limiting The additive group or reactant is more generally described as Y₁ or Y₂ in Table 1B, and may be selected based on the desired derivative.

### Chemical Grafting of Polymers

Grafting of polymers are carried out using functional groups that react chemically. These grafting reactions are not specific. The current invention of polarity reversal electrolysis and process allows for the grafting of side groups and functional groups to polymer backbones to create novel products. An example of such a reaction is given below.

Polycarboxylic acid alkali salt, Polymer-COOH→Polymer.
→Polymer+ —OCH₂CH₂—(CH₂CH₂O)ₙ—OCH₂CH₃
→Polymer-OCH₂CH₂—(CH₂CH₂O)n-OCH₂CH₃
Grafted Polyethylene oxide to poly carboxylic acid.

The polymer may contain —OH, —NH₂, —NH—, —SH, —CONH₂, —CO—NH—, or other functional groups that can be used for the grafting reaction. The grafting can yield grafted polymers that can be used as biomaterials, drug release agents, electrically activated actuators and prosthetics.

### Chemical Modification of Surfaces

Grafting of polymers to surfaces to modify surface properties are carried out using functional groups that react chemically. These grafting reactions are not specific. The current invention of polarity reversal electrolysis and process allows for the grafting of side groups and functional groups to polymer backbones on surfaces to create novel products with modified surface properties. An example of such a reaction is given below.

Polycarboxylic acid alkali salt, Surface Polymer-COOH→Surface Polymer.
→Surface Polymer+ —OCH₂CH₂—(CH₂CH₂O)ₙ—OCH₂CH₃
→Surface Polymer-OCH₂CH₂—(CH₂CH₂O)ₙ—OCH₂CH₃
Surface Grafted Polyethylene oxide to poly carboxylic acid

### Chemical Modification of Surfaces to Introduce Drugs for Controlled Release

Surfaces such as surfaces of catheters, medical devices and other surfaces that require controlled drug release can be grafted with drugs that may be released in a controlled manner. In such an application the device will be subjected to reverse polarity electrolysis in the presence to the drugs.

### P—Polymer for Polymer—Drug Conjugates

D-COOH═>D.+P—CH₂O.=>DOCH₂P Ether
D-OH═>DO.+P—CH₂CO.=>DOCOCH₂P Ester
D-OH═>DO.+P—CH₂.=>DOCH₂P Ether
The Drug-Ester link and Drug-Ether link will have different control release rates, and may be selected as desired for effectiveness.
Chemical Modification of Surfaces in Contact with Tissues and Blood for Biocompatibility.

Biomaterials are widely used as medical devices and as coatings for medical devices for biocompatibility. Compromises are made between biocompatibility and the functional physical properties of the device. The invention allows a medical device to be coated with a hard polymer that is nor biocompatible containing a few functional groups, and grafting a soft biocompatible polymer on top to achieve biocompatibility. Examples of medical devices are catheter, heart valve, stent, breast implant, dental implant, pacemaker, renal dialyzer, intraocular lens, contact lens, vascular graft and ventricular assist devices. For example an electrically conducting medical device made out of a metal or carbon, may be coated with a copolymer of methyl methacrylate or polylactic acid, and a few percent acrylic acid and subject to polarity reversal electrolysis in the presence of poly ethylene glycol or its or carboxy terminated polyethylene glycol. Possible characteristics of such modifications are: does not initiate immune response, controls cell adhesion and control water content, and tolerated by living organisms.

Polarity reversal electrolysis of the invention may be performed using a relatively low current density, medium current density or a high current density. If the current density is low, the frequency of the electrode polarity switch can be low. (If the current density is high, the frequency of the reverse polarity switch may need to be sufficiently high to produce hydrogen radicals that will react with the alkyl radicals and minimize Kolbe dimer formation.) Furthermore, the profile of the function generator of the polarity switch, sine wave, square wave or triangular wave or other function will determine the concentration of the reaction intermediates and the reaction products on and around the electrode at a particular instant. Typically, the electrolysis is performed using a current density of 0.002 to 4 A cm⁻². It is preferred that the current density be 0.01 to 1 A cm⁻², particularly 0.02 to 1.0 A cm⁻². The voltage can be high, as high as 250 V for high productivity, especially in the industrial scale. Usually it is preferred to employ a low voltage because of efficiency, equipment availability, heat transfer and safety reasons, for example less than 48V, particularly 3 to 15 V. The voltage may be chosen to achieve a balance between economy (at low voltage) and avoidance of by-products (at high voltages) and electrical efficiency and the symmetry of the polarity reversing function. As the voltage source, solar panels producing direct current can be used and avoid the use of inverters to convert alternating current to direct current and avoid the conversion losses, and allow the invention to be practiced in remote locations to produce pharmaceuticals, chemicals and other processes.

A relatively low or high current density may be achieved in any suitable way, for example by selecting appropriate electrode distance, electrolyte concentration and or cell voltage.

The anode and cathode of the apparatus used to perform electrolysis may be composed of materials that are the same as or different from one another, and each may be independently selected from carbon, natural graphite, synthetic graphite, conductive polymers, platinum, palladium, steel, copper, silver, gold, nickel, Ti/RuO₂ or any transition metal or transition metal compound, or other materials mentioned herein. In addition, catalysts can be deposited on the electrodes in order to enhance the electrolysis efficiency and product selection, such as the deposition of platinum, palladium and other transition metal catalysts on carbon electrodes. If the anode and/or cathode comprises carbon, then it is preferred that it comprise graphite or boron doped diamond.

In one embodiment of the invention, the anode is composed of a material other than graphite. In another embodiment of the invention both the anode and cathode are composed of the same material. In this embodiment it is preferred that they both comprise graphite.

In another embodiment of the invention the anode and cathode are composed of different materials. In this embodiment it is preferred that one of the materials comprise graphite.

The material of the electrodes is often critical, and the surface characteristics, the frequency of the switch and the type of function in the frequency switch will in general be important and critical. It is preferred that the electrodes have a rough surface, such as that provided by graphite rather than the smooth or glassy surface usually provided by, say, platinum. Porous electrodes with high internal surface areas are specifically preferred. This gives reaction intermediates the location and time to react at the surface or vicinity of the electrodes. A composite electrode could be provided having a highly conductive core of one material and a coating of a material of a suitable roughness and surface area. Also, a usually smooth material could be treated to produce the desired roughness and electro-catalytic activity to produce the desired product.

The anode and cathode of the apparatus in which electrolysis is to be performed may be arranged in such a manner that when they are placed in the solvent, the closest spacing between the anode and cathode in the solvent is from 0.1 to 10 mm. It is preferred that the closest spacing between the anode and cathode in solution is from 1 to 3 mm, in order to obtain high current densities and have sufficient space for the release of the products from the electrodes or for reactant flow in a flow through reactor. Multiple electrodes can be arranged so that the total surface area of the electrode can be increased to increase productivity.

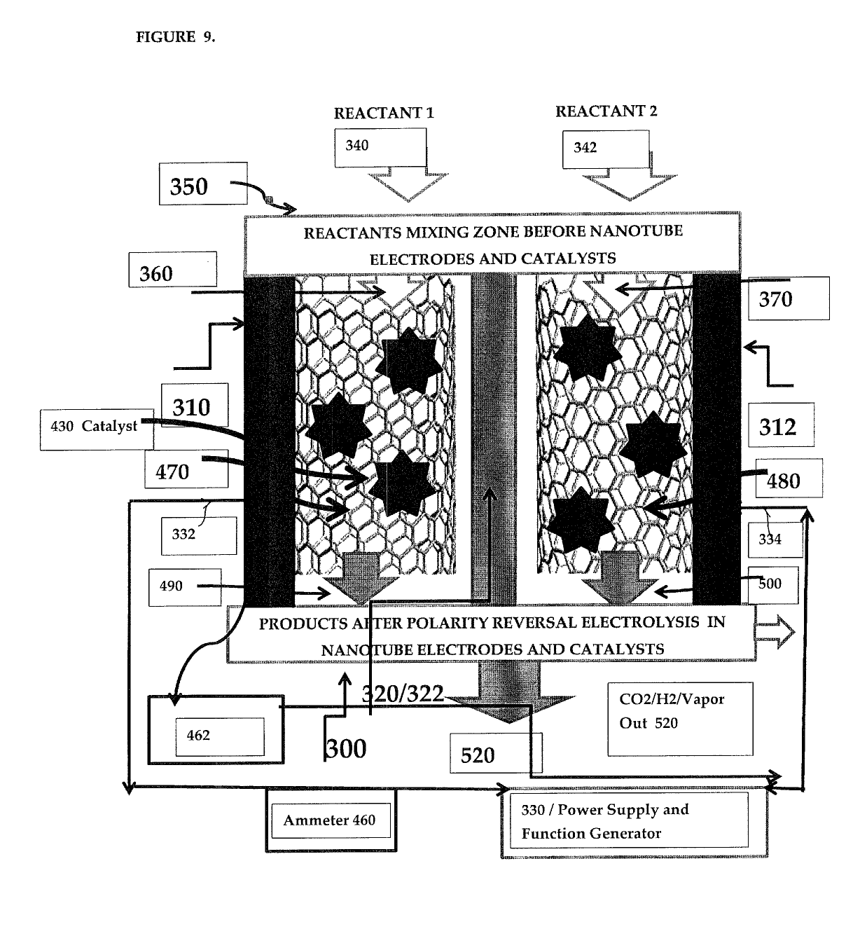

FIG. 9 is a description of the apparatus and process flow diagram illustrating a manufacturing process of the invention using electrodes that may be used in a flow through continuous process, wherein the said electrodes comprise carbon nanotubes, metal nanotubes, carbon nanofibers, metal nanofibers, carbon nanoparticles, metal nanoparticles, graphene, graphene oxide, graphite, polymer and combinations thereof. The carbon and metal nanotubes can be single walled, double walled or multiwalled carbon and metal nanotubes. One section of the nanotubes are in electrical contact with the positive port of the power supply and the other section, in contact with the negative port of the power supply, and the two sections of the electrode are electrically isolated. Electrical contact between the two electrodes is through the solution of the reactants and products. The mixture of reactants to be modified enters both entry ports of the nanotubes, and exits through the exits. While the reactants are inside the nanotubes, different reactive intermediates are generated inside the nanotubes during the anodic and cathodic cycle of the electrolysis. Upon polarity reversal, a different set of reactive intermediates are generated inside the nanotubes, and the reactions take place inside the nanotubes. Depending on the diameter of the nanotubes and the local concentration of the reactive intermediates and reactants, a high degree of chemical reactions will take place due to the space constraints, and will result in a more efficient reaction. The reaction products exit at the end of the carbon nanotubes.

The reaction conditions and degree of reaction inside the carbon nanotubes can be controlled by varying the applied voltage the frequency reversal and the use of a square wave, or other reversing wave. Furthermore, the nanotubes can contain catalyst nanoparticles to further enhance the reaction inside the nanoparticles. As catalyst nanoparticles, transition metals, transition metal oxides and other catalysts may be used. Transition metals and oxides from the First transition metal series, Scandium, Titanium, Vanadium, Manganese, Iron, Cobalt, Nickel, Copper, and Zinc as well as the transition metals and oxides from the second transition metal series from Yttrium, Zirconium, Niobium, Molybdenum, Technetium, Ruthenium, Rhodium, Palladium, Silver and Cadmium can be used. Furthermore, transition metals and oxides from the third transition metal series from Lanthanum, Hafnium, Tantalum, Tungsten, Rhenium, Osmium, Iridium, Platinum, Gold and Mercury can be used as selective catalysts. Transition metals and metal oxides of platinum, nickel, Raney nickel, manganese, rhodium, palladium, iron, vanadium and titanium are preferred.

An apparatus for accomplishing polarity-reversal electrolysis, comprising:

a reactor that comprises at least one pair of spaced electrodes, wherein the electrodes are made from a material selected from the group consisting of carbon nanotubes, metal nanotubes, single walled nanotubes, double walled nanotubes, multiwalled nanotubes, carbon nanofibers, metal nanofibers, carbon nanoparticles, metal nanoparticles, graphene, graphene oxide, graphite, polymer, and combinations thereof;

a polarity-reversing power supply that is adapted to provide polarity-reversed power to the electrodes; and a controller that controls at least the current, and the polarity reversal frequency, of the power supplied to the electrodes by the power supply so that the reaction between the reactants and intermediates inside the nanotubes can be changed by varying the voltage, reversal frequency and the characteristics of the voltage wave from a square wave, triangular wave or a sine wave.

The electrolysis step in the process of the invention is typically carried out on a solvent solution of a carboxylic acid, or salt or other derivative thereof, wherein the total concentration of the carboxylic acid and/or derivative in the solvent solution is usually around 2 molar, more usually at least 1 molar, for example about 1 molar. The precise value will often depend on the ability of a solvent to keep the material in solution. In the case where the reactants phase separates, the electrolysis reaction can be carried out under sonication or an externally generated emulsion by mechanical mixing such as by using a mechanical homogenizer or a fluidizer that generate emulsions by cavitation.

In principle any solvent or alcohol may be used as the solvent for the electrolysis process, provided that it is a liquid at the temperature at which the reaction is to be performed. It is preferred that the solvent dissolves the carboxylic acid, and the product alkane modified product is insoluble or sparingly soluble so that the product hydrocarbons phase separate from the reaction solution. This phase separation greatly facilitates the separation of the alkane or products from the reaction vessel by simple decantation from the solvent or by removal from the bottom, and makes the process an economically competitive separation process compared to separation by distillation and other methods. If the decarboxylated product is soluble in the solvent or solvent mixture, the product can still be separated by conventional distillation to recover the solvent and product.

Any solvent or solvent mixtures can be used for the process. Alkyl alcohols are more preferred, especially saturated, linear or branched C₁-C₅ alkyl alcohols. Alcohols that are particularly suitable include methanol, ethanol, n-propanol, i-propanol, n-butanol, s-butanol or t-butanol, ethylene glycol, polyethylene glycol, especially methanol, ethanol and n-propanol.

It is not essential for the solvent or alcoholic solution to be anhydrous. Up to 10% or more by volume of the solution may be water, more typically up to 8% by volume, and more preferably up to 4% by volume. In another embodiment, the solvent solution is anhydrous. In another embodiment the solvent is water or primarily water.

The solution of the fatty acid, or salt thereof, may comprise an alkali metal or alkaline earth metal hydroxide salt (especially LiOH, NaOH, KOH or in some situations Ca(OH)₂ although the latter material may have insufficient solubility in some solvents), or an amine salt from a tertiary, secondary, primary amine or an ammonium salt. A concentration of at least 0.5 M, preferably at least 1 M, particularly about 2 M will usually be suitable to achieve the desired current density, the metal ions and anions being the principle charge carriers during electrolysis. If a carboxylic acid is initially added to the solvent solution, then the alkali metal or alkaline earth metal hydroxide salt may be added to deprotonate the fatty acid in-situ. In one embodiment of the process, electrically conductive inorganic salts, particularly alkali metal (especially sodium and potassium) chlorides, sulfates, persulfates, perchlorates, carbonates and acetates are excluded from the solvent solution.

The electrolysis step generates heat and with heat the solvent solution may cause reflux of the solvent. It is preferred that electrolysis be performed at the reflux temperature of the solvent or the reactor immersed in a cold liquid bath or a jacketed reactor to remove the heat. It will usually be satisfactory to carry out the polarity reversing electrolysis at atmospheric pressure. In some situations, however, a high pressure might be desirable in order to allow a higher temperature to be used without excessive bubbling and for product selection and to increase the rate of reaction.

The process of the invention converts a carboxylic acid or salt thereof, into an alkane or alkene or a mixture of an alkane, and alkene or alkyl-aryl compound depending on the initial reactants used. An ether can be produced depending on the polarity reversal electrolysis conditions and solvent used. In addition some esters may also be produced. The term carboxylic acid refers to any organic compound, aliphatic, cyclic, heterocyclic, or aromatic, that contains a carboxylic acid that can be decarboxylated by this invention to produce the decarboxylated product. The term fatty acid as used herein refers to an organic compound having a single carboxylic acid attached to an aliphatic chain, which may be branched or unbranched and may be saturated or unsaturated. Typically, the fatty acid has at least 8 carbon atoms. The aliphatic chain of the fatty acid may be branched or unbranched, and typically the fatty acids are derived from triglycerides and lipids from oils and fats from plant and animal sources by hydrolysis using acids, bases or at high temperatures and pressures with water and steam and is known in the art.

Suitable unbranched saturated carboxylic acids include one or more of butanoic acid, pentanoic acid, hexanoic acid, heptanoic acid, octanoic acid, nonanoic acid, decanoic acid, undecanoic acid, dodecanoic acid, tridecanoic acid, dodecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, pharmaceuticals containing carboxylic acid or other anionic group.

Suitable monounsaturated fatty acids include one or more of cis-5-dodecenoic acid, myristoleic acid, palmitoleic acid, oleic acid, eicosenoic acid, erucic acid, and nervonic acid. Suitable polyunsaturated fatty acids include linoleic acid, alpha.-linoleic acid, linolenic acid, arachidonic acid, eicosapentaenoic acid, docosahexaenoic acid and drugs containing carboxylic acid or an anionic group.

The term salt of a fatty acid refers to the carboxylate salts of the fatty acid (e.g. sodium oleate). The counter cation to the carboxylate anion is typically an alkali metal cation, an alkaline earth metal cation, ammonium or alkylated ammonium (NR where each R is independently a C1-4 alkyl group). In particular, the counter cation is preferably selected from one or more of lithium, sodium, potassium, rubidium, and ammonium. More preferably, the counter cation is sodium or potassium.

In one embodiment of the invention, the use of alkali metal salts of propionic acid (particularly sodium propionate), caprylic acid (particularly potassium caprylate), lauric acid (particularly sodium laurate), myristic acid (particularly sodium myristate), oleic acid (particularly potassium oleate), stearic acid (particularly potassium stearate), tridecanoic acid (particularly potassium tridecanoate), pentadecanoic acid (particularly potassium pentadecanoate), heptadecanoic acid (particularly potassium heptadecanoate) can also be used depending on the desired physical properties.

In one embodiment, the fatty acid, or salt thereof, is unsaturated, more preferably is monounsaturated or polyunsaturated. Preferably, the fatty acid, or salt etc. thereof, is monounsaturated and has a double bond. More preferably, the fatty acid is derived from vegetable oils, animal fats, and waste oils containing high free acid content by hydrolysis that is generally known in the art of triglyceride and ester hydrolysis.

Analysis of experimental results reveals that alkanes, ethers, alkenes and cyclo-alkenes are formed during the reaction based on the reaction conditions used in a ratio based on the frequency, voltage and current density with straight chain fatty acids. This ratio varies significantly, however, depending on the fatty acids involved, as well as on the reaction conditions of the inventive steps and departs from the prior art expected Kolbe dimer product and Hofer-Moest process and product compositions.

If the product compounds of the electrolysis constitute a fuel, rather than act solely or mainly as a fuel additive or chemical, it is preferred that the alkanes, ethers, the alkenes, aryl-alkanes, or the ethers, alkanes and alkenes together constitute at least 15% particularly at least 40%, preferably at least 75%, and more preferably at least 90% by weight of the total fuel composition.

Thus, the invention provides a composition comprising an ether and an alkane compound represented by formula AB, ANu or BE as defined in Equation, (19), (20) and (23) above.

In particular, the amount of the alkane, the ether, or the amount of alkene, or the amount of ether plus the amount of alkene, present may be for example at least 20% by weight of the composition, preferably at least 30, 40, 50, 60, 70, 80, 90 or 95%.

In the use or in the composition of the invention, the fuel composition may include one or more of a lubricity additive, combustion improver, detergent, dispersant, cold flow improver, dehazer, demulsifier, cetane improver, antioxidant, scavenger or a pollution suppressant typically used in the industry.

The hydrocarbon or hydrocarbon chain can be derived from any suitable feed stocks, and in particular from any biomass feedstock or in any way from biomass. For example, the hydrocarbon or hydrocarbon chain can be derived from a saturated fatty acid, or salt or other derivative from plant and animal origin triglycerides.

The composition can be formed by a process including electrolysis. Moreover, it can be formed by a process further including catalysis to further change the properties to meet the specification of a particular use by further transformation or reformation.

The polarity switching electrolysis can be performed in a batch, semi-continuous, or continuous mode of operation.

The product may be a hydrocarbon, alkyl-aryl hydrocarbon, hydrocarbon-ether mix which may be subjected to one or more further processing steps including but not limited to distillation, catalysis and crystallization. Thus, the ether and the hydrocarbon may be further separated or purified and/or reacted. The result may be a pure hydrocarbon and/or pure ether useful as synthetic fuel components.

The core manufacturing process is preferably therefore a non-Kolbe electrolysis of fatty acid salts (for instance sodium, potassium), performed in solution in a solvent or a lower alcohol (methanol, ethanol, isopropanol etc.) using a simple electrolysis cell with, for example, two or more graphite electrodes with relatively small nominal spacing in between (about 2 mm) and medium to high current density (less than approximately 0.05-0.2 Acm⁻²) under near reflux conditions, where evaporation heat can be used to discharge excess heat created by the current involved. The current density may be increased from 0.01 to 2 Acm⁻² provided the heat can be removed by reflux or by cooling of the reactor, with minimal production of the Kolbe dimer at a high production rate.

It is believed that polarity reversing electrolysis has not previously been used directly to produce the alkane from the decarboxylation of a carboxylic acid. Acetic acid and formic acids have been used to produce alkanes by cross Kolbe electrolysis to create alkanes with fewer carbon atoms beyond the fatty acid. In fact, such a process is not used today to create any hydrocarbons or fuels at any significant scale, let alone biofuels due to the cost of acetic acid and formic acid. The formation of an alkane by cross Kolbe reaction with formic acid is uncertain. Also, very few hydrocarbons today are being created commercially at any scale from biomass feed stocks, except using gasification and Fischer-Tropsch processes, which work very differently from electrolysis and by high temperature catalytic decarboxylation using hydrogen gas.

Further technical details relating to preferred embodiments of the invention follow. An intermediate bio-fuel, lubricant or renewable chemical composition according to the present invention can have the following structures:

Ether, RCH₂CH₂OR′ (I)

Alkane, RCH₂CH₂—H (II)

Alkene, RCH₂═CH₂ (III)

Alcohol, RCH₂CH₂—OH (IV)

Alkyl-Aromatic, RCH₂CH₂Ar (V)

The residues R, R′, Ar can represent one or more selected from the group consisting of a single H as well as any branched or unbranched, saturated or unsaturated alkyl group including, but not limited to methyl, ethyl, n-propyl, iso-propyl, allyl, all 4 butyls, E- or Z-crotonyl, neo-pentyl, all possible isoprenyls, octyls, nonyls, decyls, undecyls, dodecyls, tridecyls, tetradecyls, pentadecyls, pentadecenyls, hexadecyls, heptadecyls, heptadecenyls and heptadecadienyls and aromatic groups and pharmaceuticals and drugs.

The alkyl chain R′ can be an alkoxyalkane or aryloxy, such as the phenoxy group, and can comprise one or more selected from a group consisting of H, methyl, ethyl, propyl/iso-propyl, allyl, and all isomers of butyl, butenyl, pentyl, pentenyl and hexyl or aromatic group.

Hydrocarbon compositions, aliphatic as well as aliphatic-aromatic are a main product of the core process. Ethers and alcohols are the other products of the core process. Both are formed in varying amounts. These ethers can be used together with those hydrocarbons as a novel fuel mixture, with properties similar to B20/50/80 (i.e. a 20/50/80% biodiesel/petroleum fuel mixture or sequence) while performing better (higher energy content, lower cold filter plugging point (CFPP), less aggressive solvent properties, etc.). In this case the core process need be the only process employed. When water is used as an additional reagent, alcohols are produced, represented by formula (IV), and can be used as specialty chemicals as well as fuel additives. When aromatic solvents or additives are used as an additional reagent, alkyl-aryl products are produced, represented by formula (V), and can be used as specialty chemicals as well as fuel additives and fuels.

Alternatively, these ethers can be seen as intermediates that can be refined further, for instance into hydrocarbons using a catalytic process. The resulting products may be “pure” hydrocarbons (i.e. having no more than traces of other compounds). This is possible for applications where fuels containing ethers are unappealing for whatever reason. If catalytic processing is not desirable for any reason, the hydrocarbon/ether mixture can also be separated by means of conventional distillation or other suitable means.

Currently, ethers are not commonly used in diesel type of formulations. The processes used to prepare ethers based on gasification are very different from the invented process in that, for example, gasification and associated processes used to form ethers cannot easily produce other, for example longer-chain, ethers. The mixed alkyl-aryls are expected to contain favorable fuel properties such as low freezing points and therefore can be used advantageously as fuels, especially jet fuels.

In addition, the invention produces hydroxyl compounds if water is used along with the other solvents that can be used as oxygenated fuels or chemicals such as fatty alcohols.

In contrast to prior art biodiesel formulations (the main renewable fuel for diesel engines) having two oxygen atoms per molecule, the present ethers preferably have only one oxygen atom per molecule, and thus have greater energy content. In other words, the energy density of prior art biodiesel fuel formulations is lower than that of the present biofuel formulations. Moreover, prior art biodiesel formulations have some undesirable properties, e.g. they act as solvents that attack rubber and other materials in engines, and they have a fairly high melting range (e.g. palm oil biodiesel without additives melts between 5 and 10.degree. C.). In contrast, the present biofuel having ethers as their only non-hydrocarbon component in general act as very mild solvents at best, and they generally have a much lower melting range than biodiesel made from the same feedstock. This results in the present biofuel melting at or below well below—instead of above—the freezing point of water.

Furthermore, the low oxygen content in the present biofuel helps making internal combustion burn more completely and thereby results in less toxic emissions due to a cleaner combustion. In addition, the low oxygen content fuels produced fuels with high octane numbers without undesirable material interaction properties.

The present fuel composition consisting mainly of hydrocarbons is also much closer to petroleum-based diesel fuel in terms of engine and fuel distribution network material compatibility as well as shelf life.

### Alkenes

The invention also relates to a hydrocarbon composition comprising any unsaturated hydrocarbon, derived from any fatty acid or from any renewable source, utilizing any of the above or below described processes with or without variations with at least one double bond with cis- or “Z-” configuration.

The invention also concerns a hydrocarbon composition comprising any hydrocarbon manufactured using any one of the above or below described processes from any fatty acid or fatty acid derivative sourced from any non-fossil feedstock, characterized by three to twenty-two carbon atoms with any number of double bonds.

A particularly useful group of hydrocarbons forms another part of the present invention: short, medium or long chain alkenes, having one or more double bonds, with either of the general formulae (VI):

R—CH═CH—R′ and R—CH═CH—(CH₂)n-CH═CH—R″ (VI)

where the double bond has a “cis” or “Z-” configuration, n and the length of the R groups preferably being such that the total number of carbon atoms is from 10 to 21, and R and R′ may themselves contain further unsaturation. In the examples, Oleic acid, CH₃(CH₂)₇CH═CH(CH₂)₇COOH was used.

Those proficient in the art will, after reading this specification, appreciate the significance of this group of compounds specifically emphasized here, i.e. unsaturated hydrocarbons having 10 to 21 carbon atoms and double bond(s) with cis- or “Z-” configuration. For example, mention may be made of heptadecenes of the general formula C₁₇H₃₄ or similar compounds with more than one double bond—at least one of which has cis-configuration—with similar properties. The above groups are found in oils and fats feed stocks. Furthermore, these compounds can have multiple uses as specialty chemicals.

These compounds differentiate themselves by the distinguished stereo chemistry of that particular middle double bond(s), which is always “cis” or “Z-” (same-sided), while the stereo chemistry of double bonds in refined petroleum feedstock is arbitrary in almost all cases. The hydrocarbons described immediately above, as well as those more generally described by formula (VI) can be directly derived by the manufacturing process that forms part of the invention. For instance, some members of the family of hydrocarbons in formula (VI) are unsaturated hydrocarbons with 17 carbon atoms, and can be derived from one particular unsaturated fatty acid, namely oleic acid, which is abundant in nature in both vegetable as well as animal fats and oils. The stereo chemistry of its double bond has a very well-defined configuration, practically 100% Z/cis, and the invented manufacturing process preserves this configuration after cleavage of the carboxyl group, reflected in the retained cis-, or “Z-”configuration of the hydrocarbons created. This is of tremendous advantage, as explained below.

This distinguished stereo chemistry leads to a certain preferred spatial molecular “bent” geometry of these compounds, which ultimately lowers their melting point (MP) significantly. This can also be observed in nature in many vegetable oils, which, despite having a fatty acid spectrum that is dominated by C₁₈ fatty acids, are liquid at room temperature. Conversely, animal fats with less oleic acid or other unsaturated fatty acids with cis- or “Z-”configuration are solid at room temperature. Furthermore, by using Alkyl-Aryl hydrocarbons as in formula V, the melting point can be further reduced, and would allow for the resulting alkyl-aryl hydrocarbons to be used as jet fuels as well as non-freezing renewable biofuels. In addition, this would allow for the use of unsaturated trans fatty acids such as elaidic acid, vaccenic acid and linoelaidic acid as well. Furthermore saturated fatty acids such as, palmitic acid, C15H₃₁COOH that generally have high melting points and is the most common saturated fatty acid found in animals, plants and microorganisms, is widely available and can be used for producing low-melting biofuels.

Hence hydrocarbons created from natural unsaturated fatty acids using the present process and having 17 or 15 carbon atoms melt far below zero or lower. At the same time, they are characterized by extremely low volatility and, hence, flammability (i.e. there is far less chance of igniting them accidentally during handling). This may be compared with straight heptadecane (C₁₇H₃₆), which has a melting point of 22.degree. C. (72.degree. F.), and would, on its own and without further refining, be practically unusable for diesel and, especially, jet fuel and aviation fuel.

Thus, the present C17 alkenes can provide an excellent blend stock for use with conventional diesel and jet fuel, and can even stand on their own and replace conventional diesel and jet fuel. Those of skill in the art will be aware that jet fuels require lower melting points than previously has been achievable with biofuels. Low melting points are required due to the extended stratospheric flights of jets, and accordingly, extended crossing through regions with very low temperatures.

### Alkyl-Aryl Coupling

In addition, the invention can be used for alkyl-aryl coupling by using the radicals carbocations produced by decarboxylation. In addition to using methanol, added water as a reactant, or using hexane as a solvent, aryl compounds and solvents can be used that are reactive to the generated radicals and carbocations during the reverse polarity electrolysis to produce alkylated aromatics.

The present embodiments in addition, teach a method to produce alkylated aromatics (AR) products which may, for example, be used as components in lubricants or as surface active agents. The properties of the formed AR products depend on the structure of both the alkyl and aryl components as well as the number of alkyl components that are coupled to a single aryl component. Common methods of preparing AR compounds are based on the Friedel-Crafts alkylation which uses a catalyst to alkylate aromatic compounds. Such a process can lead to the formation of monoalkylaromatics (MAR), dialkylaromatics (DAR) and polyalkaromatics (PAR). Because the properties of the MAR, DAR, and PAR may differ significantly from each other, a material with the desired properties is obtained by separating the different compounds through distillation and/or blending. One advantage of the present alkyl-aryl coupling of the aromatic component, or other component, is that by controlling the conditions and parameters of the electrolysis, one can control the degree of alkylation that occurs on the aromatic group, and thus control whether MAR products, DAR products, or PAR products are obtained and thus provides control over the properties of the synthesized compounds

In this embodiment the radical, the carbocation can act as an electrophile and subsequently involved in the electrophoretic substitution reaction. In such a reaction, the electrophile substitutes one of the substituents on an aromatic group, for example, hydrogen, instead of the hydrogen generated by the electro-reversal, as shown below as a non-limiting example with toluene.

R₁⁺+C₇H₈→C₇H₇R₁+H⁺ (28)

R₁.+C₇H₈→C₇H₇R₁+H. (29)

The H⁺ or the H. may then be consumed, further reacted, etc., in the reactor in the vicinity of the electrodes or in the bulk solution. In the embodiment shown above, benzene is shown as the aromatic solvent or additive, instead of water or n-hexane. Those skilled in the art will appreciate that other aromatic or non-aromatic organic solvent or additives may also be used, that will react with the radical or the carbocation.

There are a large number of inexpensive carboxylic acid substrates that are available to use as the alkyl component of the alkyl-aryl coupling product. These carboxylic acid substrates can be coupled to a large number of possible aromatic compounds. The abundance of inexpensive substrates enhances the ability to control and fine-tune the properties of the synthesized AR compound to match the specific needs of the lubricant application (or any other desired application). The length of the alkyl group may affect the physical properties of the material, such as pour point, viscosity index, and flash point. The substitution on the aromatic system may increase the pour point, the viscosity index, and the flash point. The aryl component of the alkyl-aryl compound may affect the thermo-oxidative stability of the formed compound (because the electron-rich aromatic portion of the molecule can scavenge radicals and disrupt oxidation processes).

### Manufacturing

A preferred manufacturing process will now be explained, by which, in accordance with the invention, renewable or non-fossil (i.e. not derived from fossilization) feed stocks may be converted into useful hydrocarbons, ethers, or a mix of the two.

R—CR′R″—O—R′″ (VII)

The carbon chain in formula (VII) is determined by the type of renewable feedstock being used, and it typically has a chain length between three and twenty-two, depending on the kind of fatty acids that is decarboxylated in the process. Furthermore, Aromatic groups such as toluene or benzene may also be used as desired to obtain the desired properties for R′, R″ or R′″. Moreover, those of skill in the art will appreciate that general melting and boiling ranges correspond to molecular mass. In other words, the choice of chain length is determined in practice by final product requirements (e.g. broad liquid temperature range, low flammability, etc.). The fatty acid feed stocks can be obtained by the hydrolysis of fats and oils as is known in the art.

### Fuels

The present invention relates to a composition that can be particularly used as a biofuel, the composition comprising one or more of an ether, alkyl-aryl hydrocarbon compound and a hydrocarbon or a hydrocarbon chain. The ether and the hydrocarbon are preferably in a useful ratio and mixed in liquid form at room temperature. Such a composition and also the compositions described below are particularly suitable as biofuel.

The ether and the hydrocarbon can be mixed in any suitable ratio, preferably from about 1:99 to about 99:1, preferably from about 10:90 to about 90:10, more preferably from about 20:80 to about 80:20, more preferably from about 30:70 to about 70:30, more preferably from about 40:60 to about 60:40 and more preferably about 50:50.

The described hydrocarbon-ether compositions can be used directly as fuel, lubricant, or they can be processed in a catalytic or other process using, for example, modified alumina (Al.sub.2O.sub.3) or similar catalysts at about 350-400.degree. for a specified time, to split the long alkyl off as alkene and to recycle the short-chain alcohol. This can, at the same time, be used to rearrange the long alkyl chain into something more branched using more sophisticated catalysts/conditions, such as those that the person skilled in the art will be aware of.

It is highly desirable to increase the branching of the long alkyl chain and hence lower the melting point of any resulting hydrocarbons longer than thirteen carbons (which have a melting point higher than desirable in a commercial product, especially for jet fuel and aviation fuel.

Those skilled in the art will appreciate that, by substituting ubiquitous fatty acids as starting material for a high-performance biofuel, use of the invention directly impacts the alternative use of dwindling supplies of fossil fuels. It will also be appreciated that, by producing carbon-neutral biofuels, use of the invention can directly impact the environment in a positive way by reducing or eliminating carbon emissions. Thus, the invention can preserve fossil fuels while also protecting the environment.

In short, the invented hydrocarbon-ether and hydrocarbon compositions are more similar to conventional petroleum products than existing biodiesel, whilst being advantageously derived from similar natural and renewable sources, and whilst minimizing emissions of fossil CO₂, i.e. whilst maintaining carbon neutrality.

Moreover, the ethers that are produced can, in accordance with one embodiment of the invention, be drawn off using suitable separation techniques, e.g. by fractionation techniques well known to those versed in the arts or any by other suitable process. These materials can stand on their own as biodiesel fuels or can be used as diesel fuel additives (e.g. to improve pour-point or cetane number, or to act as oxygenaters diminishing toxins in engine exhaust, etc.).

Uses for the present compositions include their applications as fuel and chemicals in any application where petroleum or products are used today. Thus, the present compositions may be similar to those in conventional use, but are made in a different way, from different sources, and have improved properties, e.g. the invented compositions may exhibit naturally ultra-low sulfur, estimated 90+% carbon-neutrality, etc.

### Lubricants and Chemicals

By the choice of the solvent or additives, lubricants and other chemical intermediates may be made by the above process. The choice is only limited by the selection of the reactants and the conditions of the polarity-reversal electrolysis.

The embodiments of the above recited patent application and invention are summarized further below.

### The Process

A process for the preparation of decarboxylated derivatives which comprises performing polarity reversing electrolysis using an anode and a cathode on a solvent of a carboxylic acid containing more than one carbon atom or salt of carboxylic acid or carboxylic acid ester or other derivative or precursor thereof, to decarboxylate said carboxylic acid or derivative, and produce the corresponding adduct hydrocarbons, alkanes, alkenes, alkyl ethers, alcohols and alkyl-aryl hydrocarbons. The polarity reversal electrolyses may be performed using a frequency range from 0.001 Hz to 3 MHz at a current density of 0.001 to 4.0 Acm⁻². The polarity reversal electrolyses may be performed using a polarity reversal voltage function selected from a sine wave, square wave or triangular wave at a frequency range from 0.001 Hz to 3 MHz at a current density of 0.001 to 4.0 Acm⁻² with a voltage range from 2 volt to 240 volts. The polarity reversal electrolyses may be performed using a polarity reversal voltage function that is symmetrical or unsymmetrical. The polarity reversal electrolyses may be performed using an anode and a cathode using materials comprising the same or different from one another selected from the group consisting of platinum, nickel, palladium, steel, copper, silver, gold, carbon, natural graphite, synthetic graphite or boron doped diamond.

The acid or carboxylic acid derivative may be selected from the group consisting of saturated or unsaturated aliphatic, aromatic, cyclic, heterocyclic acid or mixtures thereof. The carboxylic acid salt may be selected from the group consisting of an alkali metal, an alkaline earth metal salt, a salt formed by the alkali metal or alkaline earth hydroxide, a tertiary amine, a secondary amine, a primary amine or ammonia salt. The solvent may be selected from the group consisting of methanol, ethanol, propanol, isopropanol, butanol, pentanol, water, dimethyl sufoxide, acetonitrile, dimethyl formamide, formamide and N-methyl pyrollidone, aromatics, hexane or mixtures thereof.

The total concentration of the carboxylic acid or salt thereof in the alcoholic solution may be maintained to be between 0.1 and 4 molar. The solvent solution of the carboxylic acid or derivative thereof may be treated with and in contact with a tertiary amine, secondary amine, primary amine or ammonia. The solvent solution of the carboxylic acid or derivative thereof may be treated and in contact with an amine immobilized on a polymeric or silica support. The solvent solution of the carboxylic acid or derivative thereof may be treated and in contact with an alkali metal immobilized on a polymeric or silica support. The tertiary amine may be is selected from the group consisting of triethyl amine, diethyl cyclohexylamine, dimethyl cyclohexyl amine, piperidine, imidazole, benzoimidazole, and morpholine, or mixtures thereof.

The polarity reversal electrolysis may be performed between 0 degrees and 100 degrees Celsius. The polarity reversal electrolysis may be performed at substantially the reflux temperature of the solvent and solvent mixtures. The polarity reversal electrolysis may be performed between 1 bar and 100 bar pressure. The polarity reversal electrolysis may be performed in an apparatus having an anode and cathode wherein the closest spacing between the anode and cathode in the solvent is from 0.1 to 10 mm.

The carboxylic acid may be selected from the group consisting of saturated fatty acid, monounsaturated fatty acid, polyunsaturated fatty acid, aliphatic carboxylic acid, aromatic carboxylic acid, a derivative or precursor thereof, or mixtures thereof.

The process may comprise the step of separating the hydrocarbon from the solvent by phase separation. The process may comprise the step of separating the hydrocarbon from the solvent by distillation of the solvent. The process may comprise the step of separating the hydrocarbon from the solvent by freezing of the reaction mixture.

The carboxylic acid or derivative may be prepared by the hydrolysis of the triglyceride ester or the ester of the carboxylic acid. The triglyceride ester or ester may be derived from vegetable oils, animal fats, waste vegetable oils or waste animal fats. The hydrocarbon may be mixed with other hydrocarbons, alkyl-aryl hydrocarbons, hydrocarbon ethers or fatty acid esters or mixtures thereof to produce fuels, lubricants and chemicals. The hydrocarbon may be a saturated or an unsaturated alkane, an alkene, an alkyl-aryl hydrocarbon, an ether or an ester derivative.

The polarity reversing electrolysis may be carried out under vigorous mechanical mixing of the solution or under sonication in order to remove products away from the electrodes. The polarity reversing electrolysis may be carried out while the anode and cathode are subjected to mechanical vibration at 0.01 Hz to 100 kHz in order to remove reaction products and expose fresh electrode surfaces.

### Product by Process

Also featured is a product by process composition. The product may be of decarboxylated derivatives prepared by performing polarity reversing electrolysis using an anode and a cathode on a solution of a carboxylic acid containing more than one carbon atom or salt of carboxylic acid or carboxylic acid ester or other derivative or precursor thereof, to decarboxylate said carboxylic acid or derivative to produce the corresponding decarboxylated derivative. In addition, the solution may comprise solvents and additives that can react by radical coupling or by carbocation coupling with the solvent molecule or additives such as aryl additives, to form decarboxylated aryl compounds.

The carboxylic acid may be selected from the group consisting of a saturated or an unsaturated aliphatic, aromatic, cyclic, heterocyclic, fatty acid or mixtures thereof.

The product by process composition of the polarity reversal electrolysis may be generated using a polarity reversal voltage function selected from a sine wave, a square wave or a triangular wave at a frequency range from 0.001 Hz to 3 MHz at a current density of 0.001 to 4.0 Acm⁻² with a voltage range from 2 volt to 240 volts. The product by process composition of the polarity reversal electrolysis may be performed using a polarity reversal voltage function that is symmetrical or unsymmetrical. The product by process composition using a polarity reversal electrolysis process may be performed using an anode and a cathode comprising materials that are the same or different from one another, selected from the group consisting of platinum, nickel, palladium, steel, copper, silver, gold, carbon, natural graphite, synthetic graphite or boron doped diamond. Furthermore, the electrode surfaces may be coated with particles of platinum, nickel, palladium, copper, silver, gold and/or boron doped diamond, to catalyze the reaction. The above particles can be nanoparticles or micron-sized particles, or even a coating of the metals on internal surfaces of the electrodes. The coating of the metals can be performed by using electrolytic deposition of the metal ions or metal salts.

The product by process decarboxylated derivative may be further selected from the group consisting of saturated or unsaturated aliphatic, aromatic, cyclic, heterocyclic, or mixtures thereof. The carboxylic acid salt of the product by process invention may be selected from the group consisting of an alkali metal, an alkaline earth metal salt, a salt formed by the alkali metal or alkaline earth hydroxide, a tertiary amine, a secondary amine, a primary amine or ammonia salt. The solvent and solution for carrying out the product by process may be selected from the group consisting of methanol, ethanol, propanol, isopropanol, butanol, pentanol, water, dimethyl sufoxide, acetonitrile, dimethyl formamide, formamide and N-methyl pyrrolidone, aryloxy, alkoxy, hexane or mixtures thereof. In addition, additional reactants that can react with the decarboxylated radicals and carbocations, such as aromatic hydrocarbons and other compounds containing reactive groups, can be used to make novel adducts. The total concentration of the above carboxylic acid or salt thereof in the alcoholic solution or solvent is preferably maintained to be between 0.1 to 4 molar.

The solvent solution of the carboxylic acid or derivative thereof may be treated with and in contact with a tertiary amine, secondary amine, primary amine or ammonia during electrolysis. Furthermore, the amine can be immobilized on a polymeric or silica support for easy separation of the amine and the products. The tertiary amine may be selected from the group consisting of triethyl amine, diethyl cyclohexylamine, dimethyl cyclohexyl amine, piperidine, imidazole, benzoimidazole, and morpholine or mixtures thereof. The solvent solution of the carboxylic acid or derivatives can be treated and be in contact with an alkali metal immobilized on a polymeric or silica support.

The polarity reversal electrolysis may be performed between 0 degrees and 100 degrees Celsius, or performed at substantially the reflux temperature of the solvent. The polarity reversal electrolysis may be performed between 1 bar and 100 bar pressure. The polarity reversal electrolysis may be performed in an apparatus having an anode and cathode, and wherein the closest spacing between the anode and cathode in the solvent is from 0.1 to 10 mm.

Furthermore, the separation of the hydrocarbon and reaction products from the solvent may be performed by phase separation or by distillation of solvent. Furthermore, the hydrocarbon and reaction products may be separated from the solvent by freezing.

The carboxylic acid or derivative may be prepared by the hydrolysis of the triglyceride ester or ester of the carboxylic acid or other esters. The triglyceride ester or ester may be derived from vegetable oils, animal fats, waste vegetable oils or waste animal fats. The hydrocarbons produced by the product by process may be mixed with other hydrocarbons, alkyl-aryl hydrocarbons, hydrocarbon ethers or fatty acid esters or mixtures thereof to produce fuels, lubricants and chemicals. The hydrocarbon may comprise a saturated or an unsaturated alkane, alkyl-aryl, an alkene, an ether or an ester.

The polarity reversing electrolysis may be carried out under vigorous mechanical mixing of the solution or under sonication. The polarity reversing electrolysis may be carried out while the anode and cathode are subjected to mechanical vibration at 0.01 Hz to 100 kHz.

### Apparatus

Further disclosed is an apparatus for the preparation of decarboxylated derivatives which comprises performing polarity reversing electrolysis using an anode and a cathode using a polarity reversing device on a solution of a carboxylic acid containing more than one carbon atom or salt of carboxylic acid or carboxylic acid ester or other derivative or precursor thereof, to decarboxylate said carboxylic acid or derivative, by applying a voltage and current function sufficient to produce the corresponding decarboxylated hydrocarbon derivative. The polarity reversal electrolysis may be performed using a frequency range from 0.001 Hz to 3 MHz at a current density of 0.001 to 4.0 Acm⁻². The polarity reversal electrolysis may be performed using a polarity reversal voltage function selected from a sine wave, square wave or triangular wave at a frequency range from 0.001 Hz to 3 MHz at a current density of 0.001 to 4.0 Acm⁻² with a voltage range from 2 volt to 240 volts. The polarity reversal electrolysis may be performed in an apparatus using a polarity reversal voltage function that is symmetrical or unsymmetrical.

The polarity reversal electrolysis may be performed in an apparatus using an anode and a cathode using materials comprising the same or different from one another selected from the group consisting of platinum, nickel, palladium, steel, copper, silver, gold, carbon, natural graphite, synthetic graphite or boron doped diamond, or particles thereof.

The polarity reversal electrolysis may be performed in an apparatus containing a carboxylic acid or carboxylic acid derivative selected from the group consisting of saturated or unsaturated aliphatic, aromatic, cyclic, heterocyclic acid or mixtures thereof. The carboxylic acid salt in the polarity reversal electrolysis apparatus may be selected from the group consisting of an alkali metal, an alkaline earth metal salt, or a salt formed by the alkali metal or alkaline earth hydroxide, a tertiary amine, a secondary amine, a primary amine or ammonia salt. The solvent may be selected from the group consisting of methanol, ethanol, propanol, isopropanol, butanol, pentanol, water, dimethyl sufoxide, aromatic hydrocarbon, aryl-compounds, phenols, acetonitrile, dimethyl formamide, formamide and N-methyl pyrollidone, hexane or mixtures thereof. The total concentration of the carboxylic acid or salt thereof in the solution may be between 0.1 to 4 molar.

The solvent solution of the carboxylic acid or derivative thereof may be treated with and in contact with a tertiary amine, secondary amine, primary amine or ammonia. The solvent solution of the carboxylic acid or derivative thereof may be treated and in contact with an amine immobilized on a polymeric or silica support. The solvent solution of the carboxylic acid or derivative thereof may be treated and in contact with an alkali metal immobilized on a polymeric or silica support. The tertiary amine may be selected from the group consisting of triethyl amine, diethyl cyclohexylamine, dimethyl cyclohexyl amine, piperidine, imidazole, benzoimidazole, and morpholine or mixtures thereof.

The polarity reversal electrolysis may be performed at between 0 degrees and 100 degrees Celsius. The polarity reversal electrolysis may be performed at substantially the reflux temperature of the solvent. The polarity reversal electrolysis may be performed at between 1 bar and 100 bar pressure. The apparatus may have a closest spacing between the anode and cathode in the solvent that is from 0.1 to 10 mm.

The carboxylic acid may be selected from the group consisting of saturated fatty acid, monounsaturated fatty acid, polyunsaturated fatty acid, aliphatic carboxylic acid, aromatic carboxylic acid, a derivative or precursor thereof, or mixtures thereof.

The hydrocarbon may be separated from the solvent by phase separation, by distillation of the solvent, phase separation, or freezing.

The carboxylic acid or derivative may be prepared by the hydrolysis of the triglyceride ester or the ester of the carboxylic acid. The triglyceride ester or ester may be derived from vegetable oils, animal fats, waste vegetable oils or waste animal fats. The hydrocarbon may be mixed with other hydrocarbons, alkyl-aryl hydrocarbons, hydrocarbon ethers or fatty acid esters or mixtures thereof to produce fuels. The hydrocarbon may comprise a saturated or an unsaturated alkane, an alkene, an ether or an ester derivative.

The apparatus may further comprise a mechanical mixer or a sonicator wherein the polarity reversing electrolysis is carried out under vigorous mechanical mixing of the solution or under sonication. The apparatus may further comprise a mechanical vibrator, wherein the polarity reversing electrolysis is carried out while the anode and cathode are subjected to mechanical vibration at 0.01 Hz to 100 kHz.

This disclosure features a polarity-reversal electrolysis process, comprising providing a reactor that comprises at least one pair of spaced electrodes, providing a controlled polarity-reversing power supply that is constructed and arranged to provide polarity-reversed power to the electrodes, providing to the reactor an electrically-conductive liquid reaction medium that comprises reactants, wherein the electrodes are at least partially immersed in the reaction medium, and operating the power supply such that the polarity of the electrodes of the pair of electrodes reverses at a frequency rate. Also featured are products produced by the disclosed processes.

The reactor may comprise multiple separate pairs of spaced electrodes, each such pair supplied with polarity-reversed power by the power supply. The reactants may comprise a species that has an anion, and wherein the process produces a reactive radical intermediate at each electrode during the anodic cycle of each electrode. The reactants may comprise a species that has a carboxylic acid group, and wherein the process produces a decarboxylated radical intermediate at each electrode during the anodic cycle of each electrode.

The reactants may comprise a species that has a cation, and wherein the process produces a reactive radical intermediate at each electrode during the cathodic cycle of each electrode. The cation may comprise a hydrogen ion, and wherein the process produces a hydrogen radical intermediate at each electrode during the cathodic cycle of each electrode. The cation may comprise a species that has an alkali cation, or an alkali earth cation, and wherein the process produces an alkali metal radical intermediate at each electrode during the cathodic cycle of each electrode.

The process may produce a hydrogen radical at the cathode electrode during the cathodic cycle of each electrode. The process may produce carbonium ions at each electrode during the anodic cycle of each electrode. The process may produce carbanion ions at each electrode during the cathodic cycle of each electrode.

The spaced electrodes may comprise one or more materials selected from the group consisting of platinum, nickel, palladium, steel, copper, silver, gold, carbon, zinc, iron, chromium, titanium, transition metals, natural graphite, synthetic graphite, boron doped diamond and glassy carbon, or particles thereof.

The polarity reversal frequency may be from 0.001 Hz to 3 MHz. The current density may be from 0.001 to 4.0 Acm⁻². The voltage may be from 2 volts to 240 volts.

Also featured is an apparatus for accomplishing polarity-reversal electrolysis, comprising a reactor that comprises at least one pair of spaced electrodes, a polarity-reversing power supply that is adapted to provide polarity-reversed power to the electrodes, and a controller that controls at least the current, and the polarity reversal frequency, of the power supplied to the electrodes by the power supply. The reactor may comprise multiple separate pairs of spaced electrodes, each such pair supplied with polarity-reversed power by the power supply. The polarity reversal frequency may be from 0.001 Hz to 3 MHz. The current density may be from 0.001 to 4.0 Acm⁻². The voltage may be from 2 volts to 240 volts. The apparatus may further comprise at least one mechanism to stir the contents of the reactor. The apparatus may comprise a flow-through reactor. The space between the electrodes may be from 0.1 mm to 10 mm.

### DETAILED DESCRIPTION OF THE FIGURES

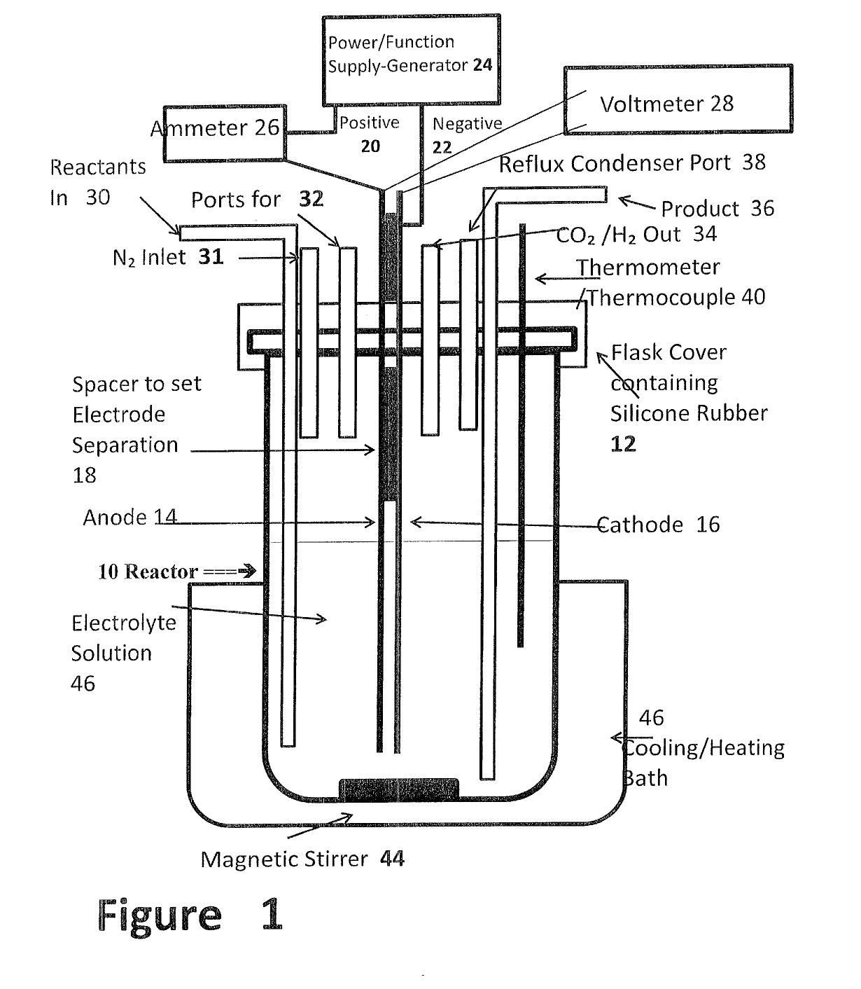

FIG. 1, describes the cylindrical reactor 10, with a silicone rubber sealable lid 12 for inserting the anode 14 and the cathode 16 that is separated by a fixed inert spacer 18 to maintain a fixed electrode separation between the electrodes. Leads, 20 and 22 from the power supply and function generator, 24, are connected to the two electrodes. The ammeter 26 is connected in series to measure current and the voltmeter 28 is connected to the electrodes, 14 and 16, to measure the applied voltage. Ports, 30, 31, 32 and openings, 34, 36 are provided or made as needed on the lid 12 for inserting a reflux condenser 38, ports for thermometers and thermocouples, 40, ports 34 for removing carbon dioxide and hydrogen generated during the reverse polarity electrolysis, ports for monitoring probes and sensors, 32, ports for introducing reactants 30, ports for removal of products 36, and ports 31 for introducing nitrogen or other inert gas as needed to flush the reactants. In addition, the reactor 10 is provided with a magnetic stirrer 44 for mixing the reactants during electrolysis. Additionally, the reactor can be inserted in a water or cooling bath 45 for reactor cooling and removing the heat of reaction. In addition, the reactor can be jacketed with a cooling jacket (not shown) for additional cooling if needed. This additional cooling most likely will be needed during scale up and manufacturing.

The port for product removal 36 allows for easy removal of final products. The port for reactants 30 allows for the introduction of fresh batch of reactants 46 that comprises the electrolyte solution. In addition to the batch mode operation, the apparatus described in FIG. 1 may be used in a semi-continuous mode.

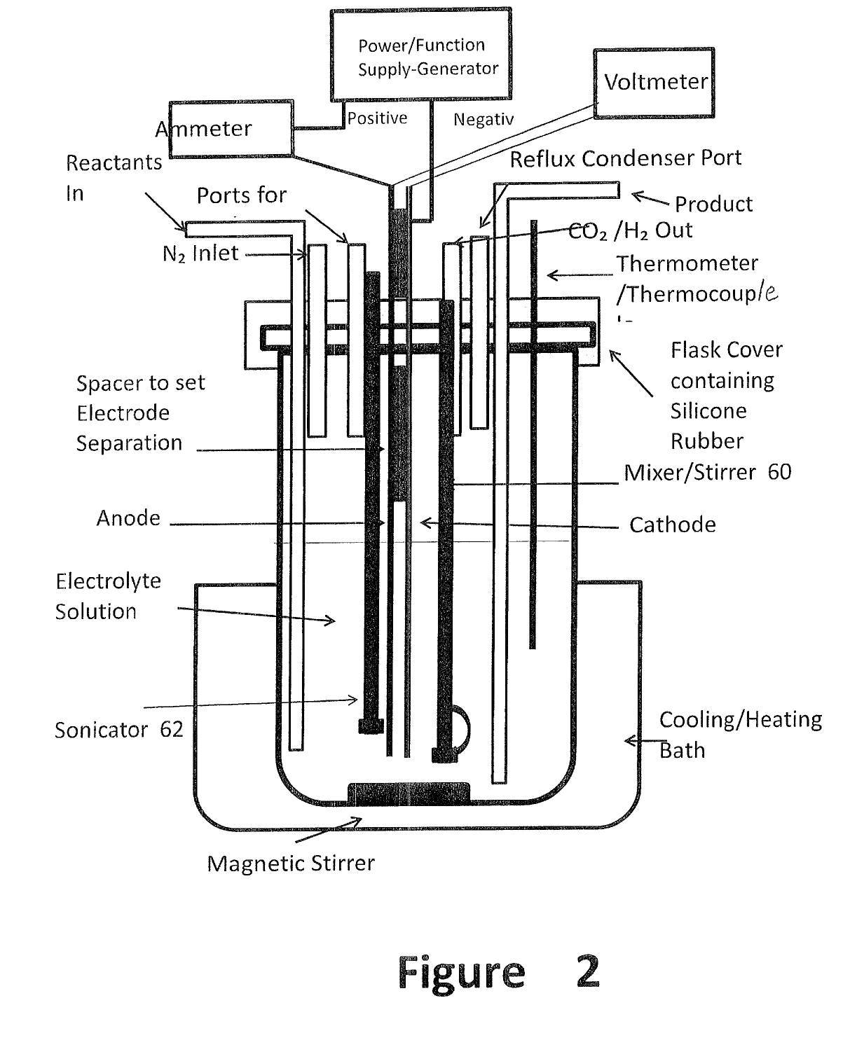

FIG. 2, describes the cylindrical reactor 10 described in FIG. 1, and in addition contains mechanical mixer-stirrer 60 and a sonicator 62 for additional product mixing, that will be useful during scale up and manufacturing. In addition, the reactor can be jacketed with a cooling jacket (not shown) for additional cooling if needed. This additional cooling most likely may be needed during scale up and manufacturing.

The port for product removal allows for easy removal of final products. The port 30 for reactants allows for the introduction of fresh batch of reactants. In addition to the batch mode operation, the apparatus described in FIG. 2 may be used in a semi-continuous mode

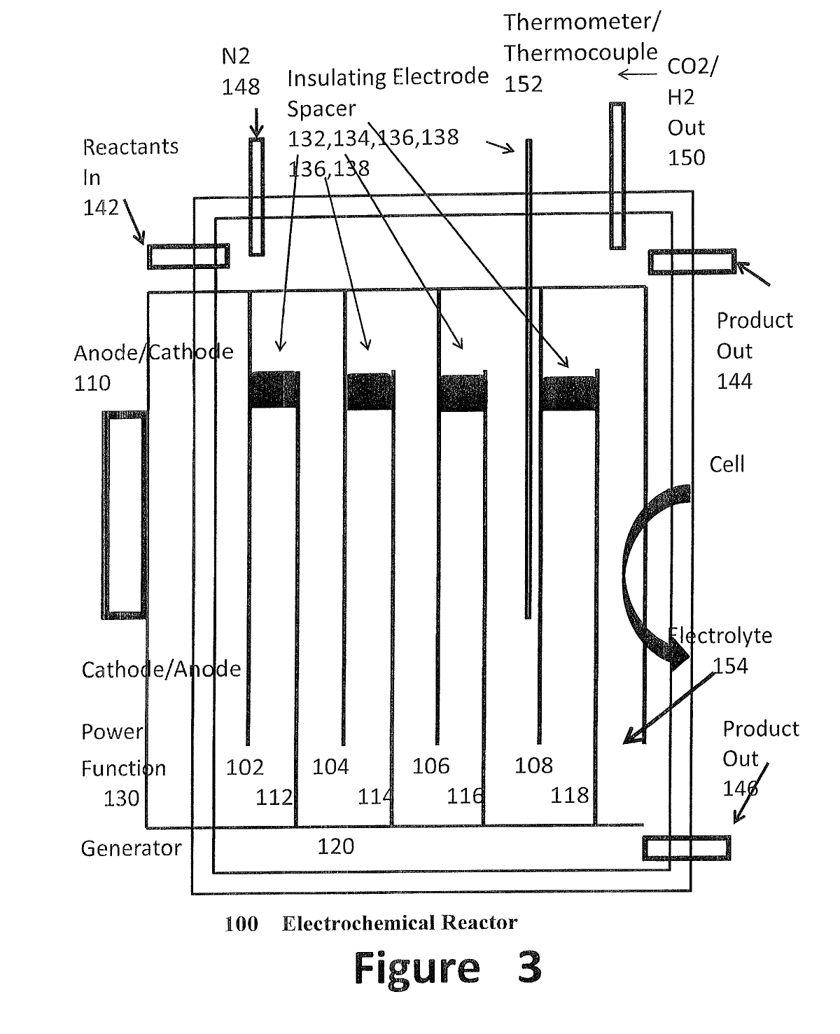

FIG. 3, describes a rectangular electrochemical reactor 100 containing multiple sets of anode-cathode pairs, 102-112, 104-114, 106-116,108-118, connected to the Power Generator/Function Generator 130 using the common leads 110 and 120, for carrying out the inventive process in a semi-continuous and continuous mode for scale up and manufacturing. Each of the anode-cathode electrode pairs are separated by insulating spacers, 132, 134, 136 and 138 and connected to a power supply and function generator 130 to provide the voltage and current necessary to carry out the reverse polarity reaction. Ports 142, 144 and 146 are provided for introducing in a continuous, semi-continuous or batch mode, the reagents and for removing the reaction products, in a continuous, semi-continuous or batch mode. Additional ports are provided for introducing any purging gases such as nitrogen 148 and for removing product gases 150 such as hydrogen, carbon dioxide and any other gases. Additional ports 152 are provided for thermometers, thermocouples and other sensors needed for monitoring the progress of the process. Additional cooling of the reactor may be provided by jacketing of the reactor (not shown). The reactant electrolyte 154 is contained in the reactor and immerses the electrodes.

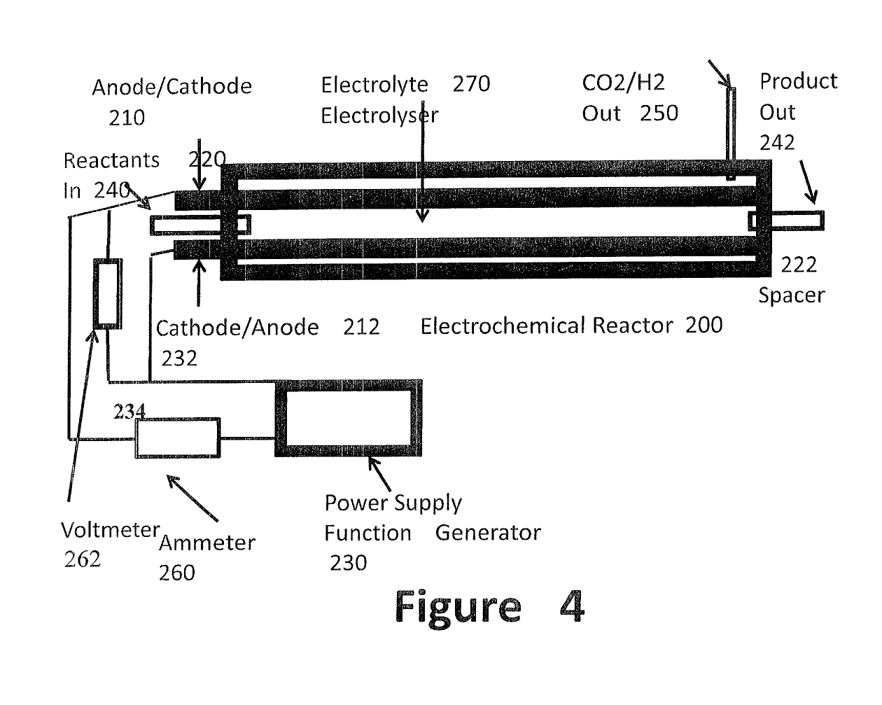

FIG. 4, describes a rectangular or tubular electrochemical reactor 200 containing a single set of anode-cathode pairs, 210 and 212, for carrying out the inventive process in a semi-continuous and continuous mode for scale up and manufacturing. The anode-cathode electrode pairs, 210 and 212, are separated by insulating spacers, 220 and 222 and connected to a power supply and function generator 230 using the leads 232 and 234 to provide the voltage and current necessary to carry out the reverse polarity reaction. Reactants enter through port 240 in a continuous, semi-continuous or batch mode, into the electrolysis chamber 270 between the electrodes and after the desired electrolysis and the reaction products exit, through port 242 in a continuous, semi-continuous or batch mode from the product out port, 242. An additional port 250 is provided for removing carbon dioxide and hydrogen. Further ports may be introduced as needed for monitoring the reaction conditions. An ammeter, 260, capable of measuring DC and AC current is connected in series, and a voltmeter, 262, capable of measuring DC voltage and AC voltage is connected to the anode and cathode, 210 and 212. The rate of reactant introduction will be determined by the rate of the electrolytic reaction and the variable used based in the desired outcomes.

FIG. 9 is a description of the apparatus and process flow diagram illustrating a manufacturing process of the invention using electrodes that may be used in a flow through continuous process, wherein the said electrodes comprise carbon nanotubes, metal nanotubes, carbon nanofibers, metal nanofibers, carbon nanoparticles, metal nanoparticles, graphene, graphene oxide, graphite, polymer and combinations thereof. The carbon and metal nanotubes can be single walled, double walled or multiwalled carbon and metal nanotubes. One section of the nanotubes are in electrical contact with the positive port of the power supply and the other section, in contact with the negative port of the power supply, and the two sections of the electrode are electrically isolated. Electrical contact between the two electrodes is through the solution of the reactants and products. The mixture of reactants to be modified enters both entry ports of the nanotubes, and exits through the exits. While the reactants are inside the nanotubes, different reactive intermediates are generated inside the nanotubes during the anodic and cathodic cycle of the electrolysis. Upon polarity reversal, a different set of reactive intermediates are generated inside the nanotubes, and the reactions take place inside the nanotubes. Depending on the diameter of the nanotubes and the local concentration of the reactive intermediates and reactants, a high degree of chemical reactions will take place due to the space constraints, and will result in a more efficient reaction. The reaction products exit at the end of the carbon nanotubes.

FIG. 9, describes a rectangular or tubular electrochemical reactor 300 containing a single set of anode-cathode pairs, 310 and 312, for carrying out the inventive process in a semi-continuous and continuous mode for scale up and manufacturing. The anode-cathode electrode pairs, 310 and 312, are separated by insulating spacers, 320 and 322 and connected to a power supply and function generator 330 using the leads 332 and 334 to provide the voltage and current necessary to carry out the reverse polarity reaction. Reactant 1 and reactant 2 enter through port 340 and 342 in a continuous, semi-continuous or batch mode, into the electrolysis mixing chamber 350 and enter each nanotube electrode through inlets 360 and 370, and enter the nanotube electrolysis chambers 470 and 480, and exit 490 and 500 to the products chamber, and exit through the product out port 520. Both the mixing chamber and the product chamber are in electrical contact through the liquid with the anode and the cathode, and the positive and negative terminals of the power supply. After the desired electrolysis, the reaction products exit, through port 520 in a continuous, semi-continuous or batch mode from the product out port. An additional port 550 is provided for removing any vapors and gases such as carbon dioxide and hydrogen. Further ports may be introduced as needed for monitoring the reaction conditions. An ammeter, 460, capable of measuring DC and AC current is connected in series, and a voltmeter, 462, capable of measuring DC voltage and AC voltage is connected to the anode and cathode, 410 and 412. The rate of reactant introduction will be determined by the rate of the electrolytic reaction and the variable used based in the desired outcomes. FIG. 9 also shows transition metal or metal oxide nanoparticle catalysts, 430, inserted inside the nanotubes for catalyzing the reactions which may be enhanced by catalysis. In FIG. 9, for clarity only two nanotubes were shown in the drawing describing the anode and cathode nanotubes. The nanotubes may range in diameter from 1 nm to 100 micrometers. The length of the nanometers may range from 10 nm to 1,000 micrometers. In practice, the nanotube electrodes comprise bundles of nanotubes embedded in an electrically conducting matrix, preferably a polymer matrix, separated by an insulating layer to electrically isolate the anode and cathode. This allows for scale up for product manufacturing. In addition, the anode ad cathode of the invention may comprise a conducting polymer matrix containing nanotubes, nanofibers, graphene, graphene oxide, graphite powders.

The process can also be used to react a mixture of gases or to dissociate a vapor or gas to carry out a transformation by the use of the catalysts and the electrical voltage and current that can be advantageously applied. For example methane has and water vapor or liquid can be used as the two reactants with the transition metal or metal oxide or other suitable catalyst to produce methanol and hydrogen. the proposed pathway for the electro catalytic conversion of methane and water to methanol and hydrogen.

At Electrode/Catalyst surface+H₂O→H⁺+.OH
At Electrode/Catalyst surface+H⁺→½H₂+.OH
CH₄+.OH→CH₃.+H₂O
CH₃.+H₂O→CH₃OH+½H₂
   If an additional reactant such as a chemical or drug is introduced t the methane water mix, the CH₃. radical produced can react with other reactive species generated by the added chemical, drug or pharmaceutical to be modified, and result in the introduction of a CH₃ group with the reactive centers and other radicals generated on the drug.

### Methylated, CH₃, for Methyl-Drug Conjugates

D-COOH═>D.+CH₃.=>DOCH₃ Methylated Drug
D-OH═>DO.+CH₃.=>DOCOCH₃ Ether Drug
D-SH═>DS.+CH₃.=>DSCH₃ Thioether Drug
Thus, the apparatus with nanotubes gives additional options for multiple reactions to be performed for the derivatization using reactive intermediates generated from a chemical, active pharmaceutical ingredient, pharmaceutical, drug, biologic, antibiotic, insecticide or antifungal at each electrode during the anodic cycle or the cathodic cycle. The methylation of the sites containing carboxyl, hydroxyl or sulfhydryl could change the pharmacological and toxicological properties of the drug derivative.

In the above Figures, the carboxylic acids partially or fully neutralized with the alkali metal hydroxides dissolved in the solvent is introduced into the electrolytic reactor containing the graphite or other electrodes and subjected to polarity reversing electrolysis, by applying the appropriate voltage using a function generator. The electrolytic current and applied voltage were measured. The electrolytic cell is cooled by using cold water or by allowing the solvent to reflux to remove the heat of the reaction and the heat generated by the electrical resistance. The current density is dependent on the electrical conductivity of the solution and the applied voltage, the electrode gap, the temperature and progress of the electrolysis. The reverse polarity function can be adjusted to meet the requirements of the desired reactions.

The Figures illustrate the present process in what is believed to be a largely self-explanatory process and apparatus. Pure fatty acid feedstock can be used, as indicated or produced by the hydrolysis of esters. Non-Kolbe polarity reversing electrolysis produces the novel and useful biofuels and chemicals of the invention, as described herein, typically including a hydrocarbon, ether or hydrocarbon alcohol mix. Alternatively, the novel biofuel produced by electrolysis undergoes separation, e.g. phase separation or fractionation to produce pure ethers, pure hydrocarbons, pure alkyl-aryl products or alcohols as desired. Those of skill in the art will appreciate that the ethers, hydrocarbons, and alcohols can be further processed into pure hydrocarbons, using any suitable process such as cleavage or catalysis, as is known in the art. The hydrocarbons can be used as diesel, jet fuel, aviation fuel, lubricants or similar chemical product, or can be conventionally or otherwise suitably refined to produce liquid propane gas, gasoline, or other desired chemical products. In addition, the process is very generic and can be used to produce different chemical intermediates and compositions by selecting the carboxylic acid, aliphatic, aromatic, cyclic, heterocyclic and produce new compounds and intermediate by free radical coupling and electrophilic reactions. The products of this invention can be difficult to produce chemicals, chemical intermediates and pharmaceutical intermediate and even new chemical entities that can be used as new drugs, that is difficult or uneconomical to synthesize.

For those skilled in the art, additional configurations can be constructed on order to optimize the inventive apparatus, the inventive process and variations in the product compositions.

### EXAMPLES

The invention will now be illustrated by the following, non-limiting examples. Gas chromatography/mass spectrometry was used to confirm production of a hydrocarbons, an alkene, and ether composition suitable for use as a biofuel and as chemicals. The fatty acids used in the examples below have been derived from naturally occurring vegetable oils. The examples are non-limiting in that any carboxylic acid can be substituted for the fatty acid, and can generally be produced by the hydrolysis of fats, oils and lipids. Oleic acid was used to reduce the invention to practice as oleic acid is a has both medical, nutritional and chemical uses, and is a major component of Mediterranean diets that helps in the prevention of breast cancer. Furthermore, oleic acid was used as it possess unsaturation, posses a cis configuration and an omega-9 monounsaturated fatty acid. The Table V describes the variety of substrate compounds including pharmaceuticals, drugs and other chemicals that may be used, and react of those can be used to replace oleic acid to practice the invention. The only limitation is the ability to generate reactive radicals, reactive radical anions, reactive radical cations, carbonium ions, carbanions suitable for further reaction by changing the voltage, functionality and frequency of the polarity reversal conditions.

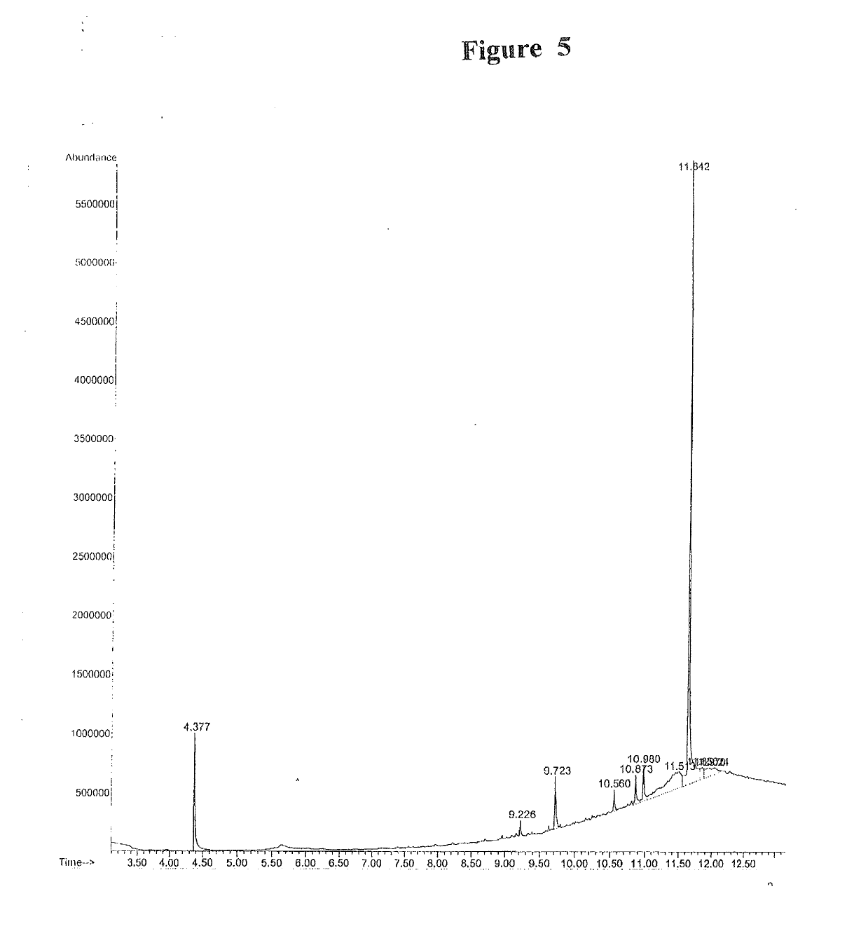

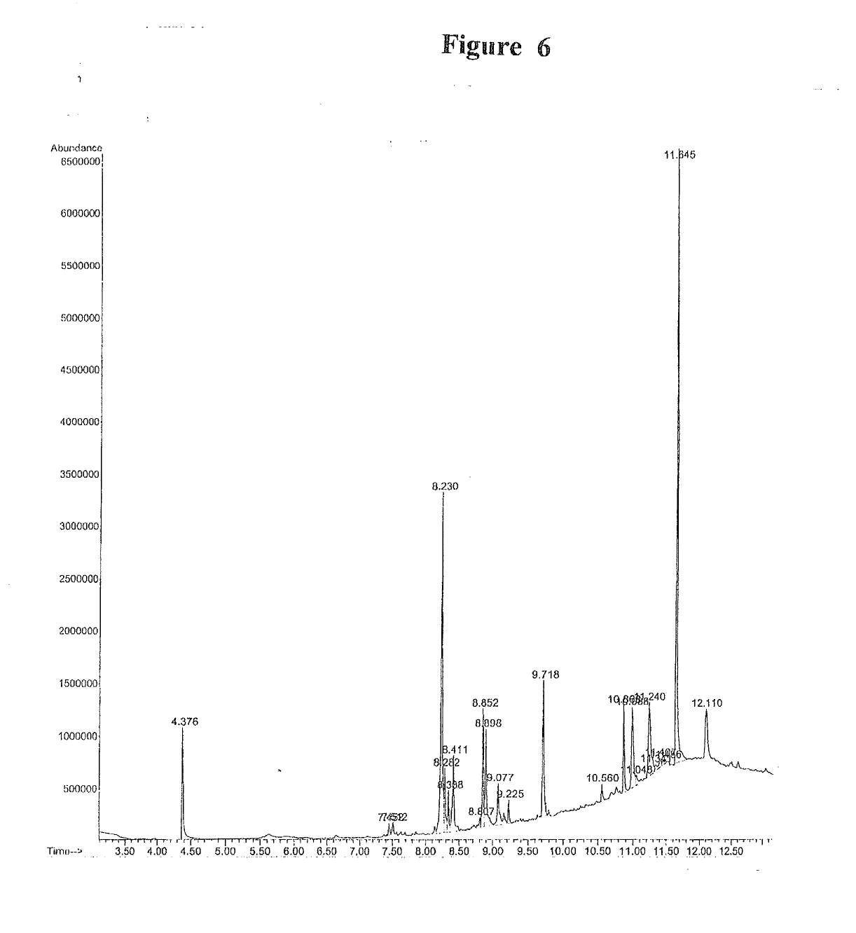

The control oleic acid, Laboratory Grade, Formula Weight 282.46, CAS 113-80-1, was obtained from Consolidated Chemical, Allentown, Pa. 18109, and used as received. The GC/MS analysis results of the control oleic acid is given in FIG. 5 and Table II. The predominant oleic acid methyl ester, 11-Octadecanoic acid methyl ester peak is at 9.72 min. Other peaks are impurities from the sample bottle and the lid of the sample bottle, especially the siloxanes, 2-butoxy ethanol and 13-Docosenamide, (Z) used as anti-static and release agents and was found in the GC/MS analysis. The oleic acid methyl ester was absent from the products to electrolysis, but new hydrocarbon peaks appeared as shown in FIGS. 6, 7 and 8 and Identified in Tables III, IV and V in substantial amounts from the invention. In GC/MS, the GC separates compounds based on retention time, and each peak may contain more than one compound. The MS identified each peak and assigns a probability value for the compound based on comparison to chemical databases. The results clearly demonstrate the utility and efficiency of reverse polarity electrolysis compared to the direct current electrolysis. More compounds are formed and it is expected that reactions can be controlled by adjusting the electrolysis reaction conditions.

TABLE II
GC/MS Results of Fatty Acid and Peak Identities from FIG. 5.

| | | FIG. 5 | | | |
|---|---|---|---|---|---|
| | | Fatty | | FIG. 5 | |
| | | Acid | | Fatty Acid | Peak |
| | GC-MS | Area % | FIG. 5 | Peak | Identifier |
| Peak | RT | RT, | Fatty Acid | Identifier | Quality |
| No. | min | Min | Peak Identifier | CAS # | Probability % |
| 1 | 4.378 | 9.86 | Ethanol; 2-Butoxyethanol | 000111-76-2 | 87, 72, 64 |
| 2 | 9.223 | 1.04 | Benzenepropionic acid, 3,5-Prop acid bis (1,1-dimethyl ethyl)-4-hydroxy-, | 006386-38-5 | 94 |
| | | | methyl ester) | | |
| 3 | 9.726 | 4.17 | 11-Octadecanoic Acid, methyl ester | 052380-33-3 | 99 |
| | | | 9-Octanedeconic acid (Z) methyl ester | 0000112-62-9 | 99 |
| | | | cis-13-Octadecenoic acid, methyl ester | 1000333-58-3 | 99 |
| 4 | 10.556 | 1.7 | Hexanedioic Acid, bis(2-ethylhexyl) ester | 000103-23-1 | 70 |
| | | | Diisooctyl Adipate | 0001330-86-5 | 64 |
| | | | Hexanedioic Acid, bis(2-ethylhexyl) ester | 000103-23-1 | 58 |
| 5 | 10.874 | 2.47 | 1,2-Bis(trimethylsilyl) benzene | 017151-09-6 | 86 |
| | | | Anthracene, 9,10-dihydro-9,9.10-trimethyl | 014923-29-6 | 53 |
| | | | 2-Ethylacridine | 055751-83-2 | 53 |
| 6 | 10.983 | 3.16 | 1,2-Bis(trimethylsilyl) benzene | 017151-09-6 | 41 |
| | | | 3′,8,8′-Trimethoxy-3-piperidyl-2,2′-binaphthalene-1,1′,4.4′-tetrone | 127611-84-1 | 38 |
| | | | Phthalic acid, 4-methoxyphenyl 2-propyl ester | 1000315-57-0 | 35 |
| 7 | 11.512 | 17.86 | Silane, 1,4-phenylenebis(trimethyl) | 013183-70-5 | 59 |
| | | | Trimethyl (4-2(2-methyl-4-oxo-2-pentyl) phenoxyl silane | 1000283-54-9 | 59 |
| | | | Cyclotrisiloxane, hexamethyl | 0000541-05-9 | 58 |
| 8 | 11.646 | 51.57 | 13-Docosenamide, (Z) | 000112-84-5 | 97 |
| | | | 13-Docosenamide, (Z) | 000112-84-5 | 94 |
| | | | 13-Docosenamide, (Z) | 000112-84-5 | 94 |
| 9 | 11.855 | 2.24 | 1,2 Bis(trimethylsilyl) benzene | 017151-09-6 | 64 |
| | | | Cyclotrisiloxane, hexamethyl | 000541-05-9 | 52 |
| | | | Cyclotrisiloxane, hexamethyl | 000541-05-9 | 52 |
| 10 | 11.973 | 3.6 | Trimethyl (4-2(2-methyl-4-oxo-2-pentyl) phenoxyl silane | 1000283-54-9 | 64 |
| | | | Silane, 1,4-phenylenebis(trimethyl) | 013183-70-5 | 64 |
| | | | 1,2-Bis(trimethylsilyl) benzene | 017151-09-6 | 41 |
| 11 | 12.023 | 2.34 | 1,2-Bis(trimethylsilyl) benzene | 017151-09-6 | 64 |
| | | | Cyclotrisiloxane, hexamethyl | 000541-05-9 | 52 |
| | | | 1,2-Benzenediol, 3,5-bis(1,1-dimethylethyl) | 001020-31-1 | 50 |
| | Total | 100.01 | | | |

### Example 1

### (B6-31) FIG. 6

### Oleic Acid to Hydrocarbons Using Polarity Reversal

To 102 g of a 50/50 mixture by weight of oleic acid (0.18M) and methanol in a bottle with a magnetic stirrer and a lid added 4.2 g of a 10% (w/w) solution of sodium hydroxide in methanol and mixed well with the magnetic stirrer. A control sample of 5 g solution was removed for analysis. A pair of graphite electrodes, (EDM1-Poco, Saturn Industries, New York) 2.5×15 cm and 1 mm thickness, separated by 2 mm, using polyethylene spacers was introduced to the bottle along with a thermocouple thermometer probe. The electrodes were immersed in the reactor such that 5 cm of the electrode pair was under the solvent solution. The electrodes were set so that the electrodes protruded through a silicone elastomer allowing for sealing the contents of the bottle as shown in FIG. 1. The electrodes were then connected to a DC power supply and the voltage increased from 1.25V to 14.95V with the polarity reversal set at 2.6 sec with a polarity reversal switch (E-mechanical timing relay, Allied Electronics, USA) and the current measured. The current increased from 0.097 A at 3.96 V to 0.39 A at 12.69V to 0.46 A at 14.95V. The voltage was then set to 12.69V and the polarity reversal switch was set to 0.6 sec. The initial temperature was 68 deg F. There was gas evolution from both electrodes and the temperature rose to 104 deg F within 50 minutes and the current increased to + and −0.48 A. The solution was clear. After 29 hrs the voltage was 12.46V and the current + and −0.36 A. After 34 hrs the voltage was 12.59 V and the current + and −0.19 A and there was gas evolution from both electrodes and the temperature 66 deg F. After 43 hrs, the temp was 66 deg F, voltage 12.59V and the current + or −0.15 A. There was a white precipitate at the bottom covering to about 6 mm and there was gas evolution with stirring. After 48 hrs, there was an oil layer at the bottom, height 1.8 cm with a diameter of 5.4 cm corresponding to 41 cubic cm of oil with greatly reduced production of gas bubbles. After 72 hrs, the voltage was 12.63V and the current + or −0.09 A and the electrolysis was stopped. The oil at the bottom was removed and 32.6 g of product oil was recovered. The measured yield from oleic acid from 50 g was 42.1 g, representing 79% of the theoretical yield. Balance was in the supernatant and as not reacted oleic acid. The supernatant was further electrolyzed, and the supernatant produced more oil that fell to the bottom of the electrolytic cell, and an additional 5 g of oil was recovered.

The initial fraction of product oil, was placed in a 40 ml glass bottle and placed in the freezer at −minus 12 deg C along with the control sample and oleic acid. The product oil was still liquid, oleic acid froze, and the control sample had at the bottom the frozen oleic acid and the methanol was at the top as a liquid.

The product Oil, Control Sample and Oleic Acid were analyzed by GC/MS. The results are given in FIG. 6.

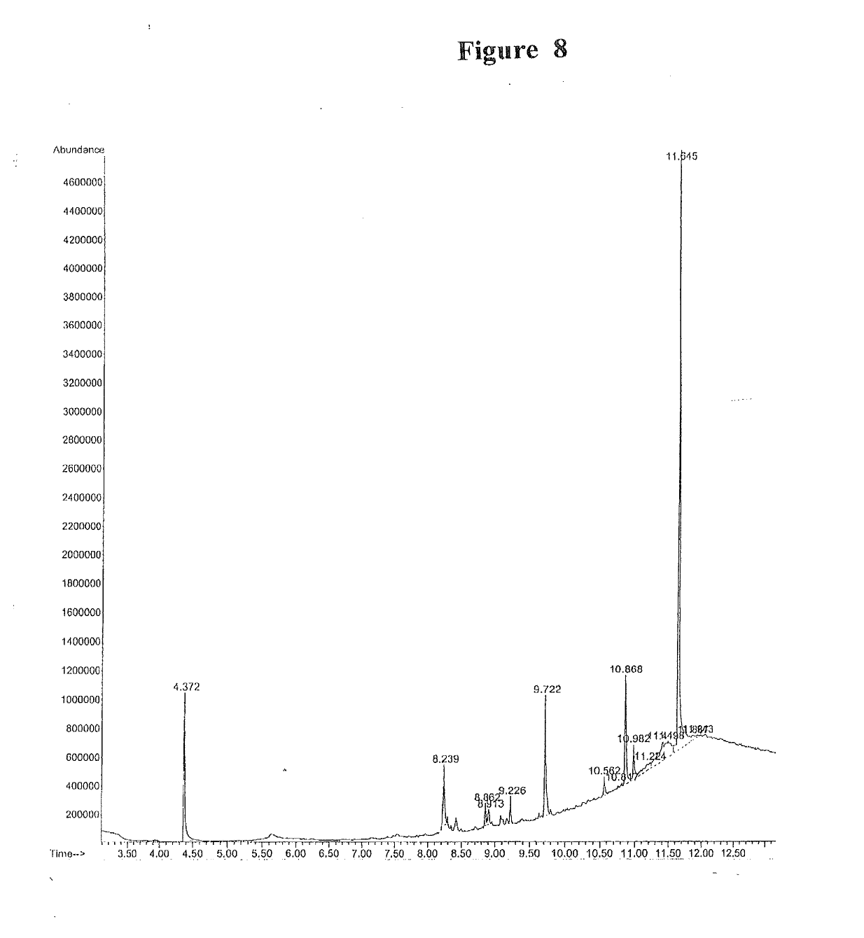

The analysis showed that part of the oleic acid had been transformed with multiple product peaks at 8.23, 8.85 and 8.89 min, that was not present in FIG. 5. When comparing the results with a Direct Current normal Kolbe and non-Kolbe results given in Example 4, FIG. 8, Table V, it shows that the amount of products with retention times of 8.23, 8.85 and 8.89 min were low for the normal Kolbe electrolysis. Additional compounds were formed that were not formed with the normal DC Kolbe electrolysis, Example 4, FIG. 8. Table I gives the GC/MS analysis by dissolving 0.1% by weight of the product in n-hexane analyzed by a third party independent analytical laboratory. The retention times are in minutes and the peak heights are normalized for the relative percentage of each component. Each retention time and each peak

TABLE III
GC/MS Results from Reverse Polarity Electrolysis and Peak Identities from FIG. 6.

| | | | | FIG. 5 | |
|---|---|---|---|---|---|
| | | FIG. 6 | | Fatty Acid | Peak |
| | GC-MS | B6- | FIG. 6 | Peak | Identifier |
| Peak | RT | 31 | B6-31 | Identifier | Quality |
| No. | min | Area % | Peak Identifier | CAS # | Probability % |
| 1 | 4.376 | 4.76 | Ethanol; 2-Butoxyethanol | 000111-76-2 | 91, 86, 72 |
| 2 | 7.455 | 0.63 | Cyclododecene | 001501-82-2 | 94 |
| | | | E-1,9-Tetradecadiene | 1000245-70-7 | 83 |
| | | | E-7-Dodecen-2-o1 = acetate | 1000131-35-3 | 80 |
| 3 | 7.513 | 0.61 | 1-Pentadecane | 013360-61-7 | 98 |
| | | | 1-Pentadecene | 013360-61-7 | 98 |
| | | | Cyclopentadecene | 000295-48-7 | 94 |
| 4 | 8.234 | 15.83 | E-1,9-Tetradecadiene | 1000245-70-7 | 96 |
| | | | Cyclododecene | 001501-82-2 | 94 |
| | | | cis-9-Tetradecen-1-01 | 035153-15-2 | 74 |
| 5 | 8.284 | 3.07 | 1,9-Tetradecadiene | 112929-06-3 | 87 |
| | | | Cyclododecene | 001501-82-2 | 76 |
| | | | cis-9-Tetradecen-1-01 | 035153-15-2 | 74 |
| 6 | 8.335 | 1.58 | Cyclododecene | 001501-82-2 | 89 |
| | | | 1,9-Tetradecadiene | 112929-06-3 | 76 |
| | | | cis-9-Tetradecen-1-01 | 035153-15-2 | 74 |
| 7 | 8.41 | 4.79 | Spiro(4,5) decane | 000176-63-6 | 87 |
| | | | 8-Hexadecyne | 019781-86-3 | 86 |
| | | | 9.12-Octadecadienoic acid (Z,Z) | 000060-33-3 | 80 |
| | 8.63 | | | | |
| 8 | 8.804 | 0.57 | Z,E-3,13-Octadecadien-1-ol | 1000131-10-4 | 83 |
| | | | E-2-Octadecadecen-1-ol | 1000131-10-2 | 81 |
| | | | Bicyclo(3.3.2) decan-9-one | 028054-91-3 | 78 |
| 9 | 8.854 | 4.68 | (4-Methyl-pent-3-enyl)-cyclohexane | 1000185-19-1 | 70 |
| | | | Bicyclo(2.2.2) octane, 2-methyl- | 1000131-10-2 | 55 |
| | | | 9,17-Octadecadienal, (Z) | 056554-35-9 | 47 |
| | 8.863 | | | | |
| 10 | 8.896 | 5.47 | (4-Methyl-pent-3-enyl)-cyclohexane | 1000185 = 19-1 | 38 |
| | | | 1,11-Dodecadiene | 005876-87-9 | 38 |
| | | | E-1,9-Hexadecadiene | 1000245-71-4 | 38 |
| | 8.913 | | | | |
| 11 | 9.081 | 2.34 | 1,11-Dodecadiene | 005876-87-9 | 95 |
| | | | Oleyl alcohol, methyl ether | 1000352-68-0 | 95 |
| | | | 1,13-Tetradecadiene | 021964-49-8 | 95 |
| 12 | 9.223 | 0.89 | Benzenepropionic acid, 3,5-Prop acid bis | 006386-38-5 | 94 |
| | | | (1,1-dimethyl ethyl)-4-hydroxy-, methyl ester) | | |
| | | | 8-(2,5-Dimethylanilino)naphtho-1,2-quinone | 1000058-06-6 | 46 |
| | | | 3,5-Di-tert-butyl-4-trimethylsiloxytoluene | 018510-49-1 | 40 |
| 13 | 9.718 | 6.27 | 8-Octadecanoic acid Methy; Ester | 002345-29-1 | 99 |
| | | | 11-Octadecenoic acid, methly ester | 052380-33-3 | 99 |
| | | | 9-Octadecenoic acid, methyl ester | 001937-62-8 | 99 |
| | 9.726 | | | | |
| | 10.556 | | | | |
| 14 | 10.564 | 0.62 | 1,2-Benzisothiazol-3-amine tbdms | 1000332-57-2 | 38 |
| | | | 1,2-Bis(trimethylsilyl) benzene | 017151-09-6 | 38 |
| | | | Silane, trimethyl (5-methyl-2-(1- | 055012-80-1 | 38 |
| | | | methylethyl)phenoxy)- | | |
| | 10.816 | | | | |
| 15 | 10.866 | 3.12 | Erucic acid | 000112-86-7 | 56 |
| | | | Fumaric acid, 2-chloropropyl dodecyl ester | 1000348-57-0 | 53 |
| | | | Erucic Acid | 000112-86-7 | 53 |
| | 10.874 | | | | |
| | 10.983 | | | | |
| 16 | 10.992 | 5.25 | Cycloheptadecanol | 004429-77-0 | 48 |
| | | | 9-Octadecenoic acid, (Z)-2,3 dihydroxypropyl ester methyl ester | 000111-03-5 | 45 |
| | | | 1-Cyclohexylnonene | 114614-84-5 | 41 |
| 17 | 11.051 | 0.63 | 1,2-Bis(trimethylsilyl) benzene | 017151-09-6 | 90 |
| | | | Silane, 1,4-phenylenebis(trimethyl) | 013183-70-5 | 81 |
| | | | Silane, trimethyl (5-methyl-2-(1- | 055012-80-1 | 53 |
| | | | methylethyl)phenoxy)- | | |
| | 11.227 | | | | |
| 18 | 11.243 | 6.17 | Benzene, 2-(tert-butyldimethylsilyl) oxy)-1-isopropyl-4-methyl- | 330455-64-6 | 38 |
| | | | 6-Octadecenoic acid(Z) | 000593-39-5 | 38 |
| | | | 9-Octadecenoic acid(Z)-,0-octadecenyl ester,)Z) | 003687-45-4 | 38 |
| 19 | 11.344 | 0.43 | Trimethyl (4-2(2-methyl-4-oxo-2-pentyl) phenoxyl | 1000283-54-9 | 59 |
| | | | silane | | |
| | | | 1,2-Bis(trimethylsilyl) benzene | 017151-09-6 | 53 |
| | | | Cyclotrisiloxane, hexamethyl | 000541-05-9 | 52 |
| 20 | 11.411 | 0.61 | Silane, 1,4-phenylenebis(trimethyl) | 013183-70-5 | 59 |
| | | | Cyclotrisiloxane, hexamethyl | 000541-05-9 | 58 |
| | | | Cyclotrisiloxane, hexamethyl | 000541-05-9 | 50 |
| 21 | 11.486 | 0.34 | 1,2-Bis(trimethylsilyl) benzene | 017151-09-6 | 59 |
| | | | Cyclotrisiloxane, hexamethyl | 000541-05-9 | 58 |
| | | | Cyclotrisiloxane, hexamethyl | 000541-05-9 | 58 |
| | 11.495 | | | | |
| | 11.512 | | | | |
| 22 | 11.646 | 26.92 | 13-Docosenamide, (Z) | 000112-84-5 | 98 |
| | | | 9-Octadecenamide, (Z) | 000301-02-0 | 97 |
| | | | 13-Docosenamide, (Z) | 000112-84-5 | 93 |
| | 11.855 | | | | |
| | 11.864 | | | | |
| | 11.939 | | | | |
| | 11.973 | | | | |
| | 12.023 | | | | |
| 23 | 12.107 | 4.41 | 1,2-Bis(trimethylsilyl) benzene | 017151-09-6 | 53 |
| | | | Silane, 1,4-phenylenebis(trimethyl) | 013183-70-5 | 50 |
| | | | Trimethyl (4-2(2-methyl-4-oxo-2-pentyl) phenoxyl silane | 1000283-54-9 | 47 |
| | Total | 99.99 | | | |

### Example 2

### B3-25

### Oleic Acid 60 Hz Sine Wave Electrolysis

To 104 g of a 50/50 mixture by weight of oleic acid (0.18M) and methanol in a reactor with a magnetic stirrer and a lid added 5 g of a 10% (w/w) solution of sodium hydroxide in methanol and mixed well with the magnetic stirrer. A control sample of 5 g solution was removed for analysis. A pair of graphite electrodes, 2.5×15 cm and 1 mm thickness, separated by 2 mm, using spacers was introduced to the reactor along with a thermocouple thermometer probe. The electrodes were immersed in the reactor and 5 cm of the electrode pair was under the solvent solution. The electrodes were set so that the electrodes protruded through a silicone rubber elastomer allowing for sealing the contents of the bottle as shown in FIG. 1. The electrodes were then connected to a AC power supply using a rheostat and the AC sine wave voltage increased to 32.5V AC. The AC current was 2.2 A. The temperature increased from 25 deg C to 66 deg C in 5 minutes. The power was turned off and the rheostat adjured to decrease the voltage to 14.5 V AC giving 1.02 A. The voltage was decreased to 11.5V and the current decreased to 0.75 A AC. There was no gas evolution from either electrode. The current decreased from 0.75 A AC to 0.3 A AC within 48 hrs and upon electrolysis for 7 days dropped to 0.05 A AC. However, the solution was clear and there was no oil at the bottom and there was no gas evolution. This result was different from the reverse polarity example 1.

The final product, of was placed in a 40 ml glass bottle and placed in the freezer at minus 12 deg C. along with the control sample and oleic acid. The product showed a precipitate, and the control sample had at the bottom the frozen oleic acid and the methanol was at the top as a liquid.

### Example 3

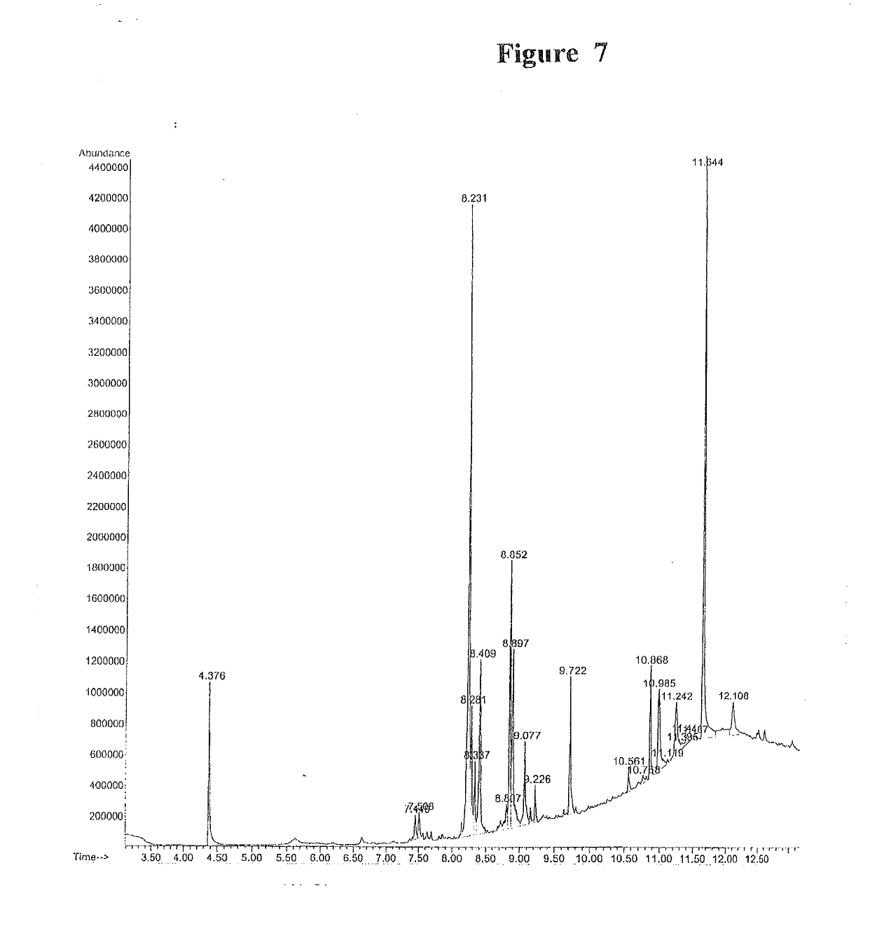

### (B5-49) FIG. 7

### Oleic Acid and Polarity Reversing Square Wave Electrolysis

To 100 g of a 50/50 mixture by weight of oleic acid (0.18moles) and methanol in a bottle with a magnetic stirrer and a lid was added 4.0 g of a 10% (w/w) solution of sodium hydroxide in methanol to provide a 1.4M solution of oleic acid and mixed well with the magnetic stirrer. A control sample of 4 g solution was removed for analysis. A pair of graphite electrodes, 2.5×15 cm and 1 mm thickness, separated by 2 mm using spacers was introduced to the bottle along with a thermocouple thermometer probe. The electrodes were immersed in the bottle such that 5 cm of the electrode pair was under the solvent solution with a gap for the magnetic stirrer. The electrodes were set so that the electrodes protruded through a silicone rubber outside the reactor allowing electrical connections and for sealing the contents of the reactor as shown in FIG. 1. The electrodes were then connected to a Function Generator Maetec Model SFG 1000 set at symmetrical square wave at 0.11 Hz, 20.5 Vpp. The voltage as measured at the electrode initially at the start of the electrolysis was +4.77 V and −4.69V and there was gas evolution at both electrodes. After 96 hrs there was a white gel at the bottom and the voltage was + and −7.36 V with oil drops floating. At 120 hrs, the white gel had turned into oil, and the oil layer was 1.5 cm thick. At 144 hrs, the voltage was + and −8.03 V and current + or −0.04 A and it was stopped at + or −8.04 volts with current + or −0.04 A. The oil layer at the bottom was 1.8 cm corresponding to a volume of 3.142×2.682.68×1.8 cubic cm or 38.2 ml of product oil or 29.8 g of product. The theoretical yield from 50 g of oleic acid, allowing for decarboxylation is 0.85*50=42.5 g. This gives a theoretical yield of 29.8/42.5, 70% yield, comparable to example 1 yield of 79% from example 1.

The recovered product oil from the bottom of the cell was removed and transferred into a 40 ml glass bottle and placed in the freezer at minus 12 deg C. along with the control sample and oleic acid. The product oil was still liquid, oleic acid froze, and the control sample had at the bottom the frozen oleic acid and the methanol was at the top as a liquid.

The product Oil, Control Sample and Oleic Acid were analyzed by GC/MS and the results are given in FIG. 7 and the peaks identified in Table IV.

The analysis showed that greater part of the oleic acid had been transformed with multiple product peaks at 8.23, 8.85 and 8.89 min, and at 8.41 min, with the height of the 8.23 min peak increasing substantially, that were not present in FIG. 5. When comparing the results with a Direct Current normal Kolbe and non-Kolbe results given in Example 4, FIG. 8, Table V, it shows that the amount of products with retention times of 8.23, 8.85 and 8.89 min were low for the normal Kolbe electrolysis. Additional compounds were formed that were not formed with the normal DC Kolbe electrolysis in this invention.

TABLE IV
GC/MS Results from Reverse Polarity Electrolysis and Peak Identities from FIG 7.

| | | | | FIG. 7 | |
|---|---|---|---|---|---|
| | | FIG. 7 | | B5 1-49 | Peak |
| | GC-MS | B5 1- | FIG. 7 | Peak | Identifier |
| Peak | RT | 49 | B5 1-49 | Identifier | Quality |
| No. | min | Area % | Peak Identifier | CAS # | Probability % |
| 1 | 4.378 | 5.01 | Ethanol; 2-Butoxyethanol | 000111-76-2 | 91, 91, 72 |
| 2 | 7.446 | 0.8 | Z-11,6-Y = Tridecadiene | 1000230-98-3 | 94 |
| | | | Z-1,8-Dodecadiene | 1000245-70-7 | 83 |
| | | | E-7-Dodecen-1-o1 acetate | 1000131-35-3 | 80 |
| 3 | 7.513 | 0.84 | 1-Pentadecane | 013360-61-7 | 98 |
| | | | 1-Pentadecene | 013360-61-7 | 98 |
| | | | Cyclopentadecene | 000295-48-7 | 94 |
| 4 | 8.234 | 21.47 | E-1,9-Tetradecadiene | 1000245-70-7 | 96 |
| | | | Cyclododecene | 001501-82-2 | 94 |
| | | | cis-9-Tetradecen-1-01 | 035153-15-2 | 74 |
| 5 | 8.284 | 4.19 | 1,9-Tetradecadiene | 112929-06-3 | 87 |
| | | | Cyclododecene | 001501-82-2 | 76 |
| | | | cis-9-Tetradecen-1-01 | 035153-15-2 | 74 |
| 6 | 8.335 | 2.06 | Cyclododecene | 001501-82-2 | 89 |
| | | | 1,9-Tetradecadiene | 112929-06-3 | 76 |
| | | | cis-9-Tetradecen-1-01 | 035153-15-2 | 74 |
| 7 | 8.41 | 7.12 | Spiro(4,5) decane | 000176-63-6 | 87 |
| | | | 8-Hexadecyne | 019781-86-3 | 86 |
| | | | 9.12-Octadecadienoic acid (Z,Z) | 000060-33-3 | 80 |
| | 8.63 | | | | |
| 8 | 8.804 | 1.06 | Z,E-3,13-Octadecadien-1-o1 | 1000131-10-4 | 83 |
| | | | E-2-Octadecadecen-1-o1 | 1000131-10-2 | 81 |
| | | | Bicyclo(3.3.2) decan-9-one | 028054-91-3 | 78 |
| 9 | 8.854 | 7.16 | (4-Methyl-pent-3-enyl)-cyclohexane | 1000185-19-1 | 70 |
| | | | Bicyclo(2.2.2) octane, 2-methyl- | 1000131-10-2 | 55 |
| | | | 9,17-Octadecadienal, (Z) | 056554-35-9 | 47 |
| | 8.863 | | | | |
| 10 | 8.896 | 7.59 | (4-Methyl-pent-3-enyl)-cyclohexane | 1000185 = 19-1 | 38 |
| | | | 1,11-Dodecadiene | 005876-87-9 | 38 |
| | | | E-1,9-Hexadecadiene | 1000245-71-4 | 38 |
| | 8.913 | | | | |
| 11 | 9.072 | 3.23 | 1,11-Dodecadiene | 005876-87-9 | 95 |
| | | | Oleyl alcohol, methyl ether | 1000352-68-0 | 95 |
| | | | 1,13-Tetradecadiene | 021964-49-8 | 95 |
| 12 | 9.223 | 0.97 | Benzenepropionic acid, 3,5-Prop acid bis | 006386-38-5 | 94 |
| | | | (1,1-dimethyl ethyl)-4-hydroxy-, methyl ester) | | |
| | | | 8-(2,5-Dimethylanilino)naphtho-1,2-quinone | 1000058-06-6 | 46 |
| | | | 3,5-Di-tert-butyl-4-trimethylsiloxytoluene | 018510-49-1 | 40 |
| 13 | 9.718 | 3.85 | 8-Octadecanoic acid Methy; Ester | 002345-29-1 | 99 |
| | | | 11-Octadecenoic acid, methly ester | 052380-33-3 | 99 |
| | | | 9-Octadecenoic acid, methyl ester | 001937-62-8 | 99 |
| | 9.726 | | | | |
| | 10.556 | | | | |
| 14 | 10.564 | 0.7 | 1,2-Benzisothiazol-3-amine tbdms | 1000332-57-2 | 38 |
| | | | 1,2-Bis(trimethylsilyl) benzene | 017151-09-6 | 38 |
| | | | Silane, trimethyl (5-methyl-2-(1-methylethyl)phenoxy)- | 055012-80-1 | 38 |
| 15 | 10.766 | 0.34 | | | |
| | 10.816 | | | | |
| 16 | 10.866 | 2.93 | Erucic acid | 000112-86-7 | 55 |
| | | | Erucic acid | 0000112-86-7 | 53 |
| | | | cis-10-Nonadecenoic acid | 073033-09-7 | 46 |
| | 10.874 | | | | |
| 17 | 10.984 | 4.25 | Phthalic acid, neopentyl 2-propyl ester | 1000315-56-3 | 25 |
| | | | Trimethyl (4-(2-methyl-4-oxo-2-pentyl) phenoxylsilane | 1000233-56-9 | 25 |
| | | | 1,2-Benzenedicarboxylic acid, diisooctyl ester | | 22 |
| | 10.992 | | | | |
| | 11.051 | | | | |
| 18 | 11.118 | 0.18 | 1,2-Bis(trimethylsilyl) benzene | 017151-09-6 | 59 |
| | | | Silane, 1,4-phenylenebis(trimethyl) | 013183-70-5 | 59 |
| | | | 5-methyl-2-phenylindolizine | 036944-99-7 | 52 |
| 19 | 11.243 | 3.34 | Cyclotrisiloxane, hexamethyl | 000541-05-9 | 46 |
| | | | Cyclotrisiloxane, hexamethyl | 000541-05-9 | 46 |
| | | | Trimethyl (4-tert-butylphenoxy) silane | 025237-79-0 | 43 |
| 20 | 11.336 | 0.34 | 2,4,6-Cycloheptatrien-1-one, 3,5-bis-trimethyl silyl- | 1000161-21-8 | 59 |
| | | | Trimethyl (4-2(2-methyl-4-oxo-2-pentyl) phenoxyl silane | 1000283-54-9 | 59 |
| | | | Trimethyl (4-2(2-methyl-4-oxo-2-pentyl) phenoxyl silane | 1000283-54-9 | 59 |
| | 11.344 | | | | |
| 21 | 11.411 | 0.55 | Trimethyl (4-(1-methyl-1-methoxyethyl)phenoxy silane | 1000283-54-8 | 59 |
| | | | 1,2-Bis(trimethylsilyl) benzene | 017151-09-6 | 59 |
| | | | Trimethyl (4-2(2-methyl-4-oxo-2-pentyl) phenoxyl silane | 1000283-54-9 | 59 |
| | | | Cyclotrisiloxane, hexamethyl | 000541-05-9 | 58 |
| | 11.486 | | | | |
| 22 | 11.487 | 0.34 | Trimethyl (4-(1-methyl-1-methoxyethyl)phenoxy silane | 1000283-54-8 | 59 |
| | | | 1,2-Bis(trimethylsilyl) benzene | 017151-09-6 | 59 |
| | | | Cyclotrisiloxane, hexamethyl | 000541-05-9 | 52 |
| | 11.495 | | | | |
| | 11.512 | | | | |
| 23 | 11.646 | 18.86 | 13-Docosenamide, (Z) | 000112-84-5 | 97 |
| | | | 9-Octadecenamide, (Z) | 000301-02-0 | 95 |
| | | | 13-Docosenamide, (Z) | 000112-84-5 | 93 |
| | 11.855 | | | | |
| | 11.864 | | | | |
| | 11.939 | | | | |
| | 11.973 | | | | |
| | 12.023 | | | | |
| 23 | 12.107 | 2.85 | Trimethyl (4-2(2-methyl-4-oxo-2-pentyl) phenoxyl silane | 1000283-54-9 | 64 |
| | | | Cyclotrisiloxane, hexamethyl | 000541-05-9 | 58 |
| | | | Cyclotrisiloxane, hexamethyl | 000541-05-9 | 52 |
| | Total | 100.03 | | | |

### Example 4

### Direct DC Electrolysis Control, FIG. 8

### Oleic Acid Direct Current Electrolysis—Normal Kolbe and Non-Kolbe Electrolysis

To 52 g by weight of oleic acid (0.18 molar) and 52 g methanol (1.70 molar) in a reactor with a magnetic stirrer and a lid added 4.4 g of a 10% (w/w) solution of sodium hydroxide in methanol and mixed well with the magnetic stirrer. A control sample of 5 g solution was removed for analysis. A pair of graphite electrodes, 2.5×15 cm and 1 mm thickness, separated by 2 mm, using spacers was introduced to the bottle along with a thermocouple thermometer probe. The electrodes were immersed in the bottle such that 5 cm of the electrode pair was under the solvent solution. The electrodes were set so that the electrodes protruded through a silicone rubber elastomer allowing for sealing the contents of the bottle as shown in FIG. 1. The electrodes were then connected to a direct current power supply and the voltage and current were measured. The voltage was then set at 12.6 volts, and the current was measured at 0.20 A and direct current electrolysis performed while the solution was stirred by the magnetic stirrer continuously. After 11 hrs, the DC voltage was 12.91V with and 0.03 to 0.04 A with gas bubbles from the electrodes. After 24 hrs, voltage was 13.1V and 0.01 A current with very little gas evolution. After 58 hours, the voltage was 13.03V with 0.01 A current with no gas evolution and phase separation with a clear solution. The final product was analyzed using GC/MS and the results are given in FIG. 8.

The final product, of was placed in a 40 ml glass bottle and placed in the freezer at −minus 12 deg C. along with the control sample and oleic acid. The product showed a precipitate, and the control sample had at the bottom the frozen oleic acid and the methanol was at the top as a liquid.

The final product was analyzed using GC/MS and the results are given in FIG. 8. A comparison of the results of FIG. 8, with FIGS. 7, 6 and 5 shows that reverse polarity non-Kolbe electrolysis produces more products and more efficient in converting oleic acid to other hydrocarbons and chemicals. Furthermore, the drastic drop in the current and the very slow reaction rates makes it impractical to use the direct current Kolbe and non-Kolbe electrolysis likely due to the coating of the electrodes with the products.

TABLE V
GC/MS Results from Direct Current Electrolysis and Peak Identities
from FIG. 8

| | | FIG. 8 | | FIG. 8 | Peak |
|---|---|---|---|---|---|
| | GC-MS | B7-1 | FIG. 8 | B7-1 DC | Identifier |
| Peak | RT | DC | B7-1 DC | Peak Identifier | Quality |
| No. | min | Area % | Peak Identifier | CAS # | Probability % |
| 1 | 4.37 | 9.56 | Ethanol; 2-Butoxyethanol | 000111-76-2 | 76, 72, 64 |
| | 7.446 | | | | |
| | 7.513 | | | | |
| | 8.234 | | | | |
| 2 | 8.243 | 4.46 | Z-1,9-Tetradecadiene | 100245-70-9 | 97 |
| | | | Cyclododecene | 001501-82-2 | 95 |
| | | | E-2-Octadecadecen-1-ol | 000506-42-3 | 93 |
| | 8.335 | | | | |
| | 8.41 | | | | |
| 3 | 8.63 | 1.44 | 1,13-Tetradecadiene | 021964-49-8 | 91 |
| | | | E-2-Methyl-3-tetradecadecen-1-ol acetate | 1000130-81-2 | 78 |
| | | | Bicyclo(2.2.2) octane, 2-methyl- | 000766-53-0 | 64 |
| | 8.804 | | | | |
| | 8.854 | | | | |
| | 8.863 | | | | |
| | 8.896 | | | | |
| 4 | 8.913 | 1.37 | 1,9-Tetradecadiene | 112929-06-3 | 64 |
| | | | (S) (+)-Z-13-Methyl-11-pentadecen-1-01 Acetate | 1000130-84-8 | 60 |
| | | | Z-8-Pentadecen-1-o1 acetate | 1000130-85-1 | 50 |
| | 9.072 | | | | |
| 5 | 9.223 | 1.52 | Benzenepropionic acid, 3,5-Prop acid bis | 006386-38-5 | 94 |
| | | | (1,1-dimethyl ethyl)-4-hydroxy-, methyl ester) | | |
| | | | Sarcosine, N-(3-phenylpropionyl)-isobutyl ester | 1000321-41-2 | 46 |
| | | | Cyclohexanone, 2-((1,1′-biphenyl)-2-ylamino) methylene) | 018510-49-1 | 38 |
| 6 | 9.718 | 7.88 | 8-Octadecanoic acid Methy; Ester | 002345-29-1 | 99 |
| | | | 9-Octadecanoic acid Methy; Ester-E | 052380-33-3 | 99 |
| | | | 9-Octadecanoic acid Methy; Ester-E | 001937-62-8 | 99 |
| | 9.726 | | | | |
| | 10.556 | | | | |
| 7 | 10.564 | 1.41 | 1,2-Bis(trimethylsilyl) benzene | 017151-09-6 | 42 |
| | | | Silane, 1,4-phenylenebis (trimethyl) | 013183-70-5 | 41 |
| | | | Cyclotrisiloxane, hexamethyl | 000541-05-9 | 38 |
| | 10.766 | | | | |
| 8 | 10.816 | 0.58 | Trimethyl (4-(2-methyl-4-oxo-2-pentyl) phenoxylsilane | 1000233-56-9 | 59 |
| | | | 1,2-Bis(trimethylsilyl) benzene | 017151-09-6 | 59 |
| | | | Methyltris(trimethsiloxy)silane | 017928-28-8 | 59 |
| 9 | 10.866 | 7 | Erucic acid | 000112-86-7 | 46 |
| | | | Erucic acid | 0000112-86-7 | 53 |
| | | | cis-10-Nonadecenoic acid | 073033-09-7 | 46 |
| | 10.874 | | | | |
| 10 | 10.984 | 2.77 | 1,2-Benzenedicarboxylic acid, mono(2-ethylhexyl) ester | 004376-20-9 | 38 |
| | | | 1,2-Benzenedicarboxylic acid, diisooctyl ester | 027554-26-3 | 38 |
| | | | 1,2-Bis(trimethylsilyl) benzene | 017151-09-6 | 38 |
| | 10.992 | | | | |
| | 11.051 | | | | |
| 11 | 11.227 | 2.63 | 2,4,6-Cycloheptatrien-1-one, 3,5-bis-trimethyl silyl- | 1000161-21-8 | 59 |
| | | | Silane, 1,4-phenylenebis(trimethyl) | 013183-70-5 | 59 |
| | | | 1,2-Bis(trimethylsilyl) benzene | 017151-09-6 | 59 |
| | 11.243 | | | 000541-05-9 | 46 |
| | | | | 000541-05-9 | 46 |
| | | | | 025237-79-0 | 43 |
| | 11.336 | | | | |
| | 11.344 | | | | |
| 12 | 11.411 | 5.77 | Silane, 1,4-phenylenebis(trimethyl) | 013183-70-5 | 59 |
| | | | 1,2-Bis(trimethylsilyl) benzene | 017151-09-6 | 59 |
| | | | Cyclotrisiloxane, hexamethyl | 000541-05-9 | 58 |
| | 11.486 | | | | |
| | 11.487 | | | | |
| 13 | 11.495 | 5.2 | Trimethyl (4-(1-methyl-1-methoxyethyl)phenoxy silane | 1000283-54-8 | 64 |
| | | | 5-Methyl-2-trimethylsilyloxy-acetophenone | 097389-69-0 | 59 |
| | | | Trimethyl (4-tert-butylphenoxy) silane | 025237-79-0 | 53 |
| | 11.512 | | | | |
| 14 | 11.646 | 47.32 | 13-Docosenamide, (Z) | 000112-84-5 | 97 |
| | | | trans-13-Octadecenamide | 010436-09-6 | 91 |
| | | | 13-Docosenamide, (Z) | 000112-84-5 | 89 |
| | 11.855 | | | | |
| 15 | 11.864 | 0.9 | 1,2-Bis(trimethylsilyl) benzene | 017151-09-6 | 59 |
| | | | 2,4,6-Cycloheptatrien-1-one, 3,5-bis-trimethyl silyl- | 1000161-21-8 | 59 |
| | | | 4-Methyl-2-trimethlysilyloxy-acetophenone | 097389-70-3 | 53 |
| 16 | 11.939 | 0.2 | Cyclotrisiloxane, hexamethyl | 000541-05-9 | 52 |
| | | | Trimethyl (4-2(2-methyl-4-oxo-2-pentyl) phenoxyl silane | 1000283-54-9 | 50 |
| | | | Cyclotrisiloxane, hexamethyl | 000541-05-9 | 50 |
| | 11.973 | | | | |
| | 12.023 | | | | |
| | 12.107 | | | | |
| | Total | 100.01 | | | |

### Example 5

### Polarity Reversing Electrolysis with Added Water

To 240 g of oleic acid added 184 g of methanol and mixed well using a magnetic stirrer and 60 g of a 10% w/w sodium hydroxide was then added slowly with mixing until the precipitated sodium salt dissolved. Distilled water, 15.3 gm, was added drop wise with stirring using a pipette to produce a clear solution stock solution 5.

60 g of this stock solution 5 was added to a 150 ml electrolysis bottle reactor containing a pair of graphite electrodes, 2.5×15 cm and 1 mm thickness, separated by 2 mm spacing and containing a thermocouple and a thermometer. The electrodes were immersed in the bottle such that 2.2 cm of the electrode pair was under the solvent solution with a gap for the magnetic stirrer. The electrodes were set so that the electrodes protruded through a silicon elastomer outside the bottle allowing electrical connections and for sealing the contents of the bottle as shown in FIG. 1. The electrodes were then connected to a polarity reversing switch with a timer that was fed by two sets of 30 Volt power supplies, set at 17.5V. The timer was set to change the polarity to the electrodes every 0.1 sec (10 Hz). The voltage as measured at the electrode initially at the start of the electrolysis measured as AC was 16.4 V, and the temperature rapidly rose from 83 d to 105 deg F, and there was gas evolution at both electrodes. The DC amps as measured at the power supply was 1.08 A and 1.10 A. After 36 hrs, the solution turned cloudy and the gas evolution between the electrodes, and at 40 hrs, there was phase separation with milky bottom layer and a cloudy top layer with the current at 0.46 amps. After 46 hrs, the current was 0.16 amps with the DC voltage at 17.3V, the measured AC voltage at 19.7V with 1.6 cm of white bottom phase and 0.8 cm of top cloudy supernatant phase. The yield of product oil from the bottom phase was 24.8 g.

The recovered product oil from the bottom of the cell was removed and transferred into three 15 ml centrifuge tubes, and placed in the freezer at minus 12 deg C. along with the control sample and oleic acid. The product oil froze unlike the product oil from the electrolysis in the absence of water.

### Example 6

### With Hexane as Additional Solvent

To 61 g of this stock solution 5, and 6 g of n-hexane were added to a 150 ml electrolysis bottle reactor containing a pair of graphite electrodes, 2.5×15 cm and 1 mm thickness, separated by 2 mm spacing and containing a thermocouple and a thermometer. The electrodes were immersed in the bottle such that 2.2 cm of the electrode pair was under the solvent solution with a gap for the magnetic stirrer. The electrodes were set so that the electrodes protruded through a silicon elastomer outside the bottle allowing electrical connections and for sealing the contents of the bottle as shown in FIG. 1. The electrodes were then connected to a polarity reversing switch with a timer that was fed by two sets of 30 Volt DC power supplies, and the voltage was gradually increased from 2V to 14V. Gas evolution started at around 8V. The timer was set to change the polarity to the electrodes every 0.1 sec (10 Hz). The voltage as measured at the electrode initially at the start of the electrolysis measured as AC was 16.4 V, and the temperature rapidly rose from 83 to 105 deg F, and there was gas evolution at both electrodes. The DC amps as measured at the power supply was 1.08 A and 1.10 A. After 36 hrs, the solution turned cloudy and the gas evolution between the electrodes, and at 40 hrs, there was phase separation with milky bottom layer and a cloudy top layer with the current at 0.46 amps. After 46 hrs, the current was 0.16 amps with the DC voltage at 17.3V, the measured AC voltage at 19.7V with 1.6 cm of white bottom phase and 0.8 cm of top cloudy supernatant phase. The yield of product oil from the bottom phase was 24.8 g.

The recovered product oil from the bottom of the cell was removed and transferred into three 15 ml centrifuge tubes, and placed in the freezer at minus 12 deg C. along with the control sample and oleic acid. The product oil froze unlike the product oil from the electrolysis in the absence of water.

### Method for Aryl-Alkyl Coupling Using Decarboxylation

In addition, the invention can be used for alkyl-aryl coupling by using the radicals produced by decarboxylation. In addition to using methanol, added water as a reactants, or using hexane as a solvent, aryl compounds and solvents can be used that are reactive to the generated radicals and carbanions during the reverse polarity electrolysis to produce alkylated aromatics and other alkyl-aryl compounds.

In this embodiment the radical, the carbocation can act as an electrophile and subsequently involved in the electrophoretic substitution reaction. In such a reaction, the electrophile substitutes one of the substituents on an aromatic group, for example, hydrogen, instead of the hydrogen generated by the electro-reversal, as shown below as a non-limiting example with benzene.

R₁⁺+C₆H₆→C₆H₅—R₁+

R₁.+C₆H₆→C₆H₅—R₁+H.

The H⁺ or the H. may then be consumed, further reacted, etc., in the reactor in the vicinity of the electrodes or in the bulk solution. In the embodiment shown above, benzene is shown as the aromatic solvent or additive, instead of water or n-hexane. Those skilled in the art will appreciate that other aromatic or non-aromatic organic solvent or additives may also be used, that will react with the radical or the carbocation.

TABLE VI
Summary of Reaction Conditions and Results

| | | Electrode Area 12.5 sq.cm | | | Electrode Separation 2 mm | | |
|---|---|---|---|---|---|---|---|
| | | Electrode | Electrode | | Electrolyzing | Electrolyzing | Electrolyzing |
| Electrolysis | | Reversing | Voltage | Electrolyzing | Current | Current | Current |
| Time | | Frequency, | Difference | Current | Density | Density | Density/V |
| Example 1 | | Cycles/sec, Hz | Volts | Amps | A/sq cm | mA/sq cm | mA/sq cm/V |
| | | Example 1 | 3.96 | 0.097 | 0.00776 | 7.76 | 1.960 |
| | | 0.40 Hz | 12.69 | 0.39 | 0.0312 | 31.2 | 2.459 |
| | | (2.6 sec/cycle) | 14.95 | 0.46 | 0.0368 | 36.8 | 2.462 |
| 50 | min | Square Wave | 12.69 | 0.48 | 0.0384 | 38.4 | 3.026 |
| 29 | hrs | | 12.4 | 0.36 | 0.0288 | 28.8 | 2.323 |
| 24 | hrs | | 12.59 | 0.15 | 0.012 | 12 | 0.953 |
| 72 | hrs | | 12.63 | 0.09 | 0.0072 | 7.2 | 0.570 |
| 5 | min | Example 2 | 32.5 | 2.2 | 0.176 | 176 | 5.415 |
| | | 60 Hz | 14.5 | 1.02 | 0.0816 | 81.6 | 5.628 |
| | | 0.0167 sec/ | 11.5 | 0.75 | 0.06 | 60 | 5.217 |
| | | cycle) | | | | | |
| 48 | hrs | Sine Wave | 11.5 | 0.3 | 0.024 | 24 | 2.087 |
| 7 | days | | 11.5 | 0.05 | 0.004 | 4 | 0.348 |
| 1 | hr | Example 3 | 4.77 | 0.05 | 0.004 | 4 | 0.839 |
| 96 | hrs | 0.11 Hz | 7.36 | 0.05 | 0.004 | 4 | 0.543 |
| 120 | hrs | 9.1 sec/cycle | 7.5 | 0.05 | 0.004 | 4 | 0.533 |
| 144 | hrs | Square Wave | 8.03 | 0.04 | 0.0032 | 3.2 | 0.399 |
| 1 | hr | Example 4 | 12.6 | 0.2 | 0.016 | 16 | 1.270 |
| 11 | hrs | Direct Current | 12.91 | 0.035 | 0.0028 | 2.8 | 0.217 |
| 24 | hrs | | 13.1 | 0.01 | 0.0008 | 0.8 | 0.061 |
| 58 | hrs | | 13.03 | 0.01 | 0.0008 | 0.8 | 0.061 |
| 1 | hr | Example 5 | 17.5 | 0.2 | 0.016 | 16 | 0.914 |
| 1 | hr | 10 Hz | 16.4 | 1.09 | 0.0872 | 87.2 | 5.317 |
| 36 | hrs | 0.1 sec/cycle | 13.1 | 0.46 | 0.0368 | 36.8 | 2.809 |
| 40 | hrs | Square Wave | 17.3 | 0.16 | 0.0128 | 12.8 | 0.740 |
| 46 | hrs | | 17.3 | 0.16 | 0.0128 | 12.8 | 0.740 |
| 1 | hr | Example 6 | 2 | 0.2 | 0.016 | 16 | 8.000 |
| 1 | hr | 10 Hz | 8 | 1.09 | 0.0872 | 87.2 | 10.900 |
| | | 0.1 sec/cycle | 14 | 0.46 | 0.0368 | 36.8 | 2.629 |
| 36 | hrs | Square Wave | 16.4 | 1.09 | 0.0872 | 87.2 | 5.317 |
| 40 | hrs | | 17.3 | 0.46 | 0.0368 | 36.8 | 2.127 |
| 46 | hrs | | 17.3 | 0.16 | 0.0128 | 12.8 | 0.740 |

### Amines as Bases

In addition, the invention and the inventive process can be carried out by treating the solution of the carboxylic acid or derivative thereof with a tertiary amine, secondary amine, primary amine or ammonia.

The invention can be carried out by replacing the alkali hydroxide with a tertiary amine, a secondary amine, a primary amine or ammonia salt in order to form the carboxylate salt. The amine base can be immobilized in a solid matrix and will allow for easy separation of products, such as using AMBERLYST A21 RESIN, a Divinyl Styrene copolymer with a tertiary amine functionality.

TABLE VII
Chemicals, pharmaceuticals, biopharmaceuticals, polymers and
surfaces that can be used as a substrate reactant. Using
reverse polarity electrolysis and the adducts that can be used
as Organic Radical Cations, Neutral Radicals, Cations, and
Anions included in Table I as Reactive Intermediates that may
be generated and undergo reactions under polarity Reversal
Electrolysis Conditions that undergo further reaction with the
substrate for new compounds and products.

| Substrate Compound | Additive Compound (Table I) |
|---|---|
| Fatty Acids | Hydrocarbons, Aromatics |
| Unsaturated and | Alcohols, Ethers, Aldehydes, Epoxides |
| Saturated | |
| Drugs | Ketones, Carboxylic Acids, Phenols, Epoxides |
| Pharmaceuticals | Nitrogen compounds, Epoxides |
| New Chemical Entities | Halogens, Allylic |
| Peptides | Small Radicals and Ions |
| Proteins | OH radical HOO radical HCO₃ radical |
| | CO₃⁻ radical |
| Biopharmaceuticals | Stable Neutral Molecules |
| Polymers | Hydrides: H₂O HCN HNCO |
| Surfaces | HOCN HNCS HSCN HF HCl |
| Medical Devices | Polyethylene Glycol, |
| Controlled Release | Water Soluble polymers and oligomers |
| Substrates | |
| Membrane surfaces | Hydrophobic and Hydrophilic groups and |
| | polymers |
| Chiral Compounds | Chiral intermediates, drugs, pharmaceuticals |

Any substrate compound under the substrate column can be reacted with any substance under the additive column to produce a new compound or modified compound or new product using the invention.

The invention has been described with reference to various specific and preferred embodiments and techniques. However, it should be understood that many variations and modifications may be made while remanding within the spirit and scope of the invention.

What is claimed is:

1. A polarity-reversal electrolysis process, comprising:
providing a reactor that comprises at least one pair of spaced electrodes;
providing a controlled polarity-reversing power supply that is constructed and arranged to provide polarity-reversed power to the electrodes;
providing to the reactor an electrically-conductive liquid reaction medium that comprises precursor reactants, wherein the electrodes are at least partially immersed in the reaction medium; and
operating the power supply such that the polarity of the electrodes of the pair of electrodes reverses at a frequency rate to produce reactive intermediates and products.

2. A product produced by the process of claim 1.

3. A product produced by the process of claim 1 wherein the products are selected from the group consisting of specialty chemicals, active pharmaceutical ingredients, pharmaceuticals, drugs, biologicals, antibiotics, insecticides and antifungals.

4. The process of claim 1 wherein the reactive intermediates formed at each electrode during the anodic cycle react with reactants selected from the group consisting of an alkyl group, an alkene group, an alkoxy group, an aryloxy group, an aryl group, a hydroxyl group, an epoxide group, and another nucleophile or electrophile capable of reacting with the intermediate.

5. The process of claim 1 wherein the reactive intermediates formed at each electrode during the cathodic cycle react with reactants selected from the group consisting of an alkyl group, an alkene group, an alkoxy group, an aryloxy group, an aryl group, a hydroxyl group, an epoxide group, and another nucleophile or electrophile capable of reacting with the intermediate.

6. The process of claim 1 wherein the reactive intermediates are generated from a chemical, active pharmaceutical ingredient, pharmaceutical, drug, biologic, antibiotic, insecticide or antifungal at each electrode during the anodic cycle and react with reactants selected from the group consisting of an alkyl group, an alkene group, an alkoxy group, an aryloxy group, ethylene glycol group, a water soluble polymer, a biocompatible polymer, polyethylene glycol group, an aryl group, a hydroxyl group, an epoxide group, and another nucleophile or electrophile capable of reacting with the reactive intermediate to produce a modified drug, pharmaceutical, pharmaceutical intermediate, biologic, antibiotic, insecticide or antifungal.

7. The process of claim 1 wherein the reactive intermediates are generated from a chemical, active pharmaceutical ingredient, pharmaceutical, drug, biologic, antibiotic, insecticide or antifungal at each electrode during the cathodic cycle and react with reactants selected from the group consisting of an alkyl group, an alkene group, an alkoxy group, an aryloxy group, ethylene glycol group, a water soluble polymer, a biocompatible polymer, polyethylene glycol group, an aryl group, a hydroxyl group, an epoxide group, and another nucleophile or electrophile capable of reacting with the reactive intermediate to produce a modified drug, pharmaceutical, pharmaceutical intermediate, biologic, antibiotic, insecticide or antifungal.

8. The process of claim 1 wherein the reactive intermediates are generated from a polymer containing an anionic group, at each electrode during the anodic cycle and react with reactants selected from the group consisting of an alkyl group, an alkene group, an alkoxy group, an aryloxy group, ethylene glycol group, a water soluble polymer, a biocompatible polymer, polyethylene glycol group, a polymer, a peptide, a protein, a drug, a pharmaceutical, an aryl group, a hydroxyl group, an epoxide group, and another nucleophile or electrophile capable of reacting with the reactive intermediate to produce a polymer-adduct or polymer graft.

9. The process of claim 1 wherein the reactive intermediates are generated from a polymer containing an anionic group, at each electrode during the cathodic cycle and react with reactants selected from the group consisting of an alkyl group, an alkene group, an alkoxy group, an aryloxy group, ethylene glycol group, a water soluble polymer, a biocompatible polymer, polyethylene glycol group, a polymer, a peptide, a protein, a drug, a pharmaceutical, an aryl group, a hydroxyl group, an epoxide group, and another nucleophile or electrophile capable of reacting with the reactive intermediate to produce a polymer-adduct or polymer graft.

10. The process of claim 1 wherein the reactive intermediates are generated from a polymer surface on a catheter or medical device, containing an anionic group, at each electrode during the anodic cycle and react with reactants selected from the group consisting of an alkyl group, an alkene group, an alkoxy group, an aryloxy group, ethylene glycol group, polyethylene glycol group, a water soluble polymer, a biocompatible polymer, a polymer, a peptide, a protein, a drug, a pharmaceutical, an aryl group, a hydroxyl group, an epoxide group, and another nucleophile or electrophile capable of reacting with the reactive intermediate to produce a polymer-adduct or polymer graft.

11. The process of claim 1 wherein the reactive intermediates are generated from a polymer surface on a catheter or medical device containing an anionic group, at each electrode during the cathodic cycle and react with reactants selected from the group consisting of an alkyl group, an alkene group, an alkoxy group, an aryloxy group, ethylene glycol group, polyethylene glycol group, a water soluble polymer, a biocompatible polymer, a polymer, a peptide, a protein, a drug, a pharmaceutical, an aryl group, a hydroxyl group, an epoxide group, and another nucleophile or electrophile capable of reacting with the reactive intermediate to produce a controlled release device for the reactant.

12. A product produced by the process of claim 1 wherein the product is selected from the group consisting of modified candidates for active pharmaceutical ingredients, pharmaceuticals, drugs, biologics, antibiotics, insecticides and antifungals.

13. A product produced by the process of claim 1 wherein the product is selected from the group consisting of modified active pharmaceutical ingredients, pharmaceuticals, drugs, biologics, antibiotics, insecticides and antifungals, each with increased water solubility as compared to the unmodified product.

14. A product produced by the process of claim 1 wherein the product is selected from the group consisting of modified active pharmaceutical ingredients, pharmaceuticals, drugs, biologics, antibiotics, insecticides and antifungals, each with decreased toxicity as compared to the unmodified product.

15. A product produced by the process of claim 1 wherein the product is selected from the group consisting of modified active pharmaceutical ingredients, pharmaceuticals, drugs, biologics, antibiotics, insecticides and antifungals, each with increased product half-life as compared to the unmodified product

16. A product produced by the process of claim 1 wherein the product is selected from the group consisting of modified active pharmaceutical ingredients, pharmaceuticals, drugs, biologics, antibiotics, insecticides and antifungals, each with decreased toxicity as compared to the unmodified product.

17. A product produced by the process of claim 1 wherein the products are able to be used to treat a particular medical indication or condition.

18. A product produced by the process of claim 1 wherein the products belong to at least one class of drugs that are used to treat a particular medical indication or condition.

19. A product produced by the process of claim 1 wherein the product is used as at least one of allergenics, anti-infectives, antifungals, antimalarial agents, antituberculosis agents, antiviral agents, carbapenems, cephalosporins, glycopeptide antibiotics, antineoplastics, biologicals, cardiovascular agents, antiarrhythmic agents, antihypertensive agents, diuretics, central nervous system agents, analgesics, anticonvulsants, antiparkinson agents, coagulation modifiers, gastrointestinal agents, genitourinary tract agents, hormones, immunologic agents, metabolic agents, plasma expanders, psychotherapeutic agents, radiological agents, radiopharmaceuticals, respiratory agents, and topical agents, to treat a particular medical indication.

20. A product produced by the process of claim 1 wherein the product is an azole antifungal derived from pyrazole, imidazole, thiazole, oxazole, or isoxazole.

21. A product produced by the process of claim 1 wherein the product is an angiotensin-converting enzyme inhibitor selected from the group consisting of modified Captopril, Zofenopril, Enalapril, Ramipril, Quinapril, Perindopril, Benazepril, Imidapril, Trandolapril, Cilazapril, Fosinopril, Peptides, and Lisinopril.

22. An apparatus for accomplishing polarity-reversal electrolysis, comprising:
a reactor that comprises at least one pair of spaced electrodes, wherein the electrodes are made from a material selected from the group consisting of carbon nanotubes, metal nanotubes, single walled nanotubes, double walled nanotubes, multiwalled nanotubes, carbon nanofibers, metal nanofibers, carbon nanoparticles, metal nanoparticles, graphene, graphene oxide, graphite, polymer, and combinations thereof;
a polarity-reversing power supply that is adapted to provide polarity-reversed power to the electrodes; and
a controller that controls at least the current, and the polarity reversal frequency, of the power supplied to the electrodes by the power supply.

23. The apparatus of claim 22 wherein the space between the said electrodes is from 1 nm to 100 micrometers.

24. The apparatus of claim 22 wherein the electrodes comprise nanotubes and the reactants flow through nanotubes embedded in an electrode matrix.

25. The apparatus of claim 22 wherein the electrodes comprise nanotubes and the reactants flow around the nanotubes embedded in an electrode matrix.

26. The apparatus of claim 22 wherein the nanotubes inside diameter is from 1 nm to 100 micrometers.

27. The apparatus of claim 22 wherein the nanotubes contain at least one of transition metal and transition metal oxide catalyst particles.

28. The apparatus of claim 22 wherein the nanotubes contain at least one of transition metals and metal oxides of platinum, nickel, Raney nickel, manganese, rhodium, palladium, iron, vanadium and titanium.
